(19) United States
(12) Patent Application Publication
(10) Pub. No.: US 2009/0304744 A1
(43) Pub. Date: Dec. 10, 2009

(54) METHODS OF ENHANCING IMMUNE RESPONSE IN THE INTRADERMAL COMPARTMENT AND COMPOUNDS USEFUL THEREOF

(75) Inventors: Robert L. Campbell, Bahama, NC (US); Kevin G. Dolan, Holly Springs, NC (US); Jason B. Alarcon, Durham, NC (US); John A. Mikszta, Durham, NC (US); Wendy Woodley, Cary, NC (US); Sheetal Mehta, East Windsor, NJ (US)

Correspondence Address: David W. Highet, VP & Chief IP Counsel;Becton, Dickinson and Company, (Alston & Bird LLP), 1 Becton Drive, MC 110, Franklin Lakes, NJ, 07417-1880, US

(73) Assignee: Becton, Dickinson and Company, Franklin Lakes, NJ (US)

(21) Appl. No.: 12/368,864

(22) Filed: Feb. 10, 2009

Related U.S. Application Data
(62) Division of application No. 11/006,369, filed on Dec. 6, 2004, now abandoned.
(60) Provisional application No. 60/527,599, filed on Dec. 5, 2003.

(51) Int. Cl. A61K 39/145; A61P 37/04

(52) U.S. Cl. 424/210.1; 424/209.1

(57) ABSTRACT

The present invention relates to immunogenic compositions for intradermal delivery of an antigenic or immunogenic agent in combination with one or more excipients. The immunogenic compositions of the invention comprise an antigenic or immunogenic agent and at least one excipient which acts as an adjuvant, i.e., enhances the immune response to the antigenic or immunogenic agent, once delivered to the intradermal compartment of a subject's skin. The immunogenic compositions of the invention comprise an excipient which when administered to the intradermal compartment of skin in accordance with the invention demonstrate adjuvant activity. The immunogenic compositions of the invention have enhanced efficacy as the excipients of the composition cause an asymptomatic skin irritation and recruit antigen presenting cells to the intradermal compartment and thus enhance presentation and/or availability of the antigenic or immunogenic agent to the antigen presenting cells. The enhanced efficacy of the immunogenic compositions of the invention may result in a therapeutically effective immune response after a single intradermal dose, with lower doses of antigenic or immunogenic agent than conventionally used, and without the need for booster immunizations.

### CROSS REFERENCE TO RELATED APPLICATIONS

This application is a divisional application of U.S. Utility application Ser. No. 11/006,369, filed Dec. 6, 2004, which claims the benefit of U.S. Provisional Application No. 60/527,599 filed Dec. 5, 2003, both of which are herein incorporated by reference in their entirety.

### 1. FIELD OF THE INVENTION

The present invention relates to immunogenic compositions for dermal delivery of an antigenic or immunogenic agent in combination with one or more excipients. The immunogenic compositions of the invention comprise an antigenic or immunogenic agent and at least one excipient which acts as an adjuvant, i.e., enhances the immune response to the antigenic or immunogenic agent, once delivered to the dermal compartment of a subject's skin, e.g., either the intradermal or the epidermal. The immunogenic compositions of the invention comprise an excipient which when administered to the intradermal compartment of skin in accordance with the invention demonstrate adjuvant activity. Alternatively, the immunogenic compositions of the invention comprise an excipient which when administered to the epidermal compartment of skin in accordance with the invention demonstrate adjuvant activity. The immunogenic compositions of the invention have enhanced efficacy as the excipients of the composition cause an asymptomatic skin irritation and recruit antigen presenting cells to the dermal compartment and thus enhance presentation and/or availability of the antigenic or immunogenic agent to the antigen presenting cells. The enhanced efficacy of the immunogenic compositions of the invention may result in a therapeutically and/or prophylactically effective immune response after a single dermal dose, with lower doses of antigenic or immunogenic agent than conventionally used, and without the need for booster immunizations.

### 2. BACKGROUND OF THE INVENTION

Pharmaceutical dosage forms contain both active ingredients, and inactive ingredients called excipients. The behavior of the dosage form is dependent on process variables and the interrelationship between the various excipients and their impact on the active ingredient. Excipients are therefore employed to effect various characteristics that improve the behavior of the dosage form to achieve better efficacy. For example, excipients are used in a pharmaceutical formulation to achieve higher stability, better resistance to biological or chemical deterioration, higher solubility and/or reduced surface tension for ease of delivery.

Adjuvants are agents that enhance the efficacy and protective immune response of an immunogenic formulation, e.g., vaccines. Traditionally, the immunogenicity of a vaccine formulation has been improved by incorporating an adjuvant in the formulation. Immunological adjuvants were initially described by Ramon (1924, Ann. Inst. Pasteur, 38: 1) as “substances used in combination with a specific antigen that produced a more robust immune response than the antigen alone.” Adjuvants differ from conventional excipients in that they directly enhance the efficacy of the active ingredient, i.e., immunogenicity, in an immunogenic formulation.

A wide variety of substances, both biological and synthetic, have been used as adjuvants. However, despite extensive evaluation of a large number of candidates over many years, the only adjuvants currently approved by the U.S. Food and Drug administration are aluminum-based minerals (generically called Alum). Alum has a debatable safety record (see, e.g., Malakoff, 2000, Science, 288: 1323), and comparative studies show that it is a weak adjuvant for antibody induction to protein subunits and a poor adjuvant for cell-mediated immunity. Moreover, Alum adjuvants can induce IgE antibody response and have been associated with allergic reactions in some subjects (see, e.g., Gupta et al., 1998, Drug Deliv. Rev. 32: 155-72; Relyveld et al., 1998, Vaccine 16: 1016-23). Many experimental adjuvants have advanced to clinical trials since the development of Alum, and some have demonstrated high potency but have proven too toxic for therapeutic use in humans.

Furthermore, the efficacy of adjuvants varies depending on the target compartment in a subject for the delivery of vaccines, and thus each adjuvant must be validated according to the vaccine's contemplated target compartment. Whereas hundreds of adjuvants or potential adjuvants have been found and validated for spaces other than the intradermal compartment, e.g., intramuscular, subcutaneous, prior to the instant invention there were only a limited number of traditional adjuvants showing promise in the intradermal compartment, and specifically no reported excipients with adjuvant activity in the intradermal compartment. Therefore, there is an unmet need for adjuvants that can effectively enhance immune response triggered by an intradermally administered immunogen.

### 3. SUMMARY OF THE INVENTION

The present invention is based, in part, on the inventors' unexpected discovery that intradermal delivery of an antigenic or immunogenic agent in combination with one or more pre-selected excipients results in an enhanced immune response to the antigenic or immunogenic agent. Preferably, excipients used in the methods and immunogenic compositions of the invention have not been previously associated with an adjuvant activity. Most preferably, excipients used in the methods and immunogenic compositions of the invention have not been previously associated with an adjuvant activity in the intradermal space. Although not intending to be bound by a particular mechanism of action, when the excipients of the instant invention are administered at the concentrations and by the delivery routes in accordance with the methods of the invention, they exhibit non-specific adjuvant activity, i.e., not through a specific cellular receptor, but perhaps through promotion of mechanical damage, mild irritation, or stretching of the skin. The enhanced efficacy of the intradermal vaccine formulations of the invention are based, in part, on the appreciation and recognition by the inventors that the intradermal compartment provides an ideal immunological space for a direct access of the antigenic or immunogenic agent to the immune cells residing therein. Indeed, the intradermal compartment has rarely been effectively targeted as a site of delivery of an antigenic or immunogenic agent, at least, in part, due to the difficulty of a specific and reproducible delivery of the antigenic or immunogenic agent, i.e., the precise needle placement into the intradermal space and adequate pressures of delivery.

The benefits of the invention are also appreciated in other dermal compartments including but not limited to the epidermal compartment of skin. Although not intending to be bound by any particular mechanism of action, the skin represents an attractive target site for delivery of vaccines and gene therapeutic agents. In the case of vaccines (both genetic and conventional), the skin is an attractive delivery site due to the high concentration of antigen presenting cells (APC) and APC precursors found within this tissue, especially the epidermal Langerhan's cells (LC) and the immune cells in the intradermal compartment.

The enhanced efficacy of the formulations of the inventions may be achieved with dermal vaccine formulations including formulations for intradermal and epidermal delivery. In some embodiments, the dermal vaccine formulations of the invention (including the epidermal and intradermal formulations) comprise an antigenic or immunogenic agent, and at least one excipient, which enhances the presentation and/or availability of the antigenic or immunogenic agent to an immune cell, e.g., the immune cells of the intradermal compartment (e.g., antigen presenting cells) or the immune cells of the epidermal compartment (e.g., epidermal Langerhan's cells (LC)), resulting in an enhanced immune response, preferably a protective immune response. In a specific embodiment, the molecule acts to prolong the exposure of the antigenic or immunogenic agent to the immune cells of the dermal compartment, e.g., antigen presenting cells, epidermal Langerhan's cells (LC), resulting in an enhanced protective immune response.

The invention encompasses immunogenic compositions for dermal delivery (including intradermal and epidermal delivery) comprising an antigenic or immunogenic agent, and at least one excipient, which enhances the immune response to the antigenic or immunogenic agent resulting in an enhanced immune response. In some embodiments, the excipients used in the immunogenic compositions of the invention allow the exposure of the antigenic or immunogenic agent to the immune cells of the intradermal compartment, by recruiting antigen presenting cells to the site of injection, resulting in an enhanced immune response to the antigenic or immunogenic agent.

The methods and compositions of the invention not only provide an enhanced immune response, enhanced therapeutic and/prophylactic efficacy in comparison to other conventional modes of delivery of immunogenic compositions (including intramuscular and subcutaneous delivery) but also provide reduced irritation at the injection site, enhanced mean titer antibody production as measured using methods known to the skilled artisan and exemplified herein; enhanced median antibody titers as measured using methods known to the skilled artisan and exemplified herein; enhanced rates of seroprotection and enhanced rates of seroconversion as measured using methods known to the skilled artisan and exemplified herein. The formulations of the invention reduce, preferably avoid hemolysis as measured using methods known to the skilled artisan and exemplified herein. The formulations of the invention also avoid gelling and other complication associated with altered viscosity that can hinder storage, preparation and administration.

Excipients that may be used in the immunogenic compositions of this invention include, but are not limited to, stabilizers, preservatives, solvents, surfactants or detergents, suspending agents, tonicity agents, vehicles and ingredients for growth medium. A non-limiting list of excipients that may be used in the immunogenic compositions of the invention are acetic acid, citric acid, fumaric acid, hydrochloric acid, nitric acid, sodium acetate, cellulose, charcoal, gelatin, ammonia solution, ammonium carbonate, mono-, di- or tri-ethanolamine, potassium hydroxide, sodium borate, sodium carbonate, sodium hydroxide, trolamine, nitrogen gas, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite, sodium sulfite, glycine, potassium metaphosphate, potassium phosphate, monobasic sodium acetate, anhydrous or dihydrate sodium citrate, edetate disodium, edetic acid, glycerin, propylene glycol and sorbitol, amphotericin B, benzoic acid, methyl-, ethyl-, propyl- or butyl-paraben, sodium benzoate and sodium propionate, amiprilose, benzalkonium chloride, benzethonium chloride, benzyl alcohol, betapropiolactone, cetylpyridium chloride, chlorobutanol, chlortetracycline, EDTA, formaldehyde, gentamicin, kanamycin, neomycin, phenol, phenoxyethanol, phenylethyl alcohol, phenylmercuric nitrate, polymyxin B, streptomycin, thimerosal, tri-(n)-butyl phosphate, nystatin, water, alcohol especially ethyl alcohol, corn oil, cottonseed oil, glycerin, isopropyl alcohol, mineral oil, oleic acid, peanut oil, purified water, water for injection, sterile water for injection, benzalkonium chloride, magnesium stearate, nonoxynol 10, oxtoxynol 9(TRITON® N-101), poloxamers such as poloxamer 124, 188(LUTROL® F-68), 237, 388, 403(P123) or 407 (LUTROL® F-127), polysorbate 20 (TWEEN™20), polysorbate 80 (TWEEN™80), sodium lauryl sulfate, sorbitan monopalmitate, agar, bentonite, carbomer (e.g., Carbopol), carboxymethylcellulose sodium, gelatin, hydroxyethyl cellulose, hydroxypropyl cellulose, hydroxypropyl methylcellulose, kaolin, methylcellulose, tragacanth, veegum, carboxymethylcellulose sodium, gelatin or methylcellulose, dextrose, glucose, sodium chloride, corn oil, mineral oil, peanut oil, sesame oil, bacteriostatic sodium chloride, bacteriostatic water, amino acids, bactopeptone, bovine albumin, bovine serum, egg protein, human serum albumin, mouse serum proteins, MRC-5 cellular protein, ovalbumin, vitamins, yeast proteins, apo-transferrin, aprotinin, anti-foaming agents such as polydimethylsilozone, silicon, fetuin (a serum protein), glycolic acid (a skin exfoliate), hydrogen peroxide (a detoxifier), lactose (a filler), mannose and urea.

The invention further encompasses other compounds or agents, which have not been previously associated with an adjuvant activity in any tissue space, that enhance the immune response triggered by the immunogenic or antigenic agent when co-administered intradermally with the immunogenic or antigenic agent. The invention particularly encompasses compounds or agents which have not been previously associated with an adjuvant activity in the intradermal compartment.

The concentration of the excipient used in the immunogenic compositions of the invention depends on the particular excipient used. In some embodiments, the concentration of the excipient used in the immunogenic compositions of the invention may be at 0.000002% to 58% (w/v) and 0.05% to 10% (v/v). In other embodiments, the concentration of the excipient used may be at least 10% (w/v), at least 15% (w/v), at least 20% (w/v), at least 25% (w/v), or at least 30% (w/v). In other embodiments, the concentration of the excipient is greater than about 30% (w/v). In yet other embodiments, the concentration of the excipient is at least 0.1% (w/v), at least 0.5% (w/v), at least 1% (w/v), at least 5% (w/v), or at least 10% (w/v). A number of the foregoing excipients may be used in the preparation and manufacturing of immunogenic compositions. In such cases, residual concentrations of the excipient may be found in the final immunogenic composition, left over from the manufacturing or preparation of the composition. However, such residual concentrations are too low to result in the adjuvant activity observed with the immunogenic compositions of the invention.

Antigenic or immunogenic agents that may be used in the immunogenic compositions of the invention include antigens from an animal, a plant, a bacteria, a protozoan, a parasite, a virus or a combination thereof. The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuraminidase, influenza virus hemagglutinin, herpes simplex viral protein, e.g., herpes simplex virus glycoprotein including for example, gB, gC, gD, and gE. The antigenic or immunogenic agent for use in the compositions of the invention may be an antigen of a pathogenic virus such as, an antigen of adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), metapneumovirus (e.g., avian pneumovirus and human metapneumovirus), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses), lentivirus (e.g. human immunodeficiency virus 1 and human immunodeficiency virus 2), spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus). The formulations of the invention may also improve prophylaxis against influenza, HIV, Polio, Dengue, Streppneumo, Pertusis, Herpes, HPV and Chlamydia diseases.

Alternatively, the antigenic or immunogenic agent in the immunogenic compositions of the invention may be a cancer or tumor antigen including but not limited to, KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens such as: CEA, TAG-72, CO17-1A; GICA 19-9, CTA-1 and LEA, Burkitt's lymphoma antigen-38.13, CD19, human B-lymphoma antigen-CD20, CD33, melanoma specific antigens such as ganglioside G_{D2}, ganglioside G_{D3}, ganglioside G_{M2}, ganglioside GM3, tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen, differentiation antigen such as human lung carcinoma antigen L6, L20, antigens of fibrosarcoma, human leukemia T cell antigen-Gp37, neoglycoprotein, sphingolipids, breast cancer antigen such as EGFR (Epidermal growth factor receptor), HER2 antigen (p185^{HER2}), polymorphic epithelial mucin (PEM), malignant human lymphocyte antigen-APO-1, differentiation antigen such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos, and T cell receptor derived peptide from a Cutaneous T cell Lymphoma.

The antigenic or immunogenic agent for use in the immunogenic compositions of the invention may be any substance that under appropriate conditions results in an immune response in a subject, including, but not limited to, polypeptides, peptides, proteins, glycoproteins, lipids, nucleic acids and polysaccharides. The concentration of the antigenic or immunogenic agent in the immunogenic compositions of the invention may be determined using standard methods known to one skilled in the art and depends on the potency and nature of the antigenic or immunogenic agent. Given the enhanced delivery system of the invention, the concentration of the antigenic or immunogenic agent is preferably less than the conventional amounts used when alternative routes of administration are employed and alternative compositions. The immunogenic agent for use in the compositions of the invention may also be a disrupted virion.

The immunogenic compositions of the invention are particularly advantageous for developing rapid and high levels of immunity against the antigenic or immunogenic agent, against which an immune response is desired. The immunogenic compositions of the invention can achieve a systemic immunity at a protective level with a low dose of the antigenic or immunogenic agent. In some embodiments, the immunogenic compositions of the invention result in an enhanced immune response with a dose of the antigenic or immunogenic agent which is 60%, preferably 50%, more preferably 40% of the dose conventionally used for the antigenic or immunogenic agent in obtaining an effective immune response, thus translating into a reduction in dose. In other embodiments, the immunogenic compositions of the invention result in an enhanced immune response with a dose of the antigenic or immunogenic agent which is at least 2-fold, at least 4-fold, at least 6-fold, at least 8-fold, at least 10-fold less than the dose conventionally used for the antigenic or immunogenic agent in obtaining an effective immune response.

In preferred embodiments, the immunogenic compositions of the invention comprise a dose of the antigenic or immunogenic agent which is lower than the conventional dose used in the art, e.g., the dose recommended in the Physician's Desk Reference, utilizing the conventional modes of delivery, e.g., intramuscular and subcutaneous and the conventional compositions, i.e., in the absence of excipients of the invention. Preferably, the immunogenic compositions of the invention result in a therapeutically or prophylactically effective immune response after a single intradermal dose. The immunogenic compositions of the invention may be administered intradermally for annual immunizations.

The immunogenic compositions of the instant invention have an enhanced therapeutic efficacy, safety, and toxicity profile relative to currently available formulations. The benefits and advantages imparted by the immunogenic compositions of the invention is, in part, due to the particular formulation and their utility in targeting the intradermal compartment of skin. Preferably, the immunogenic compositions of the invention may provide a greater and more durable protection, especially for high risk populations (e.g., elderly, infants, immunocompromised) that do not respond well to immunization.

The excipients for use in the methods and compositions of the invention having adjuvant properties in the intradermal space possess desirable immunopotentiation and tissue comparability attributes as determined using standard methods known in the art and disclosed herein. The preferred excipients of the invention have a common operating profile in the intradermal space defined by a slope (m) value of greater than 0.125. An exemplary profile for determining the optimal operating profile is depicted in FIG. 35. The slope identifies the change in maximum operating concentration as it relates to tissue depth within the intradermal compartment. Specifically, as illustrated in FIG. 35, the slope value is derived from a first and second excipient concentration and a first and second tissue depth within the intradermal compartment. The first reference point in the intradermal space is the more shallow delivery where the excipient demonstrates immunopotentiating properties with a draize score of 2 or less. The concentration of excipient at the first reference point is the highest concentration at the shallowest delivery depth that allows a draize score of 2 or less. The second reference point in the intradermal space is the deepest delivery where the excipient demonstrates immunopotentiating properties with a draize score of 2 or less. The concentration of excipient at the second reference point is the highest concentration at the deepest delivery depth that allows a draize score of 2 or less. For example, the distance between the first and second delivery depth can be 2 mm apart and specifically 1 mm and 3 mm deliveries. The operating slope (m) can be described by the following formula:

$$\frac{{C}_{2} - {C}_{1}}{{D}_{2} - {D}_{1}} = m$$

Where C₂ equals C_{infinity} at D₂

Where C₁ equals maximum excipient concentration at D₁ with a Draize score of 2 or less

Where Dₙ denotes delivery depth

While the slope criteria has many applications, it has particular utility when selecting excipients for vaccine delivery. For example, excipients are initially and preferentially screened at the shallow depths of 1.0-1.5 mm where ID delivery is readily confirmed by a bleb. Having a target slope value as a clear objective, reduces subsequent screening at the deeper tissue depths, reducing experimentation, time and costs. Most importantly, the slope value allows the formulation scientist to identify the excipients having the potential for greater immune enhancements.

The invention encompasses a composition for administration to the intradermal compartment of a subject's skin comprising an excipient, so that the composition demonstrates an adjuvant activity and a draize score that is equal to or less than two when delivered to the intradermal compartment.

The invention further encompasses composition for administration to the intradermal compartment of a subject's skin comprising an excipient, wherein the activity of the compositions can be characterized as a slope value equal to or greater than 0.125 when the composition is administered at a concentration that has both an adjuvant activity and a Draize score of less than or equal to 2, whereby the slope value is derived from a first and a second excipient concentration at a first and a second tissue depth within the intradermal compartment of the subject's skin, wherein the first and second tissue depths are at least 2 mm apart.

In some embodiments, the excipients of the invention have a narrow operating range, i.e., the range at which they have adjuvant activity in the intradermal compartment while having a draize score of equal to or less than two. In other embodiments, the excipients of the invention have a broad operating range, i.e., the range at which they have adjuvant activity in the intradermal compartment while having a draize score of equal to or less than two.

The invention encompasses a method for eliciting an enhanced immune response to an antigenic or immunogenic composition in a subject, preferably an animal, more preferably a human, comprising delivering an immunogenic composition into an intradermal compartment of the subject's skin, wherein the immunogenic composition comprises an antigenic or immunogenic agent and an excipient. In a specific embodiment, the immunogenic composition is a vaccine.

The invention further encompasses methods of identifying a compound that enhances an immune response to an immunogenic or antigenic agent. In one embodiment, a method of identifying a compound that enhances an immune response to an antigenic or immunogenic agent comprises: delivering an immunogenic composition into an intradermal compartment of a subject's skin, measuring a level of immune response, wherein the immunogenic composition comprises the immunogenic or antigenic agent and the compound and wherein the immune response is directed to the antigenic or immunogenic agent. The invention encompasses measuring a level of immune response by determining humoral and/or cell-mediated immune response using methods known to one skilled in the art and disclosed herein. Once a level of immune response is determined, it is compared to a standard level, wherein elevation of the measured level indicates that the compound is an adjuvant.

The invention further encompasses kits comprising an intradermal administration device and an immunogenic composition of the invention as described herein. In some embodiments, the invention provides a pharmaceutical pack or kit comprising an immunogenic composition of the invention. In a specific embodiment, the invention provides a kit comprising, one or more containers filled with one or more of the components of the immunogenic compositions of the invention, e.g., an antigenic or immunogenic agent, an excipient. In another specific embodiment, the kit comprises two containers, one containing an antigenic or immunogenic agent, and the other containing the excipient. Associated with such container(s) can be a notice in the form prescribed by a governmental agency regulating the manufacture, use or sale of pharmaceuticals or biological products, which notice reflects approval by the agency of manufacture, use or sale for human administration.

The invention further contemplates kits comprising an intradermal administration device and an intradermal vaccine formulation of the invention as described herein. The invention further contemplates kits comprising a dermal administration device and a dermal vaccine formulation of the invention as described herein. The invention further contemplates kits comprising an epidermal administration device and an epidermal vaccine formulation of the invention as described herein.

### 3.1 DEFINITIONS

As used herein, and unless otherwise specified, the term “excipient” means an ingredient or an additive in a pharmaceutical composition, which itself possesses no pharmacological or biological activity for which the composition is intended, and which prior to the instant invention not known to directly enhance or otherwise alter such pharmacological or biological activity when administered to the intradermal compartment of skin in accordance with the present invention. Excipients used in the methods of the present invention are pre-selected excipients. As used herein, “pre-selected” excipients encompass traditional, non-traditional, and any other exicipient that has an adjuvant activity when delivered to the intradermal compartment of a subject's skin in accordance with the methods of the invention.

As used herein, a “traditional” excipient, is a more or less inert substance added in a composition as a diluent or vehicle. Alternatively, a traditional excipient may be used to give form or consistency to a composition. Examples of such traditional excipients are known to one skilled in the art and encompassed within the instant invention, see, e.g., Remington's Pharmaceutical Sciences, Mack Pub. Co., N.J., current edition; all of which is incorporated herein by reference in its entirety.

As used herein a “traditional” adjuvant, is a substance added to a composition to enhance the antigenicity of the active ingredient in the composition, e.g., a suspension of minerals, on which an antigenic or immunogenic agent is absorbed, or water-in-oil emulsion in which an antigenic agent is emulsified in mineral oil (e.g., Freunds incomplete adjuvant) sometimes with the inclusion of killed mycobacteria to further enhance the antigenicity of the antigenic agent.

Seroconversion rate is defined as the percentage of recipients who have at least a 4-fold increase in serum haemagglutinin inhibition (HI) titers after vaccination, for each vaccine strain. Conversion factor is defined as the fold increase in serum HI geometric mean titers after vaccination, for each vaccine strain. Protection rate or seroprotection rate is defined as the percentage of recipients with a serum HI titer equal to or greater than 1:40 after vaccination and is normally accepted as indicating protection.

As used herein, the term “adjuvant” refers to any compound that assists or modifies the action of an agent, including but not limited to immunological adjuvants, which increase or diversify the immune response to an antigen. The term also encompasses compounds which when added to an immunogenic or antigen agent, non-specifically enhance an immune response to the agent in the recipient host upon exposure to the mixture. Adjuvants includes compounds that “immunomodulate” the cytokine network, up-regulating the immune response. Concomitant with this immunomodulation there is also a selection of which T-cell, Th1 or Th2, will mount this immune response. Th1 responses will elicit complement fixing antibodies and strong delayed-type hypersensitivity reactions associated with IL-2, and gamma-interferon. Induction of CTL response appears to be associated with a TH1 response. Th2 responses are associated with high levels of IgE, and the cytokines IL-4, IL-5, IL-6 and IL-10. The term adjuvants includes compounds which, when administered to an individual or tested in vitro, increase the immune response to an antigen in a subject to which the antigen is administered, or enhance certain activities of cells from the immune system. Some antigens are weakly immunogenic when administered alone or are toxic to a subject at concentrations that evoke useful immune responses in a subject. An adjuvant can enhance the immune response of the subject to the antigen by making the antigen more strongly immunogenic. The adjuvant effect can also result in the ability to administer a lower dose of antigen to achieve a useful immune response in a subject.

As used herein, the term “antigen” refers to a molecule which contains one or more epitopes capable of stimulating a host's immune system to make a cellular antigen-specific immune response when the antigen is presented in accordance with the present invention, or a humoral antibody response. An antigen may be capable of eliciting a cellular or humoral response by itself or when present in combination with another molecule. Normally, an epitope will include between about 3-15, preferably about 5-15, and more preferably about 7-15 amino acids. Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. The term “antigen” as used herein denotes both subunit antigens, i.e., antigens which are separate and discrete from a whole organism with which the antigen is associated in nature, as well as killed, attenuated, disrupted or inactivated bacteria, viruses, parasites or other microbes. Similarly, an oligonucleotide or polynucleotide which expresses a therapeutic or immunogenic protein, or antigenic determinant in vivo, such as in gene therapy and nucleic acid immunization applications, is also included in the definition of antigen herein. Further, for purposes of the present invention, antigens can be derived from any of several known viruses, bacteria, parasites and fungi, as well as any of the various tumor antigens. Furthermore, for purposes of the present invention, an “antigen” refers to a protein which includes modifications, such as deletions, additions and substitutions (generally conservative in nature), to the native sequence, so long as the protein maintains the ability to elicit an immunological response. These modifications may be deliberate, as through site-directed mutagenesis, or may be accidental, such as through mutations of hosts which produce the antigens.

As used herein, the term “immunological response” or “immune response” to an antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of endogenous cytokines, chemokines and other such molecules produced by activated T-cells and/or other white blood cells, including those derived from CD4+ and CD8+T-cells.

As used herein, and unless otherwise specified, the term “enhanced immune response” means that, when an antigenic or immunogenic agent of the invention is co-administered with one or more adjuvants of the invention, there is an increased antibody formation, measured using any standard methods known in the art and described in Section 5.4, below, in a subject that receives such an administration as compared to a subject to which same amount of the antigenic or immunogenic agent alone is administered. Preferably, an enhanced immune response means about 10%, 20%, 30%, 50%, 70%, or 100% or greater increase in antibody formation.

Alternatively, the term “enhanced immune response,” as used herein, means that, when an antigenic or immunogenic agent of the invention is co-administered with one or more adjuvant compounds of the invention, a smaller amount of the antigenic or immunogenic agent can be used to achieve the same level of antibody formation in a subject, as compared to a subject to which the antigenic or immunogenic agent alone is administered. Preferably, the antigenic or immunogenic compound in an amount of about 90%, 80%, 70%, 60%, 50%, 40%, 30% or less of the amount of the same agent administered without the adjuvant compounds of the invention, may be administered to achieve the same level of antibody formation in a subject when administered together with the adjuvant compound of the invention.

### 4. BRIEF DESCRIPTION OF FIGURES

FIG. 1. SERUM RESPONSE. Serum Response (1:123 Dil) to Flu Antigen Coat: Balb/c Mice Receiving Flu Vaccine vs. Adjuvanted-Flu Vaccine and Non-Immune (Tween 80 and Amiprilose Examples)

FIG. 2 SERUM RESPONSE. Serum Response (1:123 Dil) to Flu Antigen Coat: Balb/c Mice Receiving Flu Vaccine vs. Adjuvanted-Flu Vaccine and Non-Immune (Bactopeptone and Sodium Sulfite) (All ID delivered)

FIG. 3 SERUM RESPONSE. Serum Response (1:123 Dil) to Flu Antigen Coat: Balb/c Mice Receiving Flu Vaccine vs. Adjuvanted-Flu Vaccine and Non-Immune (Triton X-100) (All ID delivered)

FIG. 4A SERUM RESPONSE. Serum Response (1:123 Dil) to Flu Antigen Coat: Balb/c Mice Receiving Flu Vaccine vs. Adjuvanted-Flu Vaccine and Non-Immune (Sorbitol and Amphotericin B) (All ID delivered)

FIG. 4B Six point ELISA Assay showing sorbitol enhances Fluzone Trivalent Vaccine by 3×.

FIG. 5 SERUM RESPONSE. Serum Response (1:123 Dil) to Flu Antigen Coat: Balb/c Mice Receiving Flu Vaccine vs. Adjuvanted-Flu Vaccine and Non-Immune (Urea and Triton N-101) (All ID delivered)

FIG. 6 SERUM RESPONSE. Serum Response to Flu Antigen: Flu pDNA Immunogen vs. pDNA Supplemented with Fetuin (2^{nd} TB at 1:123 dilution)

FIG. 7 SERUM RESPONSE. Serum Response to Flu Antigen: Flu pDNA Immunogen vs. pDNA Supplemented with Methyl Cellulose, Gelatin, Bactopeptone and Tri-(N)-Butyl Phosphate (1^{st} TB at 1:370 dilution)

FIG. 8 SERUM RESPONSE. Serum Response to Flu Antigen: Flu pDNA Immunogen vs. pDNA Supplemented with Gelatin, Urea and Aprotinin (1^{st} TB at 1:123 dilution)

FIG. 9 SERUM RESPONSE. Serum Response to Flu Antigen: Flu pDNA Immunogen vs. pDNA Supplemented with ETOH and Sorbitol (1^{st} TB at 1:123 dilution)

FIG. 10 SERUM RESPONSE. Serum Response to Flu Antigen: Flu pDNA Immunogen vs. pDNA Supplemented with Sodium Sulfite (1^{st} TB at 1:370 dilution)

FIG. 11 SERUM RESPONSE. Serum Response to Flu Antigen: Flu pDNA Immunogen vs. pDNA Supplemented with Mannose, Apo-Transferrin, Glycolic Acid and Tween 20 (1^{st} TB at 1:370 dilution)

FIG. 12 NEEDLE DEVICE. An exploded, perspective illustration of a needle assembly designed according to this invention.

FIG. 13 NEEDLE DEVICE. A partial cross-sectional illustration of the embodiment in FIG. 12.

FIG. 14 NEEDLE DEVICE. Embodiment of FIG. 13 attached to a syringe body to form an injection device.

FIGS. 15A-B MICROABRADER DEVICE.

A. an elevated view of the handle end of a preferred embodiment

B. a side view of a preferred embodiment of a microabrader.

FIGS. 16A-B MICROABRADER DEVICE.

A. is a transparent perspective view of the microabrader device of FIGS. 15A and 15B.

B. is a cross sectional view of the microabrader device of FIG. 15B.

FIG. 17 MICROABRADER DEVICE is a side view of the abrading surface the microabrader device of FIGS. 15A, 15B, 16A, and 16B on the skin of a subject.

FIG. 18 MICROABRADER DEVICE

A. is a perspective view of the abrading surface in the embodiment of FIG. 17.

B. is a cross sectional side view of the abrader surface.

FIG. 19 MICROABRADER DEVICE: a bottom view of the abrader surface of the embodiment of FIG. 17.

FIG. 20 MICROABRADER DEVICE: a perspective view in partial cross section of abraded furrows of skin.

FIG. 21 TWEEN 80 ADJUVANT PROPERTIES IN THE ID SPACE. Tween 80 at 5% led to 100% seroconversion in one study.

FIG. 22 DRAIZE SCORING AT INJECTION SITES. In swine skin irritation studies 5% Tween 80 was well tolerated in the ID space.

FIG. 23 COMPARISON OF ID VS. IM DELIVERY FOR INFLUENZA VACCINE (MURINE MODEL). Tween 80 (0.9% V/V) delivered ID with a trivalent vaccine led to higher mean titers, higher median titers and higher seroconversion as compared to the commercial trivalent vaccine delivered IM.

FIG. 24 COMPARISON OF TWEEN 80 AND SORBITOL. Tween 80 was not well tolerated at 10% V/V. In contrast the 10% W/V sorbitol was well tolerated.

FIG. 25 SKIN COMPATIBILITY PROFILES AS A FUNCTION OF NEEDLE DEPTH: Swine data at 20-24 hours post administration showed how a 2% TWEEN™ 80 solution was tolerated when delivered with 1.0 mm, 1.5 mm, 2.0 mm and 3.0 mm needle. Skin reactions improved with depth. Deeper tissue is more tolerant. Higher concentrations of Tween 80 with greater adjuvant strength can be used with deeper tissue.

FIG. 26 IMMUNOGENICITY OF FLUZONE SUPPLEMENTED WITH GELATIN: IM DELIVERY V. ID DELIVERY: A Fluzone® trivalent formula supplemented with gelatin was delivered ID and straight Fluzone® was delivered IM. 0.45% w/v gelatin enhanced seroconversion and median titer.

FIG. 27 SKIN COMPATIBILITY STUDIES: Swine tolerated up to 600 ng/100 ul or 1200 ng/200 ul total amphotericin per dose as evident by the Draize score analysis. Draize score determined 1 hour post administration.

FIG. 28 IMMUNE RESPONSE OF FLUZONE SUPPLEMENTED WITH DEOXYCHOLATE: ID V. IM DELIVERY (ID+/−Deoxycholate): When deoxycholate, was delivered to the ID space it had immunopotentiating characteristics. In IM delivery, only 1 in 5 animals seroconverted 21 days after immunization. In ID delivery, however 5 of 5 animals seroconverted. ID delivery resulted in the best median titer.

FIG. 29 SKIN COMPATIBILITY PROFILES AS A FUNCTION OF NEEDLE DEPTH. Concentrations of deoxycholate at 0.5% W/V and higher could not be tolerated at the 1.5 mm depth. Draize score determined 1 hour post administration.

FIG. 30 SKIN COMPATIBILITY PROFILES OF BACTOPEPTONE. Skin presentation immediately after the last injection. The excipient, bactopeptone, has a calming affect.

FIG. 31 FLUZONE® IIMUNOGENICY ENHANCED IN GUINEA PIG MODEL WITH 5.0% V/V TWEEN™ 80. Comparison of IM and ID delivery of Fluzone® in the presence or absence of TWEEN™ 80. In an HAI assay with trivalent antigen (New Calcdonia, Panama, B-Hong Kong), ID delivery of Fluzone Fluzone® supplemented with TWEEN™ 80 outperformed Fluzone Fluzone® Delivered ID without supplement and Fluzone® delivered IM without supplement.

FIG. 32 FLUZONE® IIMUNOGENICITY ENHANCED IN GUINEA PIG MODEL WITH 0.1% V/V SODIUM DEOXYCHOLATE. Comparison of IM and ID delivery of Fluzone® in the presence or ansence of Deoxycholate. In an HAI assay with trivalent antigen (New Calcdonia, Panama, B-Hong Kong), ID delivery of Fluzone® supplemented with sodium deoxycholate outperformed Fluzone® Delivered ID without supplement and Fluzone® delivered IM without supplement.

FIG. 33 DRAIZE SCORING OF VARIOUS EXCIPIENTS IN HARTLEY GUINEA PIGS. Excipients tested, at specified concentration, were well tolerated in guinea pigs.

FIG. 34 DRAIZE SCORING OF VARIOUS EXCIPIENTS IN YORKSHIRE SWINE. Excipients tested, at specified concentration, were well tolerated in swine.

FIG. 35 IDEAL EXCIPIENT PROPERTIES Excipient A has the desired profile. The maximum concentration tolerated at the 1 mm depth can be substantially increased by administering to deeper intradermal tissue and thereby having the potential to gain further immunologic benefits. An excipient with a slope (maximum acceptable conc./tissue depth) greater than or equal to 0.125 is preferred.

### 5. DETAILED DESCRIPTION OF THE INVENTION

The invention encompasses immunogenic compositions for intradermal delivery comprising an antigenic or immunogenic agent, and at least one excipient, which enhances the immune response to the antigenic or immunogenic agent resulting in an enhanced immune response. In some embodiments, the immunogenic compositions result in an enhanced immune response. Although not intending to be bound by a particular mechanism of action, when the excipients of the instant invention are administered at the concentrations and by the delivery routes in accordance with the methods of the invention, they exhibit non-specific adjuvant activity, i.e., not through a specific cellular receptor, but perhaps through promotion of mechanical damage, mild irritation, or stretching of the skin. Alternatively, although not intending to be bound by a particular mechanism of action, once the excipients are delivered to the intradermal compartment of a subject's skin, they may act as a skin irritant leading to the recruitment of antigen presenting cells to the intradermal compartment at the site of the injection, and thus act as an adjuvant, i.e., enhance the immune response to the immunogenic composition. Preferably, excipients used in the methods and immunogenic compositions of the invention have not been previously associated with an adjuvant activity. Most preferably, excipients used in the methods and immunogenic compositions of the invention have not been previously associated with an adjuvant activity in the intradermal space.

The methods and compositions of the invention not only provide an enhanced immune response, enhanced therapeutic and/prophylactic efficacy in comparison to other conventional modes of delivery of immunogenic compositions (including intramuscular and subcutaneous delivery) but also provide reduced irritation at the injection site, enhanced mean titer antibody production as measured using methods known to the skilled artisan and exemplified herein; enhanced median antibody titers as measured using methods known to the skilled artisan and exemplified herein; enhanced rates of seroconversion and seroprotection as measured using methods known to the skilled artisan and exemplified herein; reduced hemolysis as measured using methods known to the skilled artisan and exemplified herein, reduced geling during storage and preparation.

Excipients that may be used in the immunogenic compositions of this invention include, but are not limited to, stabilizers, preservatives, solvents, surfactants or detergents, suspending agents, tonicity agents, vehicles and ingredients for growth medium. A non-limiting list of excipients that may be used in the immunogenic compositions of the invention are acetic acid, citric acid, fumaric acid, hydrochloric acid, nitric acid, sodium acetate, cellulose, charcoal, gelatin, ammonia solution, ammonium carbonate, mono-, di- or tri-ethanolamine, potassium hydroxide, sodium borate, sodium carbonate, sodium hydroxide and trolamine, nitrogen gas, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite and sodium sulfite, glycine, potassium metaphosphate, potassium phosphate, monobasic sodium acetate, anhydrous or dihydrate sodium citrate, edetate disodium, edetic acid, glycerin, propylene glycol, sorbitol, amphotericin B, benzoic acid, methyl-, ethyl-, propyl- or butyl-paraben, sodium benzoate and sodium propionate, amiprilose, benzalkonium chloride, benzethonium chloride, benzyl alcohol, betapropiolactone, cetylpyridium chloride, chlorobutanol, chlortetracycline, EDTA, formaldehyde, gentamicin, kanamycin, neomycin, phenol, phenoxyethanol, phenylethyl alcohol, phenylmercuric nitrate, polymyxin B, streptomycin, thimerosal, tri-(n)-butyl phosphate, nystatin, water, alcohol especially ethyl alcohol, corn oil, cottonseed oil, glycerin, isopropyl alcohol, mineral oil, oleic acid, peanut oil, purified water, water for injection, sterile water for injection, benzalkonium chloride, chloride, magnesium stearate, nonoxynol 10, oxtoxynol 9 (TRITON® N-101), poloxamers such as poloxamer 124, 188 (Lutrol® F-68), 237, 388 or 407 (Lutrol® F-127), polysorbate 20 (TWEEN™20), polysorbate 80 (TWEEN™80), sodium lauryl sulfate, sorbitan monopalmitate, agar, bentonite, carbomer (e.g., Carbopol), carboxymethylcellulose sodium, gelatin, hydroxyethyl cellulose, hydroxypropyl cellulose, hydroxypropyl methylcellulose, kaolin, methylcellulose, tragacanth and veegum, carboxymethylcellulose sodium, gelatin or methylcellulose, dextrose, glucose, sodium chloride, corn oil, mineral oil, peanut oil, sesame oil, bacteriostatic sodium chloride, bacteriostatic water, amino acids, bactopeptone, bovine albumin, bovine serum, egg protein, human serum albumin, mouse serum proteins, MRC-5 cellular protein, ovalbumin, vitamins, yeast proteins, apo-transferrin, aprotinin, anti-foaming agents such as polydimethylsilozone, silicon, fetuin (a serum protein), glycolic acid (a skin exfoliate), hydrogen peroxide (a detoxifier), lactose (a filler), mannose and urea.

The concentration of the excipient used in the immunogenic compositions of the invention depends on the particular excipient used. In some embodiments, the concentration of the excipient used in the immunogenic compositions of the invention may be at 0.000002% to 58% (w/v) and 0.05% to 10.0% (v/v). In other embodiments, the concentration of the excipient used may be at least 10% (w/v), at least 15% (w/v), at least 20% (w/v), at least 25% (w/v), or at least 30% (w/v). In other embodiments, the concentration of the excipient is greater than about 30% (w/v). In yet other embodiments, the concentration of the excipient is at least 0.1% (w/v), at least 0.5% (w/v), at least 1% (w/v), at least 5% (w/v), or at least 10% (w/v). Excipients may be used in the preparation and manufacturing of immunogenic compositions. In such cases, residual concentrations of the excipient may be found in the final immunogenic composition, left over from the manufacturing or preparation of the composition. Such residual concentrations are too low to result in the adjuvant activity observed with the immunogenic compositions of the invention.

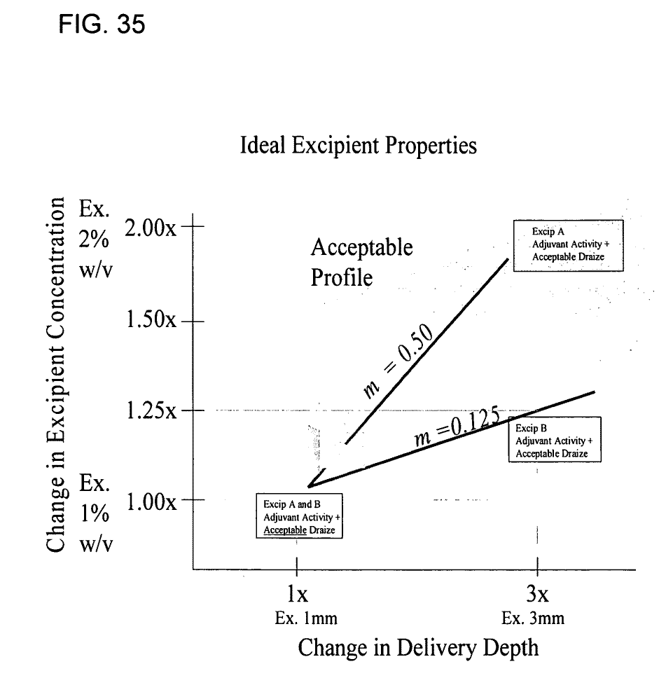

The excipients for use in the methods and compositions of the invention having adjuvant properties in the intradermal space possess desirable immunopotentiation and tissue compatability attributes as determined using standard methods known in the art and disclosed herein. The preferred excipients of the invention have a common operating profile in the intradermal space defined by a slope (m) value of greater than 0.125. An exemplary profile for determining the optimal operating profile is depicted in FIG. 35. The slope identifies the change in maximum operating concentration as it relates to tissue depth within the intradermal compartment. Specifically, as illustrated in FIG. 35, the slope value is derived from a first and second excipient concentration and a first and second tissue depth within the intradermal compartment. The first reference point in the intradermal space is the more shallow delivery where the excipient demonstrates immunopotentiating properties with a draize score of 2 or less. The concentration of excipient at the first reference point is the highest concentration at the shallowest delivery depth that allows a draize score of 2 or less. The second reference point in the intradermal space is the deepest delivery where the excipient demonstrates immunopotentiating properties with a draize score of 2 or less. The concentration of excipient at the second reference point is the highest concentration at the deepest delivery depth that allows a draize score of 2 or less. For example, the distance between the first and second delivery depth can be 2 mm apart and specifically 1 mm and 3 mm deliveries. The operating slope (m) can be described by the following formula:

$$\frac{{C}_{2} - {C}_{1}}{{D}_{2} - {D}_{1}} = m$$

Where C₂ equals C_{infinity} at D₂

Where C₁ equals maximum excipient concentration at D₁ with a Draize score of 2 or less

Where Dₙ denotes delivery depth

The invention encompasses a composition for administration to the intradermal compartment of a subject's skin comprising an excipient, so that the composition demonstrates an adjuvant activity and a draize score that is equal to or less than two when delivered to the intradermal compartment.

The invention further encompasses composition for administration to the intradermal compartment of a subject's skin comprising an excipient, wherein the activity of the compositions can be characterized as a slope value equal to or greater than 0.125 when the composition is administered at a concentration that has both an adjuvant activity and a Draize score of less than or equal to 2, whereby the slope value is derived from a first and a second excipient concentration at a first and a second tissue depth within the intradermal compartment of the subject's skin, wherein the first and second tissue depths are at least 2 mm apart.

In some embodiments, the excipients of the invention have a narrow operating range, i.e., the range at which they have adjuvant activity in the intradermal compartment while having a draize score of equal to or less than two. In other embodiments, the excipients of the invention have a broad operating range, i.e., the range at which they have adjuvant activity in the intradermal compartment while having a draize score of equal to or less than two.

Antigenic or immunogenic agents that may be used in the immunogenic compositions of the invention include antigens from an animal, a plant, a bacteria, a protozoan, a parasite, a virus or a combination thereof. The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuraminidase, influenza virus hemagglutinin, herpes simplex viral protein, e.g., herpes simplex virus glycoprotein including for example, gB, gC, gD, and gE. The antigenic or immunogenic agent for use in the compositions of the invention may be an antigen of a pathogenic virus such as, an antigen of adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), metapneumovirus (e.g., avian pneumovirus and human metapneumovirus), picornaviridae (e.g., enterovirus, rhinovirus, hepatovirus (e.g., human hepatitis A virus), cardiovirus, and apthovirus, reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses), lentivirus (e.g. human immunodeficiency virus 1 and human immunodeficiency virus 2), spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Alternatively, the antigenic or immongenic agent in the immunogenic compositions of the invention may be a cancer or tumor antigen including but not limited to, KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens such as: CEA, TAG-72, C017-1A; GICA 19-9, CTA-1 and LEA, Burkitt's lymphoma antigen-38.13, CD19, human B-lymphoma antigen-CD20, CD33, melanoma specific antigens such as ganglioside GD2, ganglioside GD3, ganglioside GM2, ganglioside GM3, tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen, differentiation antigen such as human lung carcinoma antigen L6, L20, antigens of fibrosarcoma, human leukemia T cell antigen-Gp37, neoglycoprotein, sphingolipids, breast cancer antigen such as EGFR (Epidermal growth factor receptor), HER2 antigen (p185^{HER2}), polymorphic epithelial mucin (PEM), malignant human lymphocyte antigen-APO-1, differentiation antigen such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos, and a T cell receptor derived peptide from a Cutaneous T cell Lymphoma.

The antigenic or immunogenic agent for use in the immunogenic compositions of the invention may be any substance that under appropriate conditions results in an immune response in a subject, including, but not limited to, polypeptides, peptides, proteins, glycoproteins, lipids, nucleic acids and polysaccharides. The concentration of the antigenic or immunogenic agent in the immunogenic compositions of the invention may be determined using standard methods known to one skilled in the art and depends on the potency and nature of the antigenic or immunogenic agent. Given the enhanced delivery system of the invention, the concentration of the antigenic or immunogenic agent is preferably less than the conventional amounts used when alternative routes of administration are employed and alternative compositions.

The invention further encompasses other compounds or agents, which have not been previously associated with an adjuvant activity in any tissue space, that enhance the immune response triggered by the immunogenic or antigenic agent when co-administered intradermally with the immunogenic or antigenic agent. The invention particularly encompasses compounds or agents which have not been previously associated with an adjuvant activity in the intradermal compartment.

The invention encompasses methods for intradermal delivery of the immunogenic compositions of the invention described and exemplified herein to the intradermal compartment of a subject's skin, preferably by directly and selectively targeting the intradermal compartment. The immunogenic compositions of the invention are administered using any of the intradermal devices and methods disclosed in U.S. patent application Ser. Nos. 09/417,671, filed on Oct. 14, 1999; 09/606,909, filed on Jun. 29, 2000; 09/893,746, filed on Jun. 29, 2001; 10/028,989, filed on Dec. 28, 2001; 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety.

The actual method by which the immunogenic composition of the invention are targeted to the intradermal space is not critical as long as it penetrates the skin of a subject to the desired targeted depth within the intradermal space without passing through it. The actual optimal penetration depth will vary depending on the thickness of the subject's skin. In most cases, skin is penetrated to a depth of about 0.5-2 mm. Regardless of the specific intradermal device and method of delivery, the intradermal delivery preferably targets the immunogenic composition of this invention to a depth of at least 0.3 mm, more preferably at least 0.5 mm up to a depth of no more than 2.0 mm, more preferably no more than 1.7 mm. In certain cases, the immunogenic compositions are delivered at a targeted depth just under the stratum corneum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhans cells in the dermal space of human skin, delivery would need to encompass, at least, in part, the epidermal tissue depth typically ranging from about 0.025 mm to about 0.2 mm in humans.

The invention provides methods of treatment and prophylaxis which involve administering an immunogenic composition of the invention to a subject, preferably a mammal, and most preferably a human for treating, managing or ameliorating symptoms associated with a disease or disorder, especially an infectious disease or cancer. The subject is preferably a mammal such as a non-primate, e.g., cow, pig, horse, cat, dog, rat, mouse and a primate, e.g., a monkey such as a Cynomolgous monkey and a human. In a preferred embodiment, the subject is a human. Preferably, the immunogenic composition of the invention is a vaccine composition.

The invention encompasses a method for immunization and/or stimulating an immune response in a subject comprising intradermal delivery of a single dose of a composition of the invention to a subject, preferably a human. In some embodiments, the invention encompasses one or more booster immunizations. The immunogenic composition of the invention is particularly effective in stimulating and/or up-regulating an antibody response to a level greater than that seen in conventional immunogenic compositions (such as vaccines) and administration schedules. The immunogenic compositions of the invention are particularly advantageous for developing rapid and high levels of immunity against the antigenic or immunogenic agent, against which an immune response is desired. The immunogenic compositions of the invention can achieve a systemic immunity at a protective level with a low dose of the antigenic or immunogenic agent. In some embodiments, the immunogenic compositions of the invention result in an enhanced immune response with a dose of the antigenic or immunogenic agent which is 60%, preferably 50%, more preferably 40% of the dose conventionally used for the antigenic or immunogenic agent in obtaining an effective immune response. In preferred embodiments, the immunogenic compositions of the invention comprise a dose of the antigenic or immunogenic agent which is lower than the conventional dose used in the art, e.g., the dose recommended in the Physician's Desk Reference, utilizing the conventional modes of delivery, e.g., intramuscular and subcutaneous and the conventional compositions, i.e., in the absence of excipients of the invention. Preferably, the immunogenic compositions of the invention result in a therapeutically or prophylactically effective immune response after a single intradermal dose. The immunogenic compositions of the invention may be administered intradermally for annual immunizations.

The immunogenic compositions of the instant invention have an enhanced therapeutic efficacy, safety, and toxicity profile relative to currently available formulations. The benefits and advantages imparted by the immunogenic compositions of the invention is, in part, due to the particular formulation and their utility in targeting the intradermal compartment of skin. Preferably, the immunogenic compositions of the invention provide a greater and more durable protection, especially for high risk populations that do not respond well to immunization.

The invention encompasses methods for determining the efficacy of immunogenic compositions of the invention using any standard method known in the art or described herein. Assays for determining the efficacy of the immunogenic compositions of the invention may be in vitro based assays or in vivo based assays, including animal based assays. In some embodiments, the invention encompasses detecting and/or quantitating a humoral immune response against the antigenic or immunogenic agent of a composition of the invention in a sample, e.g., serum, obtained from a subject who has been administered an immunogenic composition of the invention. Preferably, the humoral immune response of the immunogenic compositions of the invention are compared to a control sample obtained from the same subject, who has been administered a control formulation, e.g., a formulation which simply comprises of the antigenic or immunogenic agent.

In other embodiments, the invention encompasses methods for determining the efficacy of the compositions of the invention by measuring cell-mediate immune response. Methods for measuring cell-mediated immune response are known to one skilled in the art and encompassed within the invention. In some embodiments, a T cell immune response may be measured for quantitating the immune response in a subject, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

The invention further encompasses methods of identifying a compound that enhances an immune response to an immunogenic or antigenic agent. In one embodiment, a method of identifying a compound that enhances an immune response to an antigenic or immunogenic agent comprises: delivering an immunogenic composition into an intradermal compartment of a subject's skin, measuring a level of immune response, wherein the immunogenic composition comprises the immunogenic or antigenic agent and the compound and wherein the immune response is directed to the antigenic or immunogenic agent. The invention encompasses measuring a level of immune response by determining humoral and/or cell-mediated immune response using methods known to one skilled in the art and disclosed herein. Once a level of immune response is determined, it is compared to a standard level, wherein elevation of the measured level indicates that the compound is an adjuvant.

In a specific embodiment, a method for identifying a compound that enhances immunogenicity of an immunogenic or antigenic agent comprises: (a) delivering an immunogenic composition into an intradermal compartment of a first subject's skin, wherein the immunogenic composition comprises the immunogenic or antigenic agent and the compound; (b) measuring antibody response in a sample obtained from the first subject's serum; (c) delivering and immunogenic composition into an intradermal compartment of a second subject's skin, wherein the immunogenic composition comprises the immunogenic or antigenic agent without the compound, and wherein the first and the second subjects are same species; (d) measuring antibody response in a sample obtained from the second subject's serum; (e) determining whether the response obtained from the first subject is greater than the response obtained from the second subject. If the response in the sample obtained from the first subject is greater than the second subject, characterizing the compound as an excipient that may be used in the compositions of the invention, (f) demonstrating candidate formulation will pass through microneedle, and (g) demonstrating that the concentration of the agent that provides an adjuvant property is a concentration that produces acceptable draize scores. Compounds identified by the screening methods of the invention can be used to elicit an enhanced immune response to an antigenic or immunogenic agent when co-administered with the antigenic or immunogenic agent into an intradermal compartment of the subject's skin. Specifically, these compounds can be used in vaccine compositions.

The invention further encompasses kits comprising an intradermal administration device and an immunogenic composition of the invention as described herein. In some embodiments, the invention provides a pharmaceutical pack or kit comprising an immunogenic composition of the invention. In a specific embodiment, the invention provides a kit comprising, one or more containers filled with one or more of the components of the immunogenic compositions of the invention, e.g., an antigenic or immunogenic agent, an excipient. In another specific embodiment, the kit comprises two containers, one containing an antigenic or immunogenic agent, and the other containing the excipient. Associated with such container(s) can be a notice in the form prescribed by a governmental agency regulating the manufacture, use or sale of pharmaceuticals or biological products, which notice reflects approval by the agency of manufacture, use or sale for human administration.

5.1 Immunogenic Compositions

The immunogenic compositions of the invention are designed for targeted delivery of the antigenic or immunogenic agent, preferably, selectively and specifically, to the intradermal compartment of a subject's skin. In some embodiments, the immunogenic compositions of the invention are targeted directly to the intradermal compartment of skin. The immunogenic compositions of the invention comprise an antigenic or immunogenic agent and at least one excipient, which enhances the presentation and/or availability of the antigenic or immunogenic to an immune cell, such as the immune cells of the intradermal compartment, resulting in an enhanced immune response. The immunogenic compositions of the invention may enhance cell-mediated and/or humoral mediated immune response. Cell-mediated immune responses that may be modulated by the intradermal vaccine formulations of the invention include for example, Th1 or Th2 CD4+T-helper cell-mediated or CD8+ cytotoxic T-lymphocytes mediates responses.

Excipients that may be used in the immunogenic compositions of this invention include, but are not limited to, stabilizers, preservatives, solvents, surfactants or detergents, suspending agents, tonicity agents, vehicles and ingredients for growth medium. Examples of excipients that may be used in the compositions and methods of the invention are disclosed herein in Section 5.1.1 and exemplified in Examples 6.1-6.3. The concentration of the excipient used in the immunogenic compositions of the invention depends on the particular excipient used (See Section 5.1.1 and Examples 6.1-6.3. In some embodiments, the concentration of the excipient used in the immunogenic compositions of the invention may be at 0.000002% to 58% (w/v) and 0.05% to 10.0% (v/v). In other embodiments, the concentration of the excipient used maybe at least 10% (w/v), at least 15% (w/v), at least 20% (w/v), at least 25% (w/v), or at least 30% (w/v). In other embodiments, the concentration of the excipient is greater than about 30% (w/v). In yet other embodiments, the concentration of the excipient is at least 0.1% (w/v), at least 0.5% (w/v), at least 1% (w/v), at least 5% (w/v), or at least 10% (w/v). Excipients may be used in the preparation and manufacturing of immunogenic compositions. In such cases, residual concentrations of the excipient may be found in the final immunogenic composition, left over from the manufacturing or preparation of the composition. Such residual concentrations are too low to result in the adjuvant activity observed with the immunogenic compositions of the invention.

In some embodiments, the immunogenic compositions of the invention comprise one or more additives including, but not limited to, a traditional adjuvant, a traditional excipient, a stabilizer, a penetration enhancer, and a muco or bioadhesive. A traditional excipient, is a more or less inert substance added in a composition as a diluent or vehicle. Alternatively, a traditional excipient may be used to give form or consistency to a composition. Examples of such traditional excipients are known to one skilled in the art and encompassed within the instant invention, see, e.g., Remington's Pharmaceutical Sciences Mack Pub. Co., N.J., current edition; all of which is incorporated herein by reference in its entirety. A traditional adjuvant, is a substance added to a composition to enhance the antigenicity of the active ingredient in the composition, e.g., a suspension of minerals, on which an antigenic or immunogenic agent is absorbed, or water-in-oil emulsion in which an antigenic agent is emulsified in mineral oil (e.g., Freunds incomplete adjuvant) sometimes with the inclusion of killed mycobacteria to further enhance the antigenicity of the antigenic agent.

In other embodiments, the immunogenic compositions of the present invention may further comprise one or more other pharmaceutically acceptable carriers, including any suitable diluent or excipient. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the immunogenic compositions of the invention include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety).

In particular embodiments, the immunogenic compositions of the invention, may also contain wetting agents, emulsifying agents, or pH buffering agents. The immunogenic compositions of the invention can be a solid, such as a lyophilized powder suitable for reconstitution, a liquid solution, a suspension, a tablet, a pill, a capsule, a sustained release formulation, or a powder.

The immunogenic compositions of the invention may be in any form suitable for intradermal delivery. Preferably, the immunogenic compositions of the invention are stable formulations, i.e., undergo minimal to no detectable level of degradation and/or aggregation of the antigenic or immunogenic agent, and can be stored for an extended period of time with no loss in biological activity, e.g., antigenicity or immunogenicity of the antigenic agent.

5.1.1 Excipients

The invention is based, in part, on the unexpected discovery by the inventors that intradermal delivery of an antigenic or immunogenic agent in combination with one or more excipients results in an enhanced immune response to the antigenic or immunogenic agent. As used herein, and unless otherwise specified, the term “excipient” means an ingredient or an additive in a pharmaceutical composition, which itself possesses no pharmacological or biological activity for which the composition is intended, and which prior to the instant invention not known to directly enhance or otherwise alter such pharmacological or biological activity when administered to the intradermal compartment of skin in accordance with the present invention. Excipients used in the methods of the present invention are pre-selected excipients. As used herein, “pre-selected” excipients encompass traditional, non-traditional, and any other exicipient that has an adjuvant activity when delivered to the intradermal compartment of a subject's skin in accordance with the methods of the invention. It has been unexpectedly discovered that these excipients, when co-administered with an antigenic or immunogenic agent to the intradermal compartment act as an adjuvant, i.e., enhance the immune response to the antigenic or immunogenic agent in a subject receiving such composition as compared to a subject receiving the composition without the excipient. Preferably, the excipients used in the immunogenic compositions and methods of the invention have not been previously associated with an adjuvant activity. Most preferably, the excipients used in the immunogenic compositions and methods of the invention have not been previously associated with an adjuvant activity in the intradermal compartment.

The immunogenic compositions of the invention results in among other advantages, in a higher mean serum antibody response, higher antibody titers, higher rates of seroconversion and seroprotection relative to traditional modes of delivery, including IM. Measurement of such parameters is within the level of skill in the art and such methods are exemplified herein.

Although not intending to be bound by a particular mechanism of action, when the excipients of the instant invention are administered at the concentrations and by the delivery routes in accordance with the methods of the invention, they exhibit non-specific adjuvant activity, i.e., not through a specific cellular receptor, but perhaps through promotion of mechanical damage, mild irritation, or stretching of the skin. Alternatively, although not intending to be bound by a particular mechanism of action, once the excipients are delivered at the concentrations and to the intradermal compartment of a subject's skin in accordance with the present invention, they may act as a skin irritant leading to the recruitment of antigen presenting cells to the intradermal compartment at the site of the injection, and thus act as an adjuvant, i.e., enhance the immune response to the immunogenic composition.

As used herein, when an excipient acts as an irritant it causes a reversible an asymptomatic inflammatory effect on skin tissue by chemical action at the site of contact and yet is not corrosive. Inflammatory effect at the site of injection involves an influx of blood at the site of injection and may be marked by swelling, redness, heat, and/or pain. One skilled in the art can determine if an excipient is a skin irritant using, for example, the methods disclosed in Code of Federal Regulation (Title 16, Vol. 2; 6 CFR 1500.41, which is incorporated herein by reference in its entirety). According to 6 CFR 1500.41, a chemical is a skin irritant if, when tested on the intact skin of albino rabbits by the methods of 16 CFR 1500.41 for four hours exposure or by other appropriate techniques, it results in an empirical score of five or more. Preferably, the excipients used in the methods of the invention have a score of 5 or less, more preferably a score of 4 or less, and most preferably a score of 3 or less. When an excipient of the invention is characterized as a skin irritant, one or more other excipients that are not skin irritants may be used in the immunogenic compositions to reduce the skin irritation. In a specific embodiment, in order to determine if the immunogenic composition of the invention results in skin irritation, once the immunogenic composition, e.g., a vaccine, is delivered to the intradermal compartment of a subject's skin, e.g., an animal, the site of the injection is visually checked within one hour of the immunization, at 24 hours and again at 21 days. Any observation other than the initial “Bleb” which resolves in hours, would be noted. In a specific embodiment, when a DNA immunogenic agent, e.g., pDNA-HA is delivered to the intradermal compartment of a subject's skin, the site of the injection is checked within one hour of the immunization (prime or boost), 24 hours afterwards, at 21 days just before boost, 24 hours after the boost and 21 days after the boost (actual day 42 of schedule).

Excipients are typically classified into subclasses according to their function. Excipients used in the immunogenic compositions of the invention may have one or more function. Several subclasses of excipients are known in the art and are encompassed in the present invention. See, e.g., Ansel et al., Pharmaceutical Dosage Forms and Drug Delivery System, 6^{th} Ed., pp. 110-133, Williams & Wilkins (1995), which is incorporated herein by reference in its entirety. For example, an excipient can be categorized as a stabilizer, a preservative, a solvent, a surfactant or detergent, a suspending agent, a tonicity agent or a vehicle. In the case of vaccines, ingredients for growth medium, which are used to facilitate or maintain the growth of the immunogen, are commonly used as excipients. Some excipients have more than one function and can be used for multiple purposes. It will be apparent to those of ordinary skill in the art that these subclasses are not an exhaustive list of all available excipients, thus other types of excipients can also be used in accordance with the immunogenic compositions and methods of the invention. Additional categories and examples of excipients are provided in Handbook of Pharmaceutical Excipients, 2003 (4^{th} ed., American Pharmaceutical Association, London), the entirety of which is incorporated herein by reference.

In some embodiments, the excipients used in the immunogenic compositions of the invention are stabilizers. As used herein, a stabilizer is a chemical agent that increases the stability of a pharmaceutical composition. As used herein, a stable composition refers to a composition that undergoes minimal to no detectable level of degradation and/or aggregation of the antigenic or immunogenic agent, and can be stored for an extended period of time with no loss in biological activity, e.g., antigenicity or immunogenicity of the antigenic agent. Preferably, the immunogenic compositions of the present invention exhibit stability at the temperature ranges of 2° C.-8° C., preferably at 4° C., for at least 2 years, as assessed by high performance size exclusion chromatography (HPSEC). Preferably, the immunogenic compositions of the present invention to have low to undetectable levels of aggregation and/or degradation of the antigenic or immunogenic agent, after the storage for the defined periods as set forth above. Preferably, no more than 5%, no more than 4%, no more than 3%, no more than 2%, no more than 1%, and most preferably no more than 0.5%, of the antigenic or immunogenic molecule forms an aggregate or degrades as measured by HPSEC, after the storage for the defined periods as set forth above. In most preferred embodiments, the immunogenic compositions of the present invention will exhibit almost no loss in biological activity of the antigenic or immunogenic agent during a prolonged storage under the conditions described above, as assessed by standard methods known in the art. The immunogenic compositions of the present invention retain after the storage for the above-defined periods more than 80%, more than 85%, more than 90%, more than 95%, more than 98%, more than 99%, or more than 99.5% of the initial biological activity prior to the storage.

Depending on the mechanism by which an excipient stabilizes the composition, the stabilizers can be further categorized into an acidifying or alkalinizing agent, an adsorbent, an air displacement agent, an antioxidant, a buffering agent, a chelating agent or a humectant, which are all encompassed within the instant invention. An acidifying agent as used herein stabilizes a pharmaceutical composition by providing an acidic medium for the active ingredient in the composition, i.e., the antigenic or immunogenic agent, that is otherwise labile in an alkaline condition. Examples of an acidifying agent include, but are not limited to, acetic acid, citric acid, fumaric acid, hydrochloric acid, nitric acid and sodium acetate. An alkalinizing agent stabilizes the composition by providing an alkaline medium for the active ingredient in the composition, i.e., the antigenic or immunogenic agent that are labile in an acidic environment. Examples of an alkalinizing agent include, but are not limited to, ammonia solution, ammonium carbonate, mono-, di- or tri-ethanolamine, potassium hydroxide, sodium borate, sodium carbonate, sodium hydroxide and trolamine.

In a specific embodiment, the excipient used in the immunogenic composition of the invention is an adsorbent. An adsorbent as used herein is an agent capable of allowing other molecules to adhere or adsorb onto its surface by physical and/or chemical means. Examples of an adsorbent include, but are not limited to, cellulose, charcoal and gelatin. In a more specific embodiment, the excipient of this invention is gelatin. Preferably, gelatin is administered at a concentration of from about 0.01 to about 2 percent weight per volume of the composition, and more preferably, from about 0.03 to about 0.6 percent weight per volume of the composition. In another specific embodiment, gelatin is administered at a concentration of from about 0.0 to 0.225% weight per volume.

In some embodiments, the invention encompasses an excipient which is an antioxidant. Although not intending to be bound by a particular mechanism of action an antioxidant stabilizes a pharmaceutical composition by inhibiting oxidation, and thus preventing the deterioration of the composition by the oxidative process. Examples of an antioxidant for use in the immunogenic compositions of the invention include, but are not limited to, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite and sodium sulfite.

In a specific embodiment, the excipient used in the immunogenic compositions of the invention is an antioxidant. In a more specific embodiment, the excipient used in the immunogenic compositions of the invention is sodium bisulfite. Preferably, sodium bisulfite is used at a concentration of from about 0.1 to about 8.0 percent weight per volume of the composition, and more preferably, from about 0.3 to about 3.0 percent weight per volume of the composition.

The invention further encompasses excipients which are buffering agents. Although not intending to be bound by a particular mechanism of action a buffering agent stabilizes a pharmaceutical composition by providing resistance to alterations in pH for example, upon dilution or addition of acid or alkali. Examples of buffering agents that may be used in the immunogenic compositions of the invention include, but are not limited to, glycine, potassium metaphosphate, potassium phosphate, monobasic sodium acetate, and anhydrous or dihydrate sodium citrate.

The invention further contemplates chelating agents for use in the immunogenic compositions of the invention. Although not intending to be bound by a particular mechanism of action, a chelating agent stabilizes a pharmaceutical composition by forming a stable, water soluble complex with one or more metals, e.g., heavy metals. Heavy metals are typically critical in enzymatic activity of proteases, and thus chelating agents limit the activity of the proteases by sequestering a metal needed for their enzymatic activity. Examples of a chelating agents that may be used in the compositions of the invention include, but are not limited to, edetate disodium and edetic acid.

In some embodiments, the excipient used in the immunogenic compositions of the invention is a humectant. A humectant is an agent that prevents the drying out of preparations by retaining moisture. Examples of humectants that may be used in the immunogenic compositions of the invention include, but are not limited to, glycerin, propylene glycol and sorbitol. In a specific embodiment, the excipient of this invention is a humectant. In a more specific embodiment, the excipient of this invention is sorbitol. Preferably, sorbitol is administered at a concentration of from about 1 to about 100 percent weight per volume of the composition, and more preferably, from about 2.5 to about 70 percent weight per volume of the composition, and more preferably, from about 5 to about 20 percent weight per volume of the composition.

The invention further encompasses excipients which are preservatives. Although not intending to be bound by a particular mechanism of action a preservative is a substance that prevents the growth of exogenous organisms in a pharmaceutical composition. Preservatives include for example, antifungal agents, i.e., an agent that prevents the growth of fungi, and antimicrobial agents, i.e., an agent that prevents the growth of microorganisms including viruses. Examples of antifungal agents that may be used in the immunogenic compositions and methods of the invention include, but are not limited to, amphotericin B, benzoic acid, methyl-, ethyl-, propyl- or butyl-paraben, sodium benzoate and sodium propionate. In case of the parabens, it is well known that the effectiveness is usually enhanced when they are used in combination. Examples of antimicrobial agents that may be used in the immunogenic compositions and methods of the invention include, but are not limited to, amiprilose, benzalkonium chloride, benzethonium chloride, benzyl alcohol, betapropiolactone, cetylpyridium chloride, chlorobutanol, chlortetracycline, EDTA, formaldehyde, gentamicin, kanamycin, neomycin, phenol, phenoxyethanol, phenylethyl alcohol, phenylmercuric nitrate, polymyxin B, streptomycin, thimerosal, tri-(n)-butyl phosphate.

In a specific embodiment, the excipient used in the immunogenic compositions of the invention is an antifungal agent. In a more specific embodiment, the excipient used in the immunogenic compositions of the invention is amphotericin B. Preferably, amphotericin B is used at a concentration of from about 0.5 to about 600 ng/mL, and more preferably, from about 30 to about 100 ng/mL. In yet other embodiments, amphotericin B is used at a concentration of from about 0.1 ng/200 μL to 1200 ng/200 μL. Excipients used in the immunogenic compositions of the invention may be an amphoteric or polyenic antibiotic. Examples of amphoteric antibiotics that may be used in the immunogenic compositions of the invention include but are not limited to amphotericin B and Nystatin.

In another specific embodiment, the excipient used in the compositions of the invention is an antimicrobial agent. In a more specific embodiment, the excipient used in the immunogenic compositions of this invention is amiprilose or tri-(n)-butyl phosphate. Preferably, amiprilose is used at a concentration of from 0.1 to about 0.9% w/v. Preferably, tri-(n)-butyl phosphate is used at a concentration of from 0.04 to about 0.325% w/v.

The invention encompasses excipients which are solvents, i.e., an agent used to dissolve another pharmaceutical substance, in the preparation of a composition of the invention. The solvent may be used to dissolve the antigenic or immunogenic agent. The solvents used in the immunogenic compositions of the invention may be aqueous or non-aqueous. In some embodiments cosolvents are used in the compositions of the invention, e.g., water and alcohol. For preparation of an injectable compositions, it is preferable to use a sterilized solvent. Examples of solvents that may be used in the immunogenic compositions of the invention include, but are not limited to, alcohol, especially ethyl alcohol, corn oil, cottonseed oil, glycerin, isopropyl alcohol, mineral oil, oleic acid, peanut oil, purified water, water for injection, and sterile water for injection.

In a specific embodiment, the excipient used in the immunogenic compositions of the invention is a solvent. In a more specific embodiment, the excipient used in the immunogenic compositions of the invention is ethanol. In other specific embodiments, ethanol is used at a concentration of from about 0.01 to about 2.0 percent volume per volume of the composition, and preferably, from about 0.05 to about 0.45 percent volume per volume of the composition. In some specific embodiments, the concentration of the ethanol may be 2.0% v/v at the deeper intradermal depths, e.g., at a depth of greater than 1 mm.

The invention further encompasses surfactants, i.e., surface active agents, as excipients for use in the immunogenic compositions of the invention. Although not intending to be bound by a particular mechanism of action a surfactant absorbs to a surface or an interface and reduces surface or interfacial tension. A surfactant may be used as a wetting agent, detergent or emulsifying agent.

Examples of a surfactants that may be used in the compositions of the invention include, but are not limited to, benzalkonium chloride, magnesium stearate, nonoxynol 10, oxtoxynol 9 (TRITON® N-101), poloxamers such as poloxamer 124, 188 (LUTROL® F 68), 237, 388 or 407 (LUTROL® F 127), polysorbate 20 (TWEEN™ 20), polysorbate 80 (TWEEN™ 80), sodium lauryl sulfate, sorbitan monopalmitate and TRITON® X-100.

In a specific embodiment, the excipient used in the immunogenic compositions of the invention is a surfactant. In a more specific embodiment, the excipient of this invention is LUTROL® F 127, TRITON® N-101, TRITON® X-100, TWEEN™ 20 or TWEEN™ 80.

The invention encompasses non-ionic surfactant excipients which function as adjuvants when delivered to the ID compartment in accordance with the methods of the invention. Although not intending to be bound by a particular mechanism of action, the concentration range of such detergents that results in adjuvant properties in the intradermal compartment is narrow in contrast to the broad ranges reported in the literature where such detergents have been used for general vaccine manufacturing purposes. The preferred operating concentrations vary with needle depth (1.00 mm vs. 1.5 mm vs. 2.0 mm vs. 3.00 mm). The invention encompasses use of the non-ionic surfactant excipients at ranges where adjuvant properties are demonstrated while tissue irritation is avoided or minimized, with no toxicicity, or damage to the tissue. In most preferred embodiments, when such excipients are delivered to the ID compartment, there is an enhanced immune response as measured for example by an enhanced seroconversion, enhanced mean antibody titer or an enhanced median antibody titre (using methods known to the skilled artisan and exemplified herein). Non-ionic surfactants are often provided commercially as concentrated liquid stocks. Sigma-Aldrich Company products; Cat. T-6878, Cat. 303135, Cat. P-8074, Cat. P7949 or products of similar concentration and purity are useful in practicing this invention.

In a specific preferred embodiment, TRITON® N-101 is used at a concentration of from about 0.05 to about 5 percent weight per volume of the composition, and more preferably, from about 0.1 to about 1.5 percent weight per volume of the composition. The invention encompasses use of TRITON® N-101 at a concentration as high as 5% at deeper intradermal depths, e.g., at a depth greater than 2.5 mm.

In a specific preferred embodiment, TRITON® X-100 is used at a concentration of from about 0.00003 to about 5 percent weight per volume of the composition, and more preferably, from about 0.0001 to about 0.0009 percent weight per volume of the composition. The invention encompasses use of TRITON® X-100 at a concentration as high as 5% at deeper intradermal depths, e.g., at a depth greater than 2.5 mm.

In a specific preferred embodiment, TWEEN™ 80 is used at a concentration of from about 0.03 to about 3 percent weight per volume (w/v) of the composition, or from about 0.03 to about 5% w/v, 0.01 to about 10% w/v, and more preferably, from about 0.1 to about 0.9 percent weight per volume of the composition. In another preferred specific embodiment, the TWEEN™ 80 is used at a concentration of from about 1.1-2.0% v/v when the formulation is delivered to a depth of 2 mm or less in the intradermal compartment of skin. In yet another preferred specific embodiment, the TWEEN™ 80 is used at a concentration of from about 1.1-5.0% v/v when the formulation is delivered to a depth of 2 mm or greater in the intradermal compartment of skin. More specifically, TWEEN™ is used at 1.1 to 2.5% V/V at the 1.0-1.5 mm depth, 1.1 to 5.0% V/V at the 1.6 to 2 mm depth, 1.1 to 7.5% V/V at the 2.1 to 2.5 mm depth, and 1.1 to 10.0% V/V at the 2.6 to 3 mm depth.

In a specific preferred embodiment, TWEEN™ 20 is used at a concentration of from about 0.003 to 0.03% w/v and from about 0.003 to 0.3% w/v and 0.003 to 3.0% w/v. Expressed as V/V, in a preferred embodiment, TWEEN™ 20 is used at a concentration of from about 0.003 to 0.03% v/v and from about 0.003 to 0.3% v/v and 0.003 to 3.0% v/v.

In another specific embodiment, Sorbitol is used at a concentration of from about 2.0 to 10% w/v when the formulation is delivered to a depth of 2 mm or less in the intradermal compartment of skin. In yet another preferred specific embodiment, Sorbitol is used at a concentration of from about 2 to 20% w/v when the formulation is delivered to a depth of 2 mm or greater in the intradermal compartment of skin. Surfactants are typically used in the preparation and manufacturing of immunogenic compositions, particularly vaccines. In such cases, residual concentrations of the surfactant may be found in the final immunogenic composition, left over from the preparation or manufacturing of the composition. Such residual concentrations are too low to result in the adjuvant activity observed with the immunogenic compositions of the invention. Examples of such surfactants are octyl- or nonylphenoxy polyoxyethanols (e.g., TRITON® series), polyoxyethylene sorbitan esters (e.g., TWEEN™ series), and polyoxyethylene esters or ethers; Octylphenoxy polyoxyethanols and polyoxyethylene sorbitan esters including t-octylphenoxypolyoxyehtnaol; and Polyoxyethylene sorbitan esters including poloxyethylene sorbitan monooleate; TRITON® X-45, TRITON® X-102, TRITON® X-114, TRITON® X-165, TRITON® X-205, TRITON® X-305, TRITON® N-57, TRITON® N-101, TRITON® N-128, Breij 35, Laureth-9, Steareth-9, TWEEN™ 80.™. (For a list of surfactants see, e.g., Surfactant Systems, eds., Attwood and Florence, 1983, Chapman and Hall, which is incorporated herein by reference in its entirety).

The invention encompasses excipients for use in the immunogenic compositions of the invention which are suspending agents. Although not intending to be bound by a particular mechanism of action, a suspending agent increases the viscosity of the composition by for example reducing the rate of sedimentation of particles dispersed throughout a vehicle in which they are not soluble. Examples of suspending agents that may be used in the compositions of the invention include, but are not limited to, agar, bentonite, carbomer (e.g., Carbopol), carboxymethylcellulose sodium, gelatin, hydroxyethyl cellulose, hydroxypropyl cellulose, hydroxypropyl methylcellulose, kaolin, methylcellulose, tragacanth and veegum.

In one embodiment, the excipient of this invention is a suspending agent. In a more specific embodiment, the excipient of this invention is gelatin or methylcellulose. Preferably, methylcellulose is used at a concentration of from about 0.02 to about 0.5 percent weight per volume of the composition, and more preferably, from about 0.06 to about 0.18 percent weight per volume of the composition.

The invention encompasses a tonicity agent as an excipient for use in the compositions of the invention. Tonicity agents are particularly desired in the immunogenic compositions of the invention as they provide a solution with osmotic characteristics similar to physiologic fluid, and are thus optimal for injectable compositions of the invention. Examples of a tonicity agent that may be used in the immunogenic compositions of the invention include, but are not limited to, dextrose, glucose and sodium chloride.

The invention further encompasses an excipient which is a vehicle. As used herein vehicle is a carrying agent for a substance in a pharmaceutical composition. Vehicles are frequently used in formulating a variety of compositions for oral and parenteral administration. Vehicles for use in the methods and immunogenic compositions of the invention may be aqueous or oleaginous vehicles. Examples of a vehicle which may be used in the immunogenic compositions of the invention include, but are not limited to, corn oil, mineral oil, peanut oil, sesame oil, bacteriostatic sodium chloride injection and bacteriostatic water.

Growth medium ingredients may be used as excipients in the immunogenic compositions of the invention. Growth medium ingredients are particularly useful when the composition is a vaccine. Examples of growth medium ingredients that may be used in the immunogenic compositions and methods of the invention include, but are not limited to, amino acids, bactopeptone, bovine albumin, bovine serum, egg protein, human serum albumin, mouse serum proteins, MRC-5 cellular protein, ovalbumin, vitamins and yeast proteins.

In a specific embodiment, the excipient used in the immunogenic composition of the invention is a growth medium ingredient. In a more specific embodiment, the excipient in the immunogenic composition of the invention is bactopeptone. Preferably, bactopeptone is used at a concentration of from about 0.03 to about 3 percent weight per volume of the composition, and more preferably, from about 0.1 to about 1.5 percent weight per volume of the composition.

The invention encompasses other compounds or agents that have not been known to possess an adjuvant activity, particularly in the intradermal compartment. Examples of these compounds include, but are not limited to, serum protein (e.g., apo-transferrin, fetuin), aprotinin, glycolic acid (a skin exfoliate), mannose and urea. Any supplemental protein may possess an adjuvant activity when used in accordance with the methods of the present invention and delivered to the intradermal compartment of skin. Supplemental proteins are particularly useful as adjuvants for DNA immunogens. Compounds related to urea such as uric acid are anticipated to work according to the instant invention.

In a specific embodiment, the excipient used in the immunogenic compositions of the invention is apo-transferrin, aprotinin, fetuin, glycolic acid, mannose or urea. Preferably, urea is used at a concentration of from about 0.02 to about 40 percent weight per volume of the composition, and more preferably, from about 0.2 to about 20 percent weight per volume of the composition. Preferably, apo-transferrin is used at a concentration of from about 20 μg/mL to about 1,800 μg/mL of the composition, and more preferably, from about 60 μg/mL to about 600 μg/mL of the composition. Preferably, aprotinin is used at a concentration of from about 1 μg/mL to about 180 μg/mL of the composition, and more preferably, from about 5 μg/mL to about 60 μg/mL of the composition. Preferably, fetuin is used at a concentration of from about 0.05 μg/mL to about 7.5 μg/mL of the composition, and more preferably, from about 0.2 μg/mL to about 2.4 μg/mL of the composition. Preferably, mannose is used at a concentration of from about 20 μg/mL to about 1,800 μg/mL of the composition, and more preferably, from about 60 μg/mL to about 600 μg/ml of the composition. Preferably, glycolic acid is used at a concentration of from about 0.05 to about 3.0 percent weight per volume of the composition, and more preferably, from about 0.1 to about 1.0 percent weight per volume of the composition.

In yet another specific embodiment, the excipient used in the immunogenic compositions of the invention is a bile acid or a derivative thereof, including but not limited to deoxycholate (DOC), cholic acid, chendeoxycholic acid, lithocholic acid, hyodeoxycholic acid and ursodeoxycholic acid. In another specific embodiment, deoxycholate is used at a concentration of from about 0.07 to 0.15% w/v, or 0.01 to 0.3% w/v when the formulation is delivered to a depth of 2 mm or less in the intradermal compartment of skin. In yet another preferred specific embodiment, deoxycholate is used at a concentration of from about 0.07 to 0.15% w/v, or 0.01 to 0.6% w/v when the formulation is delivered to a depth of 2 mm or greater in the intradermal compartment of skin. More specifically the preferred range for DOC at 1 mm to 1.5 mm in depth is 0.07 to 0.15% w/v and the preferred range for DOC at 1.6 mm to 2.mm depth is 0.07 to 0.3% w/v and the preferred range for DOC at 2.1 mm to 2.5 mm depth is 0.07 to 0.45% w/v and the preferred range for DOC at 2.6 mm to 3.0 mm depth is 0.07 to 0.6% w/v.

The invention encompasses formulations comprising any excipient that matches the desired operating profile, as defined herein and exemplified in FIG. 35, having a slope greater than or equal to 0.125.

The excipients used in the immunogenic compositions of the invention can exist in a liquid or solid form. Further, it will be readily apparent to those of ordinary skill in the art that these excipients can be used alone or in combination with other excipients. Particularly, two or more excipients can be used in combination to achieve an additive or a synergistic effect. The concentration of the excipient in the immunogenic compositions of the invention does not include the residual concentration of the excipient that may be present from the preparation or manufacturing of the composition prior to preparation of the immunogenic composition in accordance with the methods of the instant invention.

5.1.2 Immunogenic or Antigenic Agents

Antigenic or immunogenic agents that may be used in the immunogenic composition of this invention include antigens from an animal, a plant, a bacteria, a protozoan, a parasite, a virus or a combination thereof. The antigenic or immunogenic agent for use in the immunogenic composition of this invention may be any substance that under appropriate conditions results in an immune response in a subject, including, but not limited to, polypeptides, peptides, proteins, glycoproteins, lipids, nucleic acids and polysaccharides.

The immunogenic composition of this invention may comprise one or more antigenic or immunogenic agents. The amount of the antigenic or immunogenic agent used in the compositions of this invention may vary depending on the chemical nature and the potency of the antigenic or immunogenic agent. Typically, the starting concentration of the antigenic or immunogenic agent in the composition of this invention is the amount that is conventionally used for eliciting the desired immune response, using the conventional routes of administration, e.g., intramuscular injection. The concentration of the antigenic or immunogenic agent in the composition of this invention is then adjusted, e.g., by dilution using a diluent, so that an effective protective immune response is achieved as assessed using standard methods known in the art and described herein.

The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuraminidase, influenza virus hemagglutinin, herpes simplex viral protein, e.g., herpes simplex virus glycoprotein including for example, gB, gC, gD, and gE.

The antigenic or immunogenic agent for use in the immunogenic composition of this invention may be an antigen of a pathogenic virus, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), and metapneumovirus (e.g., avian pneumovirus and human metapneumovirus), picornaviridae (e.g., enterovirus, rhinovirus, hepatovirus (e.g., human hepatitis A virus), cardiovirus, and apthovirus, reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus, e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

The antigenic or immunogenic agent used in the immunogenic composition of this invention may be an infectious disease agent including, but not limited to, influenza virus hemagglutinin (Genbank Accession No. JO2132; Air, 1981, Proc. Natl. Acad. Sci. USA 78: 7639-7643; Newton et al., 1983, Virology 128: 495-501), human respiratory syncytial virus G glycoprotein (Genbank Accession No. Z33429; Garcia et al., 1994, J. Virol.; Collins et al., 1984, Proc. Natl. Acad. Sci. USA 81: 7683), core protein, matrix protein or any other protein of Dengue virus (Genbank Accession No. M19197; Hahn et al., 1988, Virology 162: 167-180), measles virus hemagglutinin (Genbank Accession No. M81899; Rota et al., 1992, Virology 188: 135-142), herpes simplex virus type 2 glycoprotein gB (Genbank Accession No. M14923; Bzik et al., 1986, Virology 155:322-333), poliovirus I VP1 (Emini et al., 1983, Nature 304:699), envelope glycoproteins of HIV I (Putney et al., 1986, Science 234: 1392-1395), hepatitis B surface antigen (Itoh et al., 1986, Nature 308: 19; Neurath et al., 1986, Vaccine 4: 34), diptheria toxin (Audibert et al., 1981, Nature 289: 543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog cholera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales-Scarano et al., 1982, Virology 120: 42), neonatal calf diarrhea virus (Matsuno and Inouye, 1983, Infection and Immunity 39: 155), Venezuelan equine encephalomyelitis virus (Mathews and Roehrig, 1982, J. Immunol. 129: 2763), punta toro virus (Dalrymple et al., 1981, in Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115: 20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), antigen of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

The antigenic or immunogenic agent in the immunogenic composition of this invention may also be a cancer antigen or a tumor antigen. Any cancer or tumor antigen known to one skilled in the art may be used in accordance with the immunogenic compositions of the invention including, but not limited to, KS 1/4 pan-carcinoma antigen (Perez and Walker, 1990, J. Immunol. 142:3662-3667; Bumal, 1988, Hybridoma 7(4):407-415), ovarian carcinoma antigen (CA125) (Yu et al., 1991, Cancer Res. 51(2):468-475), prostatic acid phosphate (Tailor et al., 1990, Nucl. Acids Res. 18(16):4928), prostate specific antigen (Henttu and Vihko, 1989, Biochem. Biophys. Res. Comm. 160(2):903-910; Israeli et al., 1993, Cancer Res. 53:227-230), melanoma-associated antigen p97 (Estin et al., 1989, J. Natl. Cancer Instit. 81(6):445-446), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-63; Mittelman et al., 1990, J. Clin. Invest. 86:2136-2144), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, colorectal tumor-associated antigens such as: CEA, TAG-72 (Yokata et al., 1992, Cancer Res. 52:3402-3408), C017-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immunospecifically. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos. In one embodiment, the antigen is a T cell receptor derived peptide from a Cutaneous T cell Lymphoma (see, Edelson, 1998, The Cancer Journal 4:62).

The antigenic or immunogenic agent in the immunogenic composition of this invention may comprise a virus, against which an immune response is desired. In certain cases, the immunogenic composition of this invention comprise recombinant or chimeric viruses. In other cases, the immunogenic composition of this invention comprises a virus which is attenuated. Production of recombinant, chimeric and attenuated viruses may be performed using standard methods known to one skilled in the art. This invention also encompasses a live recombinant viral vaccine or an inactivated recombinant viral vaccine to be formulated in accordance with the invention. A live vaccine may be preferred because multiplication in the host leads to a prolonged stimulus of similar kind and magnitude to that occurring in natural infections, and therefore, confers substantial, long-lasting immunity. Production of such live recombinant virus vaccine formulations may be accomplished using conventional methods involving propagation of the virus in cell culture or in the allantois of the chick embryo followed by purification.

The recombinant virus may be non-pathogenic to the subject to which it is administered. In this regard, the use of genetically engineered viruses for vaccine purposes may require the presence of attenuation characteristics in these strains. The introduction of appropriate mutations (e.g., deletions) into the templates used for transfection may provide the novel viruses with attenuation characteristics. For example, specific missense mutations which are associated with temperature sensitivity or cold adaptation can be made into deletion mutations. These mutations should be more stable than the point mutations associated with cold or temperature sensitive mutants and reversion frequencies should be extremely low.

Alternatively, chimeric viruses with “suicide” characteristics may be constructed for use in the composition of this invention. Such viruses would go through only one or a few rounds of replication within the host. When used as a vaccine, the recombinant virus would go through limited replication cycle(s) and induce a sufficient level of immune response but it would not go further in the human host and cause disease.

Alternatively, inactivated (killed) virus may be formulated in accordance with the invention. Inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or β-propiolactone, and pooled.

Completely foreign epitopes, including antigens derived from other viral or non-viral pathogens can also be engineered into the virus for use in the composition of this invention. For example, antigens of non-related viruses such as HIV (gp160, gp120, gp41) parasite antigens (e.g., malaria), bacterial or fungal antigens or tumor antigens can be engineered into the attenuated strain. Methods for production and manufacturing of vaccines are known to one skilled in the art and encompassed within the instant invention. Typically such methods include inoculating embryonated eggs, harvesting the allantoic fluid, concentrating, purifying and separating the whole virus, using for example zonal centrifugation, ultracentrifugation, ultrafiltration, and chromatography in a variety of combinations. Such methods encompass use of various chemicals for example as splitting agents (e.g., non-ionic surfactants, bile acids and derivatives thereof, alkyglycosides and derivatives thereof, acyl sugars), stabilizers, solvents, etc. In such cases, residual concentrations of these chemicals may be found in the final immunogenic composition, left over from the manufacturing and preparation of the vaccine compositions, however, such residual concentrations are not sufficient to result in an adjuvant activity of the vaccine compositions when it is delivered to the intradermal compartment of a subject's skin. It should be emphasized that the concentration of the excipients of the invention as specified herein is greater than the residual concentration of such chemicals that may be present during the preparation and manufacturing of a vaccine composition.

Virtually any heterologous gene sequence may be constructed into the chimeric viruses for use in the immunogenic composition of this invention. Preferably, heterologous gene sequences are moieties and peptides that act as biological response modifiers. Preferably, epitopes that induce a protective immune response to any of a variety of pathogens, or antigens that bind neutralizing antibodies may be expressed by or as part of the chimeric viruses. For example, heterologous gene sequences that can be constructed into the chimeric viruses include, but are not limited to, influenza and parainfluenza hemagglutinin neuraminidase and fusion glycoproteins such as the HN and F genes of human PIV3. In addition, heterologous gene sequences that can be engineered into the chimeric viruses include those that encode proteins with immuno-modulating activities. Examples of immuno-modulating proteins include, but are not limited to, cytokines, interferon type 1, gamma interferon, colony stimulating factors, interleukin-1, -2, -4, -5, -6, -12, and antagonists of these agents.

Other heterologous sequences may be derived from tumor antigens, and the resulting chimeric viruses be used to generate an immune response against the tumor cells leading to tumor regression in vivo. In accordance with the present invention, recombinant viruses may be engineered to express tumor-associated antigens (TAAs), including but not limited to, human tumor antigens recognized by T cells (Robbins and Kawakami, 1996, Curr. Opin. Immunol. 8:628-636, incorporated herein by reference in its entirety); melanocyte lineage proteins, including gp100, MART-1/MelanA, TRP-1 (gp75) and tyrosinase; tumor-specific widely shared antigens, such as MAGE-1, MAGE-3, BAGE, GAGE-1, GAGE-1, N-acetylglucosaminyltransferase-V and p15; tumor-specific mutated antigens, such as β-catenin, MUM-1 and CDK4; non-melanoma antigens for breast, ovarian, cervical and pancreatic carcinoma, HER-2/neu, human papillomavirus-E6, -E7, MUC-1.

The antigenic or immunogenic agent for use in the immunogenic composition of this invention may include one or more of the select agents and toxins as identified by the Center for Disease Control. In certain cases, the select agent for use in the immunogenic composition of this invention may comprise one or more antigens from Staphyloccocal enterotoxin B, Botulinum toxin, protective antigen for Anthrax, and Yersinia pestis. A non-limiting examples of select agents and toxins for use in the immunogenic composition of this invention are listed in Table I:

TABLE I
SELECT AGENTS

HHS NON-OVERLAP SELECT AGENTS AND TOXINS
Crimean-Congo haemorrhagic fever virus
Coccidioides posadasii
Ebola viruses
Cercopithecine herpesvirus 1 (Herpes B virus)
Lassa fever virus
Marburg virus
Monkeypox virus
Rickettsia prowazekii
Rickettsia rickettsii
South American haemorrhagic fever viruses
Junin
Machupo
Sabia
Flexal
Guanarito
Tick-borne encephalitis complex (flavi) viruses
Central European tick-borne encephalitis
Far Eastern tick-borne encephalitis
Russian spring and summer encephalitis
Kyasanur forest disease
Omsk hemorrhagic fever
Variola major virus (Smallpox virus)
Variola minor virus (Alastrim)
Yersinia pestis
Abrin
Conotoxins
Diacetoxyscirpenol
Ricin
Saxitoxin
Shiga-like ribosome inactivating proteins
Tetrodotoxin
HIGH CONSEQUENCE LIVESTOCK PATHOGENS AND
TOXINS/SELECT AGENTS (OVERLAP AGENTS)
Bacillus anthracis
Brucella abortus
Brucella melitensis
Brucella suis
Burkholderia mallei
(formerly Pseuodomonas mallei)
Burkholderia pseudomallei
(formerly Pseuodomonas pseudomallei)
Botulinum neurotoxin producing species of Clostridium
Coccidioides immitis
Coxiella burnetii
Eastern equine encephalitis virus
Hendra virus
Francisella tularensis
Nipah Virus
Rift Valley fever virus
Venezuelan equine encephalitis virus
Botulinum neurotoxin
Clostridium perfringens epsilon toxin
Shigatoxin
Staphylococcal enterotoxin
T-2 toxin
USDA HIGH CONSEQUENCE LIVESTOCK
PATHOGENS AND TOXINS (NON-
OVERLAP AGENTS AND TOXINS
Akabane virus
African swine fever virus
African horse sickness virus
Avian influenza virus (highly pathogenic)
Blue tongue virus (Exotic)
Bovine spongiform encephalopathy agent
Camel pox virus
Classical swine fever virus
Cowdria ruminantium (Heartwater)
Foot and mouth disease virus
Goat pox virus
Lumpy skin disease virus
Japanese encephalitis virus
Malignant catarrhal fever virus (Exotic)
Menangle virus
Mycoplasma capricolumi M.F38/M. mycoides
capri
Mycoplasm mycoides mycoides
Newcastle disease virus (VVND)
Peste Des Petits Ruminants virus
Rinderpest virus
Sheep pox virus
Swine vesicular disease virus
Vesicular stomatitis virus (Exotic)
LISTED PLANT PATHOGENS
Liberobacter africanus
Liberobacter asiaticus
Peronosclerospora phillippinensis
Phakopsora pachyrhizi
Plum Pox Potyvirus
Ralstonia solanacearum race 3, biovar 2
Schlerophthora rayssiae var zeae
Synchytrium endobioticum
Xanthomonas oryzae
Xylella fastidiosa (citrus variegated chlorosis
strain)

5.1.3 Influenza Virus Antigens

Preferred vaccine delivery systems of the invention for intradermal delivery are influenza virus vaccines, which may comprise one or more influenza virus antigens. Preferably, the influenza virus antigens used in the intradermal vaccine formulations of the invention are surface antigens, including, but not limited to, haemagglutinin and neuraminidase antigens or a combination thereof. The influenza virus antigens may form part of a whole influenza vaccine formulations. Alternatively, the influenza virus antigens can be present as purified or substantially purified antigens. Techniques for isolating and purifying influenza virus antigens are known to one skilled in the art and are contemplated in the present invention. An example of a haemagglutinin/neuraminidase preparation suitable for use in the compositions of the present invention is the “Fluvirin” product manufactured and sold by Evans Medical Limited of Speke, Merseyside, United Kingdom, and see also S. Renfrey and A. Watts, 1994 Vaccine, 12(8): 747-752; which is incorporated herein by reference in its entirety.

The influenza vaccines useful in the intradermal vaccine formulations of the present invention may be any commercially available influenza vaccine, preferably a trivalent subunit vaccine, e.g., FLUZONE™ attenuated flu vaccine, Aventis Pasteur, Inc. Swiftwater, Pa.). In preferred embodiments, an equivalent therapeutic effect is achieved by delivering an influenza vaccine to the intradermal compartment with lower than the conventional dose used for intramuscular delivery of influenza vaccines. Influenza vaccine formulations of the invention comprise an excipient as disclosed herein or identified by the methods of the invention. When such formulations are delivered to the intradermal compartment, they result in a higher antibody titre relative to conventional modes of delivery or in the absence of an excipient. In some embodiments, the influenza vaccine formulations of the invention result in a 2-fold, 3-fold, 4-fold, 5-fold, or 10-fold enhancement in antibody titre relative to conventional modes of delivery or relative to the absence of the excipient. In a specific embodiment, when comparing equal amounts of Fluzone delivered to the intradermal compartment, Fluzone supplemented with sorbitol results in a serum titer 3× that achieved when Fluzone is administered without sorbitol (See FIG. 12). Although not intending to be bound by any mechanism of action, such adjuvant driven enhancements provide an option to reduce the concentration of the immunogen, accordingly, the amount of immunogen can be reduced by enhancement of the immune response. In some embodiments, the amount of immunogen is reduced by at least 20%, at least 30%, at least 40%, or at least 50%.

The influenza vaccine used in the invention may be a non-live influenza antigenic preparation, preferably a split influenza or a subunit antigenic preparation, prepared using common methods known in the art. Most preferably, the influenza vaccine used in accordance with the invention is a trivalent vaccine. The invention encompasses influenza vaccine formulations comprising a non-live influenza antigenic preparation, preferably a split influenza preparation or a subunit antigenic preparation prepared from a live virus. Most preferably the influenza antigenic preparation is a split influenza antigenic preparation.

The influenza vaccine formulation of the invention may contain influenza virus antigens from a single viral strain, or from a plurality of strains. For example, the influenza vaccine formulation may contain antigens taken from up to three or more viral strains. Purely by way of example the influenza vaccine formulation may contain antigens from one or more strains of influenza A together with antigens from one or more strains of influenza B. Examples of influenza strains are strains of influenza A/Texas/36/91, A/Nanchang/933/95 and B/Harbin/7/94).

In a most preferred embodiment, the influenza vaccine formulation of the invention comprises a commercially available influenza vaccine, FLUZONE™, which is an attenuated flu vaccine (Connaught Laboratories, Swiftwater, Pa.). FLUZONE™ is a trivalent subvirion vaccine comprising 15 μg/dose of each the HAs from influenza A/Texas/36/91 (NINI), A/Beijing/32/92 (H3N2) and B/Panama, 45/90 viruses.

Preferably, the influenza vaccine formulations of the invention have a lower quantity of haemagglutinin than conventional vaccines and are administered in a lower volume. In some embodiments, the quantity of haemagglutinin per strain of influenza is about 1-7.5 μg. more preferably approximately 3 μg or approximately 5 μg, which is about one fifth or one third, respectively, of the dose of haemagglutinin used in conventional vaccines for intramuscular administration.

The volume of a dose of an influenza vaccine formulation according to the invention is between 0.025 mL and 1.0 mL, more preferably approximately 0.05 mL or approximately 0.25 mL. In a specific embodiment, the invention encompasses a 100 μL dose volume of the influenza vaccine. A 0.1 mL dose is approximately one fifth of the volume of a conventional intramuscular flu vaccine dose. The volume of liquid that can be administered intradermally depends in part upon the site of the injection. For example, for an injection in the deltoid region, 0.1 mL is the maximum preferred volume whereas in the lumbar region a large volume e.g. about 0.2 mL can be given.

Standards are applied internationally to measure the efficacy of influenza vaccines. The European Union official criteria for an effective vaccine against influenza are set out in the table below. Theoretically, to meet the European Union requirements, and thus be approved for sale in the EU, an influenza vaccine has to meet one of the criteria in the table below, for all strains of influenza included in the vaccine. However in practice, at least two or more, probably all three of the criteria will need to be met for all strains, particularly for a new vaccine coming onto the market. Under some circumstances, two criteria may be sufficient. For example, it may be acceptable for two of the three criteria to be met by all strains while the third criterion is met by some but not all strains (e.g. two out of three strains). The requirements are different for adult populations (18-60 years) and elderly populations (>60 years).

TABLE II
EU STANDARDS FOR AN EFFECTIVE INFLUENZA VACCINE

| | 18-60 years | >60 years |
|---|---|---|
| Seroconversion rate | >40% | >30% |
| Conversion factor | >2.5 | >2.0 |
| Protection rate | >70% | >60% |

Seroconversion rate is defined as the percentage of recipients who have at least a 4-fold increase in serum haemagglutinin inhibition (HI) titers after vaccination, for each vaccine strain. Conversion factor is defined as the fold increase in serum HI geometric mean titers after vaccination, for each vaccine strain. Protection rate or seroprotection rate is defined as the percentage of recipients with a serum HI titer equal to or greater than 1:40 after vaccination and is normally accepted as indicating protection.

The influenza vaccine formulations of the invention meet some or all of the EU criteria for influenza vaccines as set out hereinabove, such that the vaccine is approvable in Europe. Preferably, at least two out of the three EU criteria are met, for the or all strains of influenza represented in the vaccine. More preferably, at least two criteria are met for all strains and the third criterion is met by all strains or at least by all but one of the strains. More preferably, all strains present meet all three of the criteria. Preferably, the influenza vaccine formulations of the invention additionally meet some or all criteria of the Federal Drug Administration and/or USPHS requirements for the current influenza vaccines.

5.2 Preparation of the Immunogenic Composition

5.2.1 Preparation of Intradermal Immunogenic Composition

The immunogenic composition of this invention may be prepared by any method that results in a stable, sterile, injectable formulation. Preferably, the method for preparing an immunogenic composition of this invention comprises: providing a solution of the excipient; providing a solution of the antigenic or immunogenic agent; and combining the solution of the excipient and the solution of the antigenic or immunogenic agent to form the inoculum, e.g., the solution to be injected to the intradermal compartment.

In one embodiment, the excipient, in a particulate form, may be dissolved in a solution of the antigenic or immunogenic agent, such that a stable, sterile, injectable formulation is formed. Alternatively, the antigenic or immunogenic agent may be particulate and dissolved in the excipient solution such that a stable, sterile, injectable formulation is formed. For enhanced performance of the immunogenic composition of this invention, the antigenic or immunogenic agent should be uniformly dispersed throughout the composition.

In one embodiment, the excipient and the antigenic or immunogenic agent are mixed prior to administration to a subject. Alternatively, the excipient and the antigenic or immunogenic agent can be mixed during administration in a delivery device.

The amount of the antigenic or immunogenic agent used in the immunogenic composition of this invention may vary depending on the chemical nature and the potency of the antigenic or immunogenic agent and the specific excipient used. Typically, the starting concentration of the antigenic or immunogenic agent in the composition of this invention is the amount that is conventionally used for eliciting the desired immune response, using the conventional routes of administration, e.g., intramuscular injection. The concentration of the antigenic or immunogenic agent is then adjusted, e.g., by dilution using a diluent, in the intradermal vaccine formulations of the invention so that an effective protective immune response is achieved as assessed using standard methods known in the art and described herein.

The amount of the excipient used in the immunogenic composition of this invention may vary depending on the chemical nature of the excipient and the specific antigenic or immunogenic agent used. Certain preferred concentrations of the excipients, described in Section 5.1.1, above, can generally be used effectively with many antigenic or immunogenic agent. One of ordinary skill in the art would appreciate, however, that depending on the individual excipient and the antigenic or immunogenic agent, the amount of excipient may be adjusted using the methods that are substantially identical to those disclosed above for the determination of an effective amount of the antigenic or immunogenic agent, as well as other methods conventionally known in the art.

The immunogenic compositions of the present invention can be prepared as unit dosage forms. A unit dosage per vial may contain 0.1 mL to 1 mL, preferably 0.1 to 0.5 mL of the formulation. In some embodiments, a unit dosage form of the immunogenic compositions of the invention may contain 50 μL to 100 μL, 150 μL to 200 μL, or 250 μL to 500 μL of the formulation. If necessary, these preparations can be adjusted to a desired concentration by adding a sterile diluent to each vial. The immunogenic compositions of the invention are more effective in eliciting the desired immune response, and thus the total volume for intradermal delivery may be less than the volume that is conventionally used.

In some embodiments, the components of the immunogenic compositions of the invention, e.g., the antigenic or immunogenic agent and the excipient, are supplied either separately or mixed together in unit dosage form, for example, as a dry lyophilized powder or water free concentrate in a hermetically sealed container such as an ampoule or a sachette indicating the quantity of the active agent, e.g., the antigenic or immunogenic agent. In other embodiments, an ampoule of sterile diluent can be provided so that the components may be mixed prior to administration. In a specific embodiment, the excipient may be mixed with the antigenic or immunogenic agent just prior to administration. In another specific embodiment, the excipient may be mixed with the antigenic or immunogenic agent in an intradermal delivery device during administration.

The invention also provides immunogenic compositions that are packaged in a hermetically sealed container such as an ampoule or a sachette indicating the quantity of the components. In one embodiment, the immunogenic composition is supplied as a liquid, in another embodiment, as a dry sterilized lyophilized powder or water free concentrate in a hermetically sealed container and can be reconstituted, e.g., with water or saline to the appropriate concentration for administration to a subject. In an alternative embodiment, the immunogenic composition is supplied in liquid form in a hermetically sealed container indicating the quantity and concentration of the components. The immunogenic composition of the invention may be prepared by any method that results in a stable, sterile, injectable formulation.

The immunogenic compositions of the invention have particular utility for intradermal delivery of the antigenic or immunogenic agent to the intradermal compartment of a subject's skin. Preferably, the immunogenic compositions of the invention are administered using any of the intradermal devices and methods disclosed in U.S. patent application Ser. Nos. 09/417,671, filed on Oct. 14, 1999; 09/606,909, filed on Jun. 29, 2000; 09/893,746, filed on Jun. 29, 2001; 10/028,989, filed on Dec. 28, 2001; 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety.

The immunogenic compositions of the invention are administered to the intradermal compartment of a subject's skin such that the intradermal space of the subject's skin is penetrated, without passing through it. Preferably, the immunogenic compositions are administered to the intradermal space at a depth of about 1.0 to 3.0 mm, most preferably at a depth of 1.0 to 2.0 mm. The immunogenic compositions of the invention for intradermal delivery provide a pain-free and less invasive mode of administration as compared to conventional modes of administrations, e.g., i.m., for vaccine formulations, and therefore are more advantageous, for example, in terms of the subjects' compliance.

In some embodiments, the immunogenic compositions of the invention are administered within 12 hours, preferably within 6 hours, within 5 hours, within 3 hours, or within 1 hour after preparation, for example, after being reconstituted from the lyophilized powder. In a preferred embodiment, the immunogenic compositions of the invention are prepared for intradermal administration into a subject immediately prior to the intradermal administration, i.e., the antigenic or immunogenic agent is mixed with the excipient.

The immunogenic compositions of the invention have little or no short term and/or long term toxicity when administered in accordance with the methods of the invention. In some embodiments, the immunogenic compositions of the invention when intradermally administered have an undesired reaction at the site of the injection, e.g., skin irritation, swelling, rash, necrosis, skin sensitization. In these particular embodiments, one or more other excipients are used in the immunogenic compositions of the invention other than the excipient already used, which results in eliminating or reducing the undesired reaction at the site of injection. In other embodiments, the immunogenic compositions of the invention when intradermally administered have no undesired reaction at the site of the injection.

5.2.2 Preparation of Epidermal Immunogenic Composition

The epidermal immunogenic compositions of the invention may be prepared by any method that results in a stable, sterile formulation such as those known in the art and disclosed in U.S. Provisional patent application Nos. 60/330,713, 60/333,162 and U.S. application Ser. No. 09/576,643, U.S. application Ser. No. 10/282,231, filed Oct. 29, 2001, Nov. 27, 2001, and May 22, 2000 and Oct. 29, 2002, respectively, all of which are each hereby incorporated by reference in their entirety. They can be delivered, inter alia, in the form of dry powders, gels, solutions, suspensions, and creams.

The epidermal immunogenic compositions may be delivered into the epidermal compartment of skin in any pharmaceutically acceptable form. In one embodiment the epidermal immunogenic composition is applied to the skin and an abrading device is then moved or rubbed reciprocally over the skin and the substance. It is preferred that the minimum amount of abrasion to produce the desired result be used. Determination of the appropriate amount of abrasion for a selected composition is within the ordinary skill in the art. In another embodiment the immunogenic composition may be applied in dry form to the abrading surface of the delivery device prior to application. In this embodiment, a reconstituting liquid is applied to the skin at the delivery site and the formulation-coated abrading device is applied to the skin at the site of the reconstituting liquid. It is then moved or rubbed reciprocally over the skin so that the immunogenic composition becomes dissolved in the reconstituting liquid on the surface of the skin and is delivered simultaneously with abrasion. Alternatively, a reconstituting liquid may be contained in the abrading device and released to dissolve the immunogenic composition as the device is applied to the skin for abrasion. It has been found that certain vaccine formulations, may also be coated on the abrading device in the form of a gel.

5.3 Administration of the Immunogenic Compositions

5.3.1 Intradermal Administration Methods

The invention encompasses methods for intradermal delivery of the immunogenic compositions of the invention described and exemplified herein to the intradermal compartment of a subject's skin, preferably by directly and selectively targeting the intradermal compartment. Once the immunogenic composition is prepared in accordance to the methods described in Section 5.2, above, the inoculum is typically transferred to an injection device for intradermal delivery, e.g., a syringe. Preferably, the inoculum is administered to the intradermal compartment of a subject's skin within 1 hour of preparation. The immunogenic compositions of the invention are administered using any of the intradermal devices and methods disclosed in U.S. patent application Ser. No. 09/417,671, filed on Oct. 14, 1999; 09/606,909, filed on Jun. 29, 2000; 09/893,746, filed on Jun. 29, 2001; 10/028,989, filed on Dec. 28, 2001; 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety. Exemplary devices are shown in FIGS. 12-14.

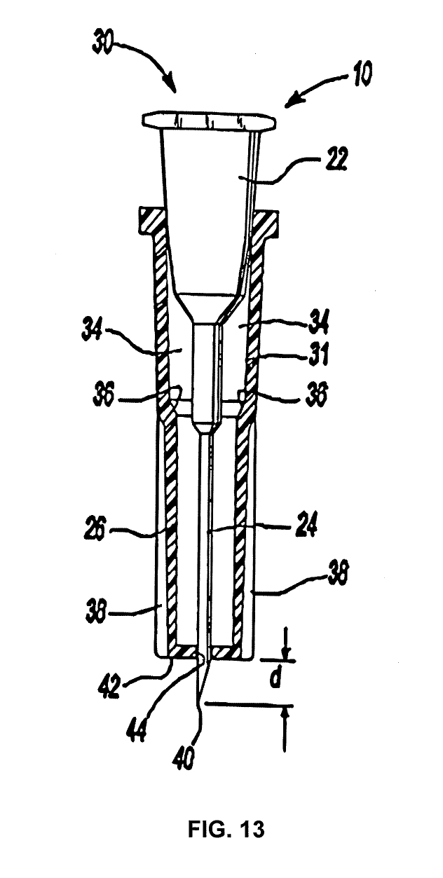

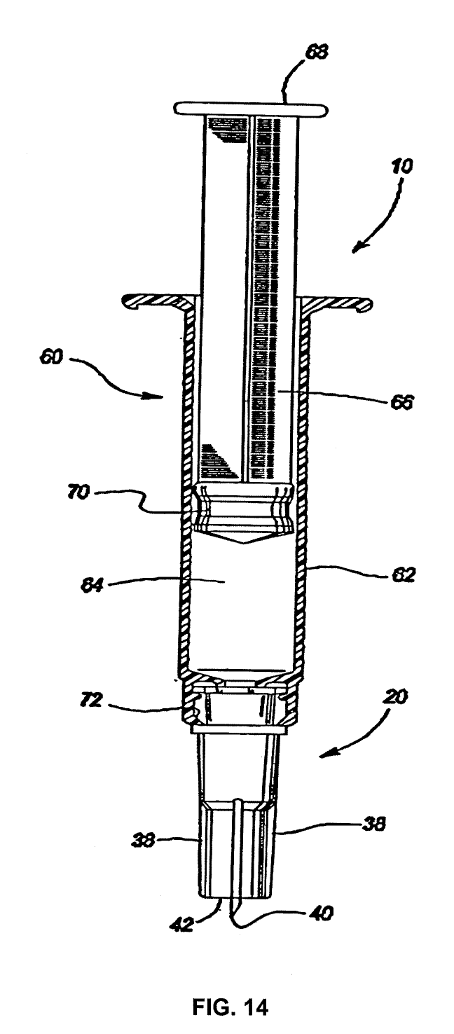

In a specific embodiment, the invention encompasses a drug delivery device as disclosed in FIGS. 12-14. FIGS. 12-14 illustrate an example of a drug delivery device which can be used to practice the methods of the present invention for making intradermal injections illustrated in FIGS. 12-14. The device 10 illustrated in FIGS. 12-14 includes a needle assembly 20 which can be attached to a syringe barrel 60. Other forms of delivery devices may be used including pens of the types disclosed in U.S. Pat. No. 5,279,586, U.S. patent application Ser. No. 09/027,607 and PCT Application No. WO 00/09135, the disclosure of which are hereby incorporated by reference in their entirety. The needle assembly 20 includes a hub 22 that supports a needle cannula 24. The limiter 26 receives at least a portion of the hub 22 so that the limiter 26 generally surrounds the needle cannula 24 as best seen in FIG. 13.

One end 30 of the hub 22 is able to be secured to a receiver 32 of a syringe. A variety of syringe types for containing the substance to be intradermally delivered according to the present invention can be used with a needle assembly designed, with several examples being given below. The opposite end of the hub 22 preferably includes extensions 34 that are received against abutment surfaces 36 within the limiter 26. A plurality of ribs 38 preferably are provided on the limiter 26 to provide structural integrity and to facilitate handling the needle assembly 20. By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of an animal. Typically, the outer skin layer, epidermis, has a thickness between 50-200 microns, and the dermis, the inner and thicker layer of the skin, has a thickness between 1.5-3.5 mm. Below the dermis layer is subcutaneous tissue (also sometimes referred to as the hypodermis layer) and muscle tissue, in that order.

As can be best seen in FIG. 13, the limiter 26 includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against an animal's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the subject's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

It is important to note that although FIGS. 12 and 13 illustrate a two-piece assembly where the hub 22 is made separate from the limiter 26, a device for use in connection with the invention is not limited to such an arrangement. Forming the hub 22 and limiter 26 integrally from a single piece of plastic material is an alternative to the example shown in FIGS. 12 and 13. Additionally, it is possible to adhesively or otherwise secure the hub 22 to the limiter 26 in the position illustrated in FIG. 12 so that the needle assembly 20 becomes a single piece unit upon assembly.

Having a hub 22 and limiter 26 provides the advantage of making an intradermal needle practical to manufacture. The preferred needle size is a small Gauge hypodermic needle, commonly known as a 30 Gauge or 31 Gauge needle. Having such a small diameter needle presents a challenge to make a needle short enough to prevent undue penetration beyond the dermis layer of an animal. The limiter 26 and the hub 22 facilitate utilizing a needle 24 that has an overall length that is much greater than the effective length of the needle, which penetrates the individual's tissue during an injection. With a needle assembly designed in accordance herewith, manufacturing is enhanced because larger length needles can be handled during the manufacturing and assembly processes while still obtaining the advantages of having a short needle for purposes of completing an intradermal injection.

FIG. 13 illustrates the needle assembly 20 secured to a drug container such as a syringe 60 to form the device 10. A generally cylindrical syringe body 62 can be made of plastic or glass as is known in the art. The syringe body 62 provides a reservoir 64 for containing the substance to be administered during an injection. A plunger rod 66 has a manual activation flange 68 at one end with a stopper 70 at an opposite end as known in the art. Manual movement of the plunger rod 66 through the reservoir 64 forces the substance within the reservoir 64 to be expelled out of the end 40 of the needle as desired.

The hub 22 can be secured to the syringe body 62 in a variety of known manners. In one example, an interference fit is provided between the interior of the hub 22 and the exterior of the outlet port portion 72 of the syringe body 62. In another example, a conventional Luer fit arrangement is provided to secure the hub 22 on the end of the syringe 60. As can be appreciated from FIG. 14, such needle assembly designed is readily adaptable to a wide variety of conventional syringe styles.

The present invention improves the clinical utility and therapeutic efficacy of immunogenic compositions described herein by specifically and selectively, preferably directly, targeting the intradermal space. The immunogenic compositions of the invention may be delivered to the intradermal space as a bolus or by infusion. Apart from the enhancement of the immunogenicity of the compositions of the invention by the excipients of this invention, delivering the immunogenic composition of this invention by selectively targeting the intradermal compartment of a subject's skin improves the availability of the immunogenic or antigenic agent to the immune cells residing in the skin, e.g., antigen presenting cells, in order to effectuate an antigen-specific immune response to the immunogenic composition. Preferably, the methods of the invention, allow for smaller doses of the immunogenic compositions to be administered via the intradermal route.

The intradermal methods of administration comprise microneedle-based injection and infusion systems or any other means to accurately target the intradermal space. The intradermal methods of administration encompass not only microdevice-based injection means, but other delivery methods such as needless or needle-free ballistic injection of fluids or powders into the intradermal space, Mantoux-type intradermal injection, enhanced ionotophoresis through microdevices, and direct deposition of fluid, solids, or other dosing forms into the skin.

The immunogenic composition of this invention may be administered to an intradermal compartment of a subject's skin using an intradermal Mantoux type injection, see, e.g., Flynn et al., 1994, Chest 106: 1463-5, which is incorporated herein by reference in its entirety. Specifically, the immunogenic composition may be delivered to the intradermal compartment of a subject's skin using the following exemplary method. In a specific embodiment, the immunogenic compositions of the invention as prepared in accordance to methods disclosed in Section 5.3, above, is drawn up into a syringe, e.g., a 1 mL latex free syringe with a 20 gauge needle; after the syringe is loaded it is replaced with a 30 gauge needle for intradermal administration. The skin of the subject, e.g., mouse, is approached at the most shallow possible angle with the bevel of the needle pointing upwards, and the skin pulled tight. The injection volume is then pushed in slowly over 5-10 seconds forming the typical “bleb” and the needle is subsequently slowly removed. Preferably, only one injection site is used. More preferably, the injection volume is no more than 100 μL, due in part, to the fact that a larger injection volume may increase the spill over into the surrounding tissue space, e.g., the subcutaneous space.

The invention encompasses the use of conventional injection needles, catheters or microneedles of all known types, employed singularly or in multiple needle arrays. In preferred embodiments, needle arrays are used to deliver larger volumes to the intradermal compartment. For example a larger injection volume, e.g., 500 μL could be divided over several sites simultaneously and thereby allowing more volume to be introduced without exceeding the intradermal compartment. The terms “needle” and “needles” as used herein are intended to encompass all such needle-like structures. The term “microneedles” as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes.

The intradermal delivery of the immunogenic composition of this invention may use ballistic fluid injection devices, powder jet delivery devices, piezoelectric, electromotive, electromagnetic assisted delivery devices, gas-assisted delivery devices, which directly penetrate the skin to directly deliver the vaccine formulations of the invention to the targeted location within the dermal space.

The actual method by which the immunogenic composition of the invention are targeted to the intradermal space is not critical as long as it penetrates the skin of a subject to the desired targeted depth within the intradermal space without passing through it. The actual optimal penetration depth will vary depending on the thickness of the subject's skin. In most cases, skin is penetrated to a depth of about 0.5-2 mm. Regardless of the specific intradermal device and method of delivery, the intradermal delivery preferably targets the immunogenic composition of this invention to a depth of at least 0.3 mm, more preferably at least 0.5 mm up to a depth of no more than 2.0 mm, more preferably no more than 1.7 mm.

In certain cases, the immunogenic compositions are delivered at a targeted depth just under the stratum corneum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhans cells in the dermal space of human skin, delivery would need to encompass, at least, in part, the epidermal tissue depth typically ranging from about 0.025 mm to about 0.2 mm in humans.

In the cases where the immunogenic compositions require systemic circulation, the preferred target depth would be between, at least about 0.4 mm and most preferably, at least about 0.5 mm, up to a depth of no more than about 2.5 mm, more preferably, no more than about 2.0 mm and most preferably, no more than about 1.7 mm.

The intradermal administration methods useful for carrying out the invention include both bolus and infusion delivery of the immunogenic compositions to a subject, preferably a mammal, most preferably a human. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes.

The intradermal delivery of the immunogenic compositions into the intradermal space may occur either passively, without application of the external pressure or other driving means to the vaccine formulations to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. If desired, the rate of delivery of the immunogenic composition of this invention may be variably controlled by the pressure-generating means.

The immunogenic compositions delivered or administered in accordance with the invention include solutions thereof in pharmaceutically acceptable diluents or solvents, suspensions, gels, particulates such as micro- and nanoparticles either suspended or dispersed, as well as in-situ forming vehicles of same.

This invention also encompasses varying the targeted depth of delivery of the immunogenic composition of this invention. The targeted depth of delivery of immunogenic compositions may be controlled manually by the practitioner, or with or without the assistance of an indicator to indicate when the desired depth is reached. Preferably, however, the devices used in accordance with the invention have structural means for controlling skin penetration to the desired depth within the intradermal space. The targeted depth of delivery may be varied using any of the methods described in U.S. patent application Ser. Nos. 09/417,671, filed on Oct. 14, 1999; 09/606,909, filed on Jun. 29, 2000; 09/893,746, filed on Jun. 29, 2001; 10/028,989, filed on Dec. 28, 2001; 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety.

The dosage of the immunogenic composition of this invention depends on the antigenic or immunogenic agent in the composition. The dosage of the immunogenic composition may be determined using standard immunological methods known in the art, for example, by first identifying doses effective to elicit a prophylactic or therapeutic immune response, e.g., by measuring the serum titer of antigen specific immunoglobulins, relative to a control formulation, e.g., a formulation simply consisting of the antigenic or immunogenic agent without an excipient as disclosed herein. Preferably, the effective dose is determined in an animal model, prior to use in humans. Most preferably, the optimal dose is determined in an animal whose skin thickness approximates closely to that of human skin, e.g., pig.

The immunogenic compositions of this invention may also be administered on a dosage schedule, for example, an initial administration of the immunogenic composition with subsequent booster administrations. In certain cases, a second dose of the immunogenic composition is administered anywhere from two weeks to one year, preferably from one to six months, after the initial administration. Additionally, a third dose may be administered after the second dose and from three months to two years, or even longer, preferably 4 to 6 months, or 6 months to one year after the initial administration. In certain cases, no booster immunization is required.

5.3.2 Epidermal Administration

The epidermal methods of administration comprise any method and device known in the art for accurately targeting the epidermal compartment such as those disclosed in U.S. Provisional patent application Nos. 60/330,713, 60/333,162 and U.S. application Ser. No. 09/576,643, U.S. application Ser. No. 10/282,231, filed Oct. 29, 2001, Nov. 27, 2001, and May 22, 2000 and Oct. 29, 2002, respectively, all of which are each hereby incorporated by reference in their entirety. The present invention encompasses micoabrading devices for accurately targeting the epidermal space. These devices may have solid or hollow micro-protrusions. The micro-protrusions can have a length up to about 500 microns. Suitable micro-protrusions have a length of about 50 to 500 microns. Preferably the microprotrusions have a length of about 50 to 300 microns and more preferably in the range of about 150 to 250 microns, with 180 to 220 microns being most preferred.

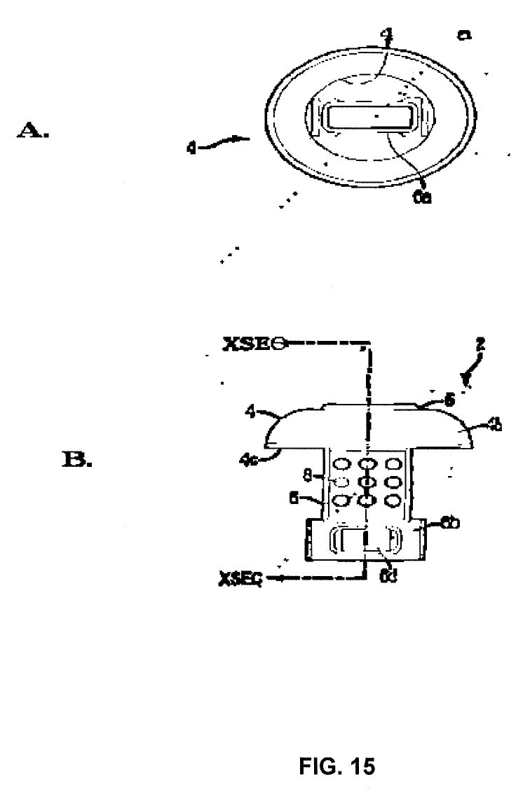

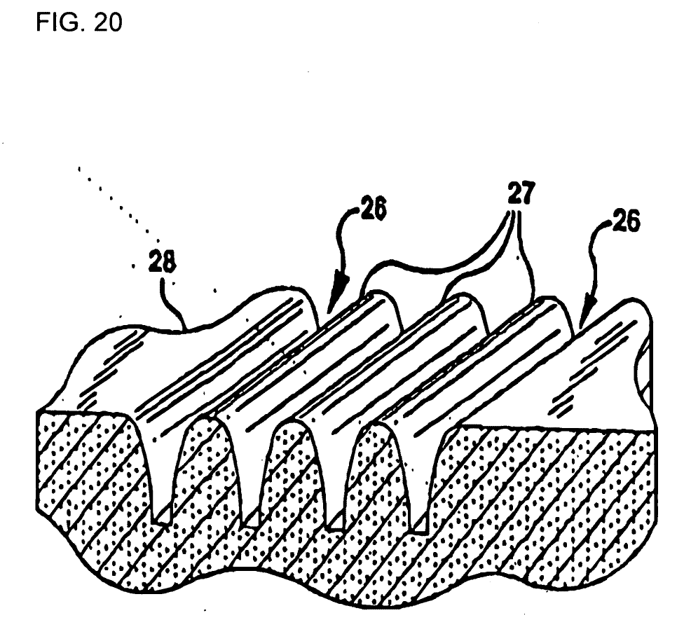

The microabrader devices that may be used in the methods of the invention are preferably a device capable of abrading the skin such as those exemplified in FIGS. 15-20. In preferred embodiments, the device is capable of abrading the skin thereby penetrating the stratum corneum without piercing the stratum corneum.

As used herein, “penetrating” refers to entering the stratum corneum without passing completely through the stratum corneum and entering into the adjacent layers. This is not to say that that the stratum corneum can not be completely penetrated to reveal the interface of the underlying layer of the skin. Piercing, on the other hand, refers to passing through the stratum corneum completely and entering into the adjacent layers below the stratum corneum. As used herein, the term “abrade” refers to removing at least a portion of the stratum corneum to increase the permeability of the skin without causing excessive skin irritation or compromising the skin's barrier to infectious agents. The term “abrasion” as used herein refers to disruption of the outer layers of the skin, for example by scraping or rubbing, resulting in an area of disrupted stratum corneum. This is in contrast to “puncturing” which produces discrete holes through the stratum corneum with areas of undisrupted stratum corneum between the holes.

Preferably, the devices used for epidermal delivery in accordance with the methods of the invention penetrate, but do not pierce, the stratum corneum. The compositions to be administered using the methods of this invention may be applied to the skin prior to abrading, simultaneous with abrading, or post-abrading.

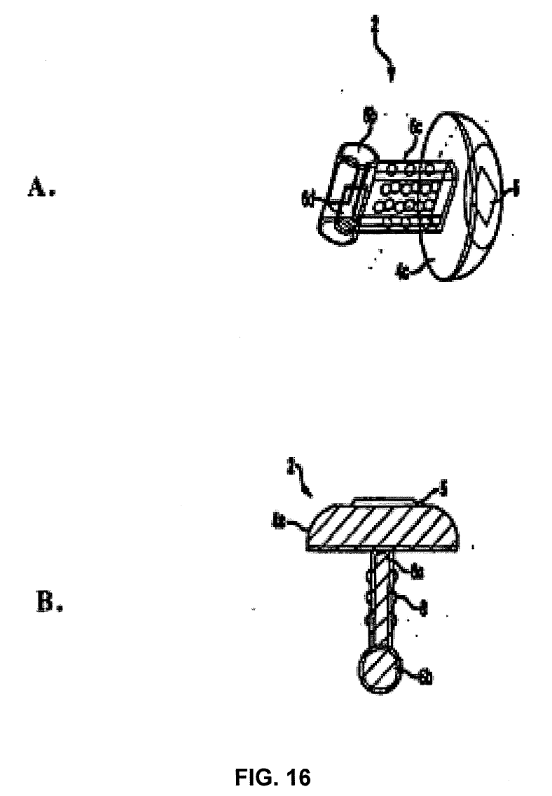

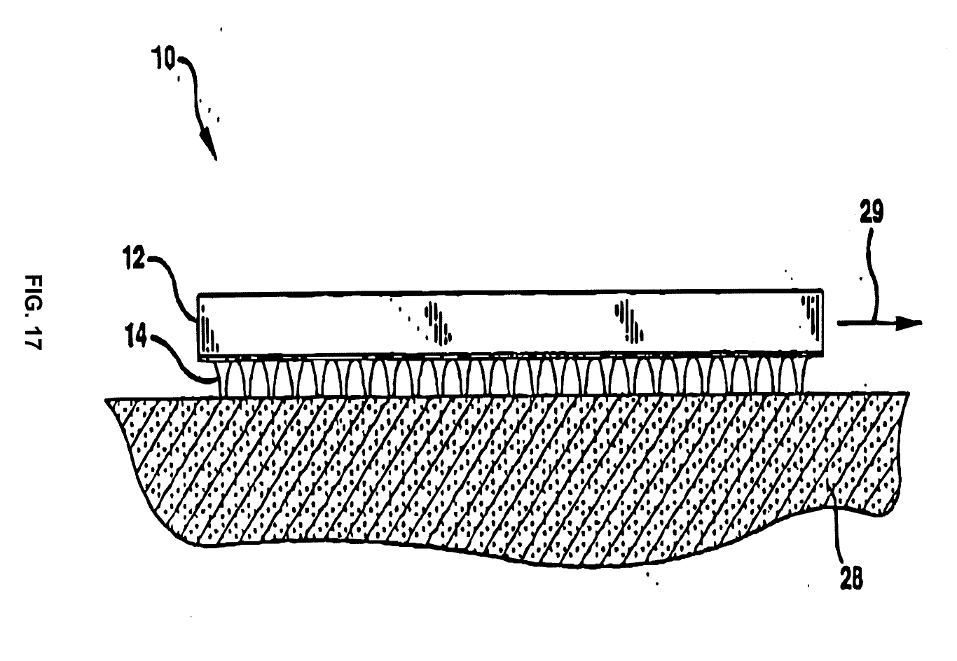

In a specific embodiment the invention encompasses a method for delivering an immunogenic compositions into the skin of a patient comprising the steps of coating a patient's outer skin layer or a microabrader 2, see FIG. 15B with the formulation and moving microabrader 2 across the patient's skin to provide abrasions leaving furrows sufficient to permit entry of the formulation into the patient's viable epidermis. Due to the structural design of microabrader 2, the leading edge of microabrader 2 first stretches the patient's skin and then the top surface of microabrader 2 abrades the outer protective formulation e to enter the patient. After the initial abrasion of the outer protective skin layer, the trailing and leading edges of microabrader 2 can rub the surface of the abraded area working the formulation into the abraded skin area thereby improving its medicinal effect. As shown in FIGS. 15B, 16A and 16B, microabrader 2 includes base 4 onto which an abrading surface 5 can be mounted. Alternatively, the abrading surface may be integral with the base and fabricated as a single two-component part. Preferably, base 4 is a solid molded piece. In one embodiment, base 4 is configured with a mushroom-like crown 4b that curves upward and is truncated at the top. The top of base 4 is generally flat with abrading surface 5 being mounted thereon or integral therewith. Alternatively, the truncated top may have a recess for receiving abrading surface 5. In all embodiments, abrading surface 5 includes a platform with an array of microprotrusions that extends above the truncated top. In another embodiment of the microabrader, the handle, base and abrading surface may be integral with one another and fabricated as a single three-component device. Microabrader 2 is applied to a subject by moving microabrader 2 across the subject's skin with enough pressure to enable abrading surface 5 to open the outer protective skin or stratum corneum of the subject. The inward pressure applied to the base causes microabrader 2 to be pressed into the subject's skin. Accordingly, it is preferable that the height of the sloping mushroom-like crown 4b be sufficient to prevent the applied substance from flowing over and onto the facet 4c when microabrader 2 is being used. As will be described below, abrading surface 5 comprises an array of microprotrusions.

A handle 6 is attached to base 4 or may be integral with base 4. As shown in FIG. 16A, an upper end 6a of the handle may be either snap fit or friction fit between the inner circumferential sidewall 4a of base 4. Alternatively, as shown in FIGS. 15A and 16A, handle 6 may be glued (e.g., with epoxy) to the underside 4c of base 4. Alternatively, the handle and base may be fabricated (e.g., injection-molded) together as a single two-component part. The handle may be of a diameter that is less than the diameter of the base or may be of a similar diameter as the base. Underside 4c of base 4 may be flush with mushroom-like crown 4b or extend beyond the mushroom-like crown. The lower end 6b of handle 6 may be wider than the shaft 6c of handle 6 or may be of a similar diameter as shaft. Lower end 6b may include an impression 6d that serves as a thumb rest for a person administering the substance and moving microabrader 2. In addition, protrusions 8 are formed on the outside of handle 6 to assist a user in firmly gripping handle 6 when moving the same against or across a patient's skin.

As shown in the cross-section of FIG. 15B in FIG. 16B, lower end 6b may be cylindrical. Microabrader 2 may be made of a transparent material, as shown in FIG. 16A. Impressions 6d are disposed on both sides of the cylindrical lower end 6b to assist a person using microabrader 2 to grip the same. That is, the movement of microabrader 2 can be provided by hand or fingers. The handle 6, as well as the base 4, of the microabrader is preferably molded out of plastic or the like material. The microabrader 2 is preferably inexpensively manufactured so that the entire microabrader and abrading surface can be disposed after its use on one patient.

Abrading surface 5 is designed so that when microabrader 2 is moved across a patient's skin, the resultant abrasions penetrate the stratum corneum. Abrading surface 5 may be coated with a formulation desired to be delivered to the patient's viable epidermis.

In order to achieve the desired abrasions, the microabrader 2 should be moved across a patient's skin at least once. The patient's skin may be abraded in alternating directions. The structural design of the microabrader according to the invention enables the formulation to be absorbed more effectively thereby allowing less of the formulation to be applied to a patient's skin or coating abrading surface 5. Abrading surface 5 may be coated with a formulation desired to be delivered to the patient. In one embodiment, the formulation may be a powder disposed on abrading surface 5. In another embodiment, the formulation to be delivered may be applied directly to the patient's skin prior to the application and movement of microabrader 2 on the patient's skin.

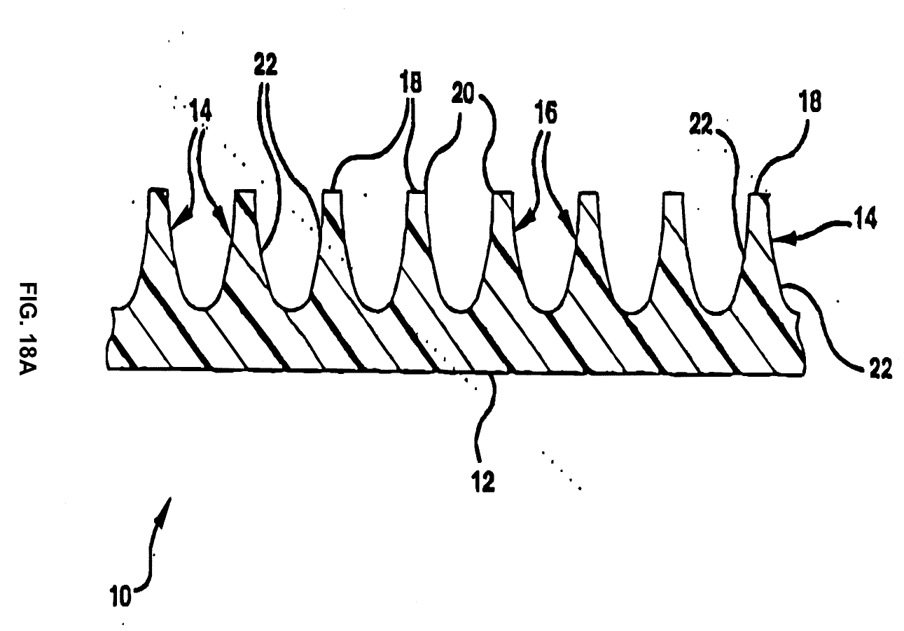

Referring to FIG. 17, the microabrader device 10 of the invention includes a substantially planar body or abrading surface support 12 having a plurality of microprotrusions 14 extending from the bottom surface of the support. The support generally has a thickness sufficient to allow attachment of the surface to the base of the microabrader device thereby allowing the device to be handled easily as shown in FIGS. 15B, 16A and 16B. Alternatively, a differing handle or gripping device can be attached to or be integral with the top surface of the abrading surface support 12. The dimensions of the abrading surface support 12 can vary depending on the length of the microprotrusions, the number of microprotrusions in a given area and the amount of the formulation to be administered to the patient. Typically, the abrading surface support 12 has a surface area of about 1 to 4 cm². In preferred embodiments, the abrading surface support 12 has a surface area of about 1 cm².

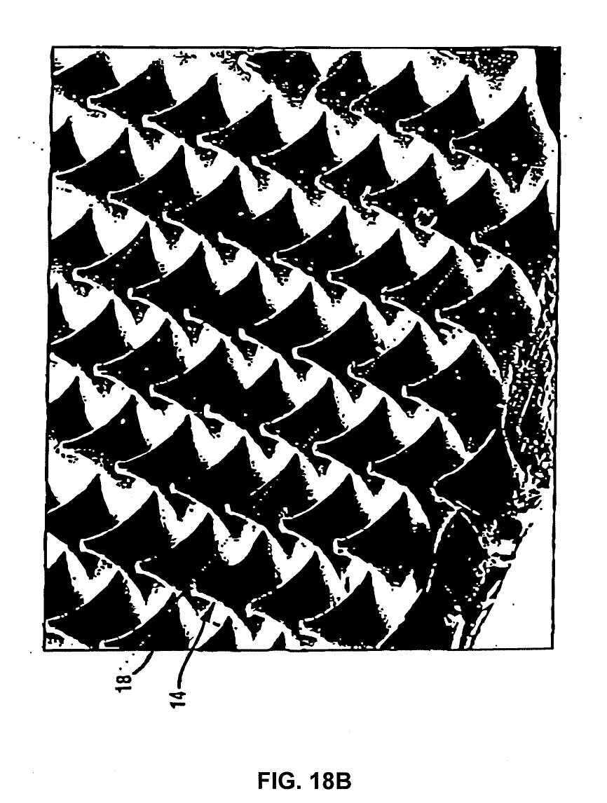

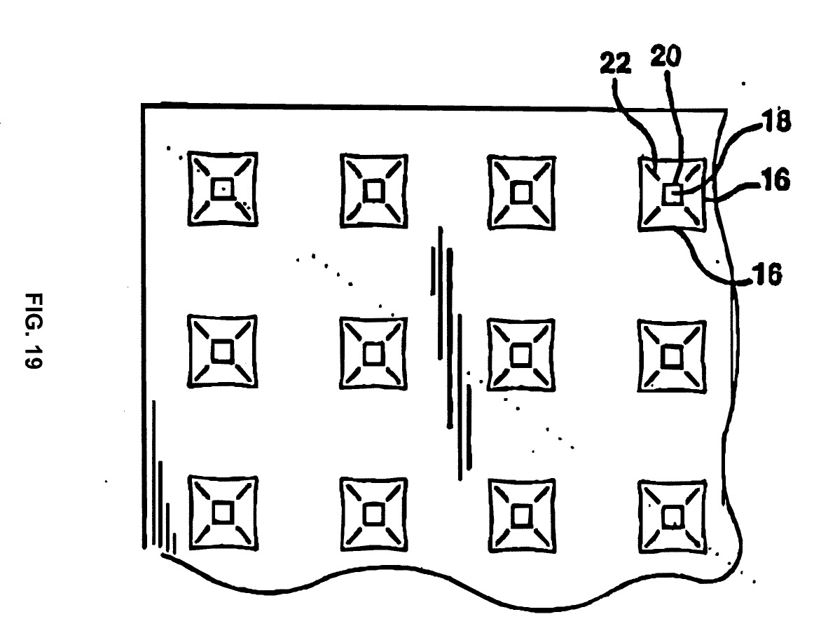

As shown in FIGS. 17, 18A and B and 19, the microprotrusions 14 project from the surface of the abrading surface support 12 and are substantially perpendicular to the plane of the abrading surface support 12. The microprotrusions in the illustrated embodiment are arranged in a plurality of rows and columns and are preferably spaced apart a uniform distance. The microprotrusions 14 have a generally pyramid shape with sides 16 extending to a tip 18. The sides 16 as shown have a generally concave profile when viewed in cross-section and form a curved surface extending from the abrading surface support 12 to the tip 18. In the embodiment illustrated, the microprotrusions are formed by four sides 16 of substantially equal shape and dimension. As shown in FIGS. 18B and 19, each of the sides 16 of the microprotrusions 14 have opposite side edges contiguous with an adjacent side and form a scraping edge 22 extending outward from the abrading surface support 12. The scraping edges 22 define a generally triangular or trapezoidal scraping surface corresponding to the shape of the side 16. In further embodiments, the microprotrusions 14 can be formed with fewer or more sides.

The microprotrusions 14 preferably terminate at blunt tips 18. Generally, the tip 18 is substantially flat and parallel to the support 14. When the tips are flat, the total length of the microprotrusions do not penetrate the skin; thus, the length of the microprotrusions is greater than the total depth to which said microprotrusions penetrate said skin. The tip 18 preferably forms a well defined, sharp edge 20 where it meets the sides 16. The edge 20 extends substantially parallel to the abrading surface support 12 and defines a further scraping edge. In further embodiments, the edge 20 can be slightly rounded to form a smooth transition from the sides 16 to the tip 18. Preferably, the microprotrusions are frustoconical or frustopyramidal in shape.

The microabrader device 10 and the microprotrusions can be made from a plastic material that is non-reactive with the substance being administered. A non-inclusive list of suitable plastic materials include, for example, polyethylene, polypropylene, polyamides, polystyrenes, polyesters, and polycarbonates as known in the art. Alternatively, the microprotrusions can be made from a metal such as stainless steel, tungsten steel, alloys of nickel, molybdenum, chromium, cobalt, titanium, and alloys thereof, or other materials such as silicon, ceramics and glass polymers. Metal microprotrusions can be manufactured using various techniques similar to photolithographic etching of a silicon wafer or micromachining using a diamond tipped mill as known in the art. The microprotrusions can also be manufactured by photolithographic etching of a silicon wafer using standard techniques as are known in the art. They can also be manufactured in plastic via an injection molding process, as described for example in U.S. application Ser. No. 10/193,317, filed Jul. 12, 2002, which is hereby incorporated by reference.

The length and thickness of the microprotrusions are selected based on the particular substance being administered and the thickness of the stratum corneum in the location where the device is to be applied. Preferably, the microprotrusions penetrate the stratum corneum substantially without piercing or passing through the stratum corneum. The microprotrusions can have a length up to about 500 microns. Suitable microprotrusions have a length of about 50 to 500 microns. Preferably, the microprotrusions have a length of about 50 to about 300 microns, and more preferably in the range of about 150 to 250 microns, with 180 to 220 microns most preferred. The microprotrusions in the illustrated embodiment have a generally pyramidal shape and are perpendicular to the plane of the device. These shapes have particular advantages in insuring that abrasion occurs to the desired depth. In preferred embodiments, the microprotrusions are solid members. In alternative embodiments, the microprotrusions can be hollow.

As shown in FIGS. 16 and 18, the microprotrusions are preferably spaced apart uniformly in rows and columns to form an array for contacting the skin and penetrating the stratum corneum during abrasion. The spacing between the microprotrusions can be varied depending on the substance being administered either on the surface of the skin or within the tissue of the skin. Typically, the rows of microprotrusions are spaced to provide a density of about 2 to about 10 per millimeter (mm). Generally, the rows or columns are spaced apart a distance substantially equal to the spacing of the microprotrusions in the array to provide a microprotrusion density of about 4 to about 100 microprotrusions per mm². In another embodiment, the microprotrusions may be arranged in a circular pattern. In yet another embodiment, the microprotrusions may be arranged in a random pattern. When arranged in columns and rows, the distance between the centers of the microprotrusions is preferably at least twice the length of the microprotrusions. In one preferred embodiment, the distance between the centers of the microprotrusions is twice the length of the microprotrusions 110 microns. Wider spacings are also included, up to 3, 4, 5 and greater multiples of the length of the micoprotrusions. In addition, as noted above, the configuration of the microprotrusions can be such, that the height to the microprotrusions can be greater than the depth into the skin those protrusions will penetrate. The flat upper surface of the frustoconical or frustopyramidal microprotrusions is generally 10 to 100, preferably 30-70, and most preferably 35-50 microns in width.

The method of preparing a delivery site on the skin places the microabrader against the skin 28 of the patient in the desired location. The microabrader is gently pressed against the skin and then moved over or across the skin. The length of the stroke of the microabrader can vary depending on the desired size of the delivery site, defined by the delivery area desired. The dimensions of the delivery site are selected to accomplish the intended result and can vary depending on the substance, and the form of the substance, being delivered. For example, the delivery site can cover a large area for treating a rash or a skin disease. Generally, the microabrader is moved about 2 to 15 centimeters (cm). In some embodiments of the invention, the microabrader is moved to produce an abraded site having a surface area of about 4 cm² to about 300 cm².

The microabrader is then lifted from the skin to expose the abraded area and a suitable delivery device, patch or topical formulation may be applied to the abraded area. Alternatively, the substance to be administered may be applied to the surface of the skin either before, or simultaneously with abrasion.

The extent of the abrasion of the stratum corneum is dependent on the pressure applied during movement and the number of repetitions with the microabrader. In one embodiment, the microabrader is lifted from the skin after making the first pass and placed back onto the starting position in substantially the same place and position. The microabrader is then moved a second time in the same direction and for the same distance. In another embodiment, the microabrader is moved repetitively across the same site in alternating direction without being lifted from the skin after making the first pass. Generally, two or more passes are made with the micro abrader.

In further embodiments, the microabrader can be swiped back and forth, in the same direction only, in a grid-like pattern, a circular pattern, or in some other pattern for a time sufficient to abrade the stratum corneum a suitable depth to enhance the delivery of the desired substance. The linear movement of the microabrader across the skin 28 in one direction removes some of the tissue to form grooves 26, separated by peaks 27 in the skin 28 corresponding to substantially each row of microprotrusions as shown in FIG. 16. The edges 20, 22 and the blunt tip 18 of the microprotrusions provide a scraping or abrading action to remove a portion of the stratum corneum to form a groove or furrow in the skin rather than a simple cutting action. The edges 20 of the blunt tips 18 of the microprotrusions 14 scrape and remove some of the tissue at the bottom of the grooves 26 and allows them to remain open, thereby allowing the substance to enter the grooves for absorption by the body. Preferably, the microprotrusions 14 are of sufficient length to penetrate the stratum corneum and to form grooves 26 having sufficient depth to allow absorption of the substance applied to the abraded area without inducing pain or unnecessary discomfort to the patient. Preferably, the grooves 26 do not pierce but can extend through the stratum corneum. The edges 22 of the pyramid shaped microprotrusions 14 form scraping edges that extend from the abrading surface support 12 to the tip 18. The edges 22 adjacent the abrading surface support 12 form scraping surfaces between the microprotrusions which scrape and abrade the peaks 27 formed by the skin between the grooves 26. The peaks 27 formed between the grooves generally are abraded slightly.

Any device known in the art for disruption of the stratum corneum by abrasion can be used in the methods of the invention. These include for example, microelectromechanical (MEMS) devices with arrays of short microneedles or microprotrusions, sandpaper-like devices, scrapers and the like. The actual method by which the epidermal vaccine formulations of the invention are targeted to the epidermal space is not critical as long as it penetrates the skin of a subject to the desired targeted depth. The microabraiders discussed within initially deposit the inventive formulations to a skin depth of 0.0 to 0.025 mm and preferably not exceeding the statum corneum.

5.4 Determination of Therapeutic Efficacy

The invention encompasses methods for determining the efficacy of immunogenic compositions of the invention using any standard method known in the art or described herein. Assays for determining the efficacy of the immunogenic compositions of the invention may be in vitro based assays or in vivo based assays, including animal based assays. In some embodiments, the invention encompasses detecting and/or quantitating a humoral immune response against the antigenic or immunogenic agent of a composition of the invention in a sample, e.g., serum or mucosal wash, obtained from a subject who has been administered an immunogenic composition of the invention. Preferably, the humoral immune response of the immunogenic compositions of the invention are compared to a control sample obtained from the same subject prior to administration with the inventive formulation or after an individual has been administered a control formulation, e.g., a formulation which simply comprises of the antigenic or immunogenic agent.

The methods of the invention provide fundamental principles and guidelines whereby optimum parameters may be determined for delivering immunogenic compositions to the dermal compartment (including epidermal and intradermal compartments) wherein the excipients have optimum adjuvant properties and the formulations of the invention have enhanced efficacy in comparison to when the same formulation is delivered using conventional modes of delivery, including intramuscular and subcutaneous delivery. The invention provides methods wherein the formulations of the invention have been screened to have optimum concentration ranges for delivery to the optimum depth of the intradermal compartment such that they have adjuvant properties, resulting in one or more of the following properties: minimal to no skin irritation as determined and assessed using conventional modes of analysis of skin reactions using visual methods such as Draize scoring (For a typical draize scoring analysis see table below); minimal to no hemolysis as determined using standard methods known in the art, and enhanced immune response as measured by enhanced seroconversion and/or enhanced antibody titers.

TABLE A
Draize Scoring
Key to interpreting skin reactions - Draize Scoring

| Erythema Score | | Edema Score | |
|---|---|---|---|
| No erythema | 0 | No edema | 0 |
| Slight erythema (barely | 1 | Slight edema (barely | 1 |
| perceptible) | | perceptible) | |
| Well-defined erythema | 2 | Well-defined edema | 2 |
| Moderate to severe | 3 | Moderate to severe | 3 |
| Severe erythema (beet redness | 4 | Sever edema (extending | 4 |
| to administration sight, | | beyond the site | |
| injury by depth | | | |

In some embodiments, the invention encompasses detecting and/or quantitating a humoral immune response against the antigenic or immunogenic agent of the immunogenic composition of this invention in a sample, e.g., serum, obtained from a subject who has been administered an immunogenic composition of this invention. The humoral immune response of the immunogenic composition of this invention is compared to a control sample obtained from the same subject, who has been administered a control formulation, e.g., a formulation which simply comprises of the antigenic or immunogenic agent.

Assays for measuring humoral immune response are well known in the art, e.g., see, Coligan et al., (eds.), 1997, Current Protocols in Immunology, John Wiley and Sons, Inc., Section 2.1. A humoral immune response may be detected and/or quantitated using standard methods known in the art including, but not limited to, an ELISA assay. The humoral immune response may be measured by detecting and/or quantitating the relative amount of an antibody which specifically recognizes an antigenic or immunogenic agent in the sera of a subject who has been treated with an immunogenic composition of this invention relative to the amount of the antibody in an untreated subject. ELISA assays can be used to determine total antibody titers in a sample obtained from a subject treated with a composition of the invention. In other embodiments, ELISA assays may be used to determine the level of specific antibody isotypes and antibodies to neutralizing epitopes using methods known in the art.

ELISA based assays comprise preparing an antigen, coating the well of a 96 well microtiter plate with the antigen, adding test and control samples containing antigen specific antibody, adding a detector antibody specific to the antibody in test and control samples that is conjugated to an enzyme (e.g., horseradish peroxidase or alkaline phosphatase) and incubating for a period of time, and detecting the presence of the antigen with a color yielding substrate. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

In the cases where the immunogenic composition comprises an influenza antigen any method known in the art for the detection and/or quantitation of an antibody response against an influenza antigen is encompassed within the methods of the invention. An exemplary method for determining an influenza antigen directed antibody response may comprise the following: an influenza antigen is used to coat a microtitre plate (Nunc plate); sera from a subject treated with an influenza vaccine formulation of the invention is added to the plate; antisera (containing 2^{nd} antibody) is added to the plate and incubated for a sufficient time to allow a complex to be formed, i.e., a complex between an antibody in the sera and the antisera. The complex is then detected using standard methods in the art. For exemplary assays for measuring an influenza specific antibody response, see, e.g., Newman et al., 1997, Mechanism of Aging & Development, 93: 189-203; Katz et al., 2000, Vaccine, 18: 2177-87; Todd et al., (Brown and Haaheim, eds.), 1998 in Modulation of the Immune Response to Vaccine Antigens, Dev. Biol. Stand. Basel, Karger, 92: 341-51; Kendal et al., 1982, in Concepts and Procedures for Laboratory-based Influenza Surveillance, Atlanta: CDC, B17-35; Rowe et al., 1999, J. Clin. Micro. 37: 937-43; Todd et al., 1997, Vaccine 15: 564-70; WHO Collaborating Centers for Reference and Research on Influenza, in Concepts and Procedures for Laboratory-based Influenza Surveillance, 1982, p. B-23; all of which are incorporated herein by reference in their entirety.

Furthermore, when the vaccine formulation comprises an influenza antigen any method known in the art for the detection and/or quantitation levels of antibody with hemagglutination activity are encompassed within the invention. The hemagglutination inhibition assays are based on the ability of influenza viruses to agglutinate erythrocytes and the ability of specific HA antibodies to inhibit agglutination. Any of the hemagglutination inhibition assays known in the art are encompassed within the methods of the inventions, such as those disclosed in Newman et al., 1997, Mechanism of Aging & Development, 93: 189-203; Kendal et al., 1982, in Concepts and Procedures for Laboratory-based Influenza Surveillance, Atlanta: CDC, B17-35; all of which are incorporated herein by reference in their entirety.

An exemplary hemagglutination inhibition assay comprises the following: sera from subjects treated with an influenza vaccine formulation of the invention are added to microtitre plates; HI-antigenic preparation containing 8 HA units is added to the plates; the mixture is mixed well by gently tapping the plates, and incubated for about 1 hour at 4° C.; erythrocyte suspension, e.g., 0.5% chicken erythrocytes, is added to the micotitre plate and the contents are mixed well by gently tapping the plates; the plates are further incubated at 4° C. until the cell control shows the button of normal settling; controls only contains PBS). Preferably, the serum samples are treated with inhibitors, such as neuraminidase or potassium periodate, to prevent non-specific inhibition of agglutination by serum factors. The HI titer is defined as the dilution factor of the highest dilution of serum that completely inhibits hemagglutination. This is determined by tilting the plates and observing the tear shaped streaming of cells that flow at the same rate as control cells.

The invention encompasses methods for determining the efficacy of the compositions of the invention by measuring cell-mediate immune response. Methods for measuring cell-mediated immune response are known to one skilled in the art and encompassed within the invention. In some embodiments, a T cell immune response may be measured for quantitating the immune response in a subject, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

5.5 Prophylactic and Therapeutic Uses

The invention provides methods of treatment and prophylaxis which involve administering an immunogenic composition of the invention to a subject, preferably a mammal, and most preferably a human for treating, managing or ameliorating symptoms associated with a disease or disorder, especially an infectious disease or cancer. The subject is preferably a mammal such as a non-primate, e.g., cow, pig, horse, cat, dog, rat, mouse and a primate, e.g., a monkey such as a Cynomolgous monkey and a human. In a preferred embodiment, the subject is a human. Preferably, the immunogenic composition of the invention is a vaccine composition.

The invention encompasses a method for immunization and/or stimulating an immune response in a subject comprising intradermal delivery of a single dose of a composition of the invention to a subject, preferably a human. In some embodiments, the invention encompasses one or more booster immunizations. The immunogenic composition of the invention is particularly effective in stimulating and/or up-regulating an antibody response to a level greater than that seen in conventional immunogenic compositions (such as vaccines) and administration schedules. For example, an immunogenic composition of the invention may lead to an antibody response comprising generations of one or more antibody classes, such as IgM, IgG, and/or IgA. Most preferably, the immunogenic compositions of the invention including vaccine formulations stimulate a systemic immune response that protects the subject from at least one pathogen. The immunogenic compositions of the invention including vaccine compositions may provide systemic, local, or mucosal immunity or a combination thereof.

5.5.1 Target Diseases

The invention encompasses intradermal vaccine delivery systems to treat and/or prevent an infectious disease in a subject preferably a human. Infectious diseases that can be treated or prevented by the methods of the present invention are caused by infectious agents including, but not limited to, viruses, bacteria, fungi protozoa, helminths, and parasites.

Examples of viruses that have been found in humans and can be treated by the vaccine delivery systems of the invention include, but are not limited to, Retroviridae (e.g., human immunodeficiency viruses, such as HIV-1 (also referred to as HTLV-III, LAV or HTLV-III/LAV, or HIV-III; and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (e.g., hemorrhagic fever viruses); Reoviridae (e.g., reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted, e.g., Hepatitis C); Norwalk and related viruses, and astroviruses.

Retroviruses that results in infectious diseases in animals and humans and can be treated and/or prevented using the delivery systems and methods of the invention include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Bacterial infections or diseases that can be treated or prevented by the methods of the present invention are caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include but are not limited to infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species. Specific examples of infectious bacteria include but are not limited to: Helicobacter pyloris, Borelia burgdorferi, Legionella pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Fungal diseases that can be treated or prevented by the methods of the present invention include but not limited to aspergilliosis, crytococcosis, sporotrichosis, coccidioidomycosis, paracoccidioidomycosis, histoplasmosis, blastomycosis, zygomycosis, and candidiasis.

Parasitic diseases that can be treated or prevented by the methods of the present invention including, but not limited to, amebiasis, malaria, leishmania, coccidia, giardiasis, cryptosporidiosis, toxoplasmosis, and trypanosomiasis. Also encompassed are infections by various worms, such as but not limited to ascariasis, ancylostomiasis, trichuriasis, strongyloidiasis, toxoccariasis, trichinosis, onchocerciasis, filaria, and dirofilariasis. Also encompassed are infections by various flukes, such as but not limited to schistosomiasis, paragonimiasis, and clonorchiasis. Parasites that cause these diseases can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

The invention also encompasses vaccine compositions to treat and/or prevent cancers, including, but not limited to, neoplasms, tumors, metastases, or any disease or disorder characterized by uncontrolled cell growth. For example, but not by way of limitation, cancers and tumors associated with the cancer and tumor antigens listed supra in Section 5.1.2 may be treated and/or prevented using the vaccine compositions of the invention.

5.6 Screening Methods to Identify Excipients

The invention further encompasses methods of identifying a compound that enhances immunogenicity of an immunogenic or antigenic agent when delivered to the intradermal compartment of a subject's skin. In some embodiments, methods of identifying a compound that enhances immunogenicity of an immunogenic or antigenic agent when delivered to the intradermal compartment of a subject's skin comprise stability measurements of such compounds. In a specific embodiment, candidate compounds or agents are combined with an immunogenic or antigenic agent at a variety of ratios to prepare an immunogenic composition and the resulting composition is monitored for signs of instability relative to the immunogenic or antigenic agent alone in real time and accelerated studies. Stability of the compositions may be assessed using methods known to one skilled in the art and disclosed herein.

In other embodiments methods of identifying a compound that enhances immunogenicity of an immunogenic or antigenic agent when delivered to the intradermal compartment of a subject's skin comprises delivering the candidate compound to the intradermal compartment of a subject's skin. In some embodiments, the candidate compounds are delivered at a variety of concentrations in the intradermal compartment, and monitored for any indications of toxicity using standard methods known to one skilled in the art. Concentrations of candidate compound that do not contribute to degradation and/or toxicity of the immunogenic or antigenic agent in animal pre-trials are then combined with the immunogenic or antigenic and evaluated for adjuvant properties in the intradermal compartment of a subject's skin using methods disclosed and exemplified herein. Adjuvant properties may be assayed using any of the humoral or cell-based assays disclosed in Section 5.4 or any other method known to one skilled in the art.

In other embodiments, in order to identify such compounds an immunogenic or antigenic agent is administered together with a candidate compound into the intradermal compartment of a subject's skin; the immune response resulting from the administration is determined; the same immunogenic or antigenic agent is administered without the candidate compound into intradermal compartment of a second subject, preferably of the same species; the immune response resulting from the second administration is determined using methods known to one skilled in the art; and the immune responses from the first and second administrations are compared. If the immune response from the second administration is greater than the first administration, the compound is characterized as a lead compound, wherein it has adjuvant activity.

The immune response in the subject resulting from the administration of an immunogenic or antigenic agent, with or without the candidate compound, may be determined using any methods known in the art or the methods disclosed herein. The assay for determining the immune response may be in vitro based assays or in vivo based assays, including animal based assays. The invention encompasses measuring humoral based and cell based immune responses using standard methods known to one skilled in the art and described above in Section 5.4. Preferably, the screening assays of the invention are done in a high through put manner.

In a specific embodiment, a method for identifying a compound that enhances immunogenicity of an immunogenic or antigenic agent comprises: (a) delivering an immunogenic composition into an intradermal compartment of a first subject's skin, wherein the immunogenic composition comprises the immunogenic or antigenic agent and the compound; (b) measuring antibody response in a sample obtained from the first subject's serum; (c) delivering and immunogenic composition into an intradermal compartment of a second subject's skin, wherein the immunogenic composition comprises the immunogenic or antigenic agent without the compound, and wherein the first and the second subjects are same species; (d) measuring antibody response in a sample obtained from the second subject's serum; and (e) determining whether the response obtained from the first subject is greater than the response obtained from the second subject. If the response in the sample obtained from the first subject is greater than the second subject, characterizing the compound as an excipient that may be used in the compositions of the invention. Compounds identified by the screening methods of the invention can be used to elicit an enhanced immune response to an antigenic or immunogenic agent when co-administered with the antigenic or immunogenic agent into an intradermal compartment of the subject's skin. Specifically, these compounds can be used in vaccine compositions.

The compounds used in the assays described herein may be members of a library of compounds. In a specific embodiment, the compound is selected from a combinatorial library of compounds. In specific embodiment, the compound is selected from a combinatorial library of organic polymers comprised of nucleic acid, lipid, saccharides where specific non-limiting examples would be peptides of hybrid molecules such as glycoproteins. The invention also encompasses non-organic libraries and methods like those found in WO 01/07642 (the contents of which is incorporated herein by reference in its entirety) can be used to manage the large numbers of candidate compounds. In certain embodiments, the compounds are screened in pools. Once a positive pool has been identified, the individual compounds of that pool are tested separately. In certain embodiments, the pool size is at least 2, at least 5, at least 10, at least 25, at least 50, at least 75, at least 100, at least 150, at least 200, at least 250, or at least 500 compounds.

5.7 Kits

The invention further comprises kits comprising an intradermal administration device and an immunogenic composition of the invention as described herein. In some embodiments, the invention also provides a pharmaceutical pack or kit comprising an immunogenic composition of the invention. In a specific embodiment the invention provides a kit comprising, one or more containers filled with one or more of the components of the immunogenic compositions of the invention, e.g., an antigenic or immunogenic agent, an excipient. In yet another embodiment the pre-filled container further comprises an intradermal delivery device. In another specific embodiment, the kit comprises two containers, one containing an antigenic or immunogenic agent, and the other containing the excipient. Associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLES

Aspects of this invention are illustrated by the following non-limiting examples.

6.1 Immune Response from the Administration of Fluzone™

6.1.1 Preparation of Fluzone™ Inoculum

Prior to preparation of various formulations, the pH of all excipient stock solutions were checked for a neutral pH, i.e., 7.0-7.4. The pH of the solutions was adjusted to neutral as necessary using dilute HCl or NaOH. All excipient stock solutions were sterile filtered through a 0.2 micron Gelman Acrodisc PF syringe filter #4187.

For murine studies, inoculums were prepared by adding 175 μL of Aventis Fluzone™ YR 02/03 and the excipients at a final concentration as denoted in Table 1. Hanks Buffered Saline Solution (HBSS) was used to bring the final volume to 700 μL. A control inoculum was prepared by adding HBSS to 175 μL of Fluzone™ to yield a final volume of 700 μL. Each animal was inoculated by using 100 μL of the prepared inoculums. For non-immune control, A pre-bleed was taken from the animal before immunization. Where each mouse received 25 ul of commercial vaccine per inoculum volume, G. Pigs received 50 ul and rats received 10 ul and 100 ul volumes of commercial vaccine per total inoculum volume.

TABLE 1
Some CONCENTRATIONS OF EXCIPIENTS USED IN
FLUZONE INOCULUMS
(IMMUNOGENICTY AND TISSUE COMPATIBILITY TESTING)

| Excipient | Concentration |
|---|---|
| Amiprilose | 0.3% w/v |
| Amphotericin B | 20, 60, and 180 ng/mL, or 0.000002, |
| | 0.000006 and 0.000018% w/v |
| Bactopeptone | 0.1, 0.3, 0.9 and 1.5% % w/v |
| D-Sorbitol | 2, 5, 10 and 58% w/v |
| Tween 80 | 0.1, 0.3, 0.9, 5 and 10% v/v |
| Sodium Bisulfite | 0.3, 0.9 and 2.7% w/v |
| Triton X-100 | 0.0001, 0.0003 and 0.0009% w/v |
| Triton N-101 | 0.13 and 1.3% w/v |
| Urea | 0.2, 1, 5 and 20% w/v |
| Gelatin | 0.225% and 0.45% w/v |
| DOC | 0.1, 0.5, 1.0 and 5.0% w/v |
| Methylcellulose | 0.06 and 0.18% w/v |
| Lutrol F127 | 5, 10 and 15% w/v |

6.1.2 Administration

Inoculum was injected into Balb/c mice within an hour of preparation. The mice used for inoculation were obtained from Charles River Laboratories and were between 4 and 8 weeks of age. The mice were dry-shaved just prior to injection using a Conair Electric shaver. Approximately 15 minutes prior to the inoculation, each mouse received an intraperitoneal injection of ketamine/xylazine/acepromazine cocktail for sedation. The lower to mid back region was used for injection. Rats were of the Brown Norway Strain and G. Pigs were Hartley Strain. Both were typically 200 grams and larger.

Each murine inoculum was drawn up into a 1 mL latex free syringe (BD Cat. 309628) fixed with a 20 G needle (BD Cat. 305179). After the syringe was loaded the 20 G needle was replaced with a 30 G needle for intradermal (ID) administration. The Mantoux method of ID administration was initially used whereby the skin is tightly pulled and the needle is approached at the most shallow possible angle with the bevel up. The injection volume was pushed in slowly over 5-10 seconds forming the typical “bleb” and then the needle was slowly removed. To prevent the spill over of the inoculum into surrounding tissue space, only one injection was employed and the injection volume per site was kept at 100 μL. Injection volumes were occasionally increased in latter studies. In larger rodent studies, animals received larger overall inoculum volumes to allow for higher percentages of commercial vaccine, however, the volume per site did not exceed 100 ul. In latter murine studies, more efficient ID delivery using 1.0 mm×34 g needles were used and the maximum injection volume per site was 50 ul. Guinea Pig and rat administrations were also performed with the 1.0 mm×34 g needle and the max injection site volume was 50 ul. For all studies, only one immunization was given. A single test bleed was taken twenty-one days later. Animals were monitored for local and systematic indications of toxicity immediately after administration, at 24 hours after the inoculation, and again at three weeks when collecting blood samples. Administration site toxicity was monitored in mice, rats, guinea pigs and swine with deliveries ranging from 1.0 to 3 mm. No signs of local or systematic toxicity were observed in animals.

6.1.3 ELISA Assays

Antibody response to FLUZONE™ was measured by coating an influenza antigen (Influenza APR834, purified/inactivated at 2 mg/mL from Charles River SPAFAS or Alternatively New Calcdonia, Panama or B-Hong Kong lysates from Biodesign Inc.) on a microtiter plate (96-well NUNC IMMUNO-PLATE™ with MAXISORP™ surface). The coating solution was approximately 3.8.mu.g/mL of influenza protein in carbonate buffer (Sigma Chemical Co. Cat. C3041). The coating antigen was exposed to the Nunc plate for one hour at 37.degree. C. The coating solution was discarded and replaced with a blocking solution (phosphate buffered saline with Tween™ 20 (PBS-TW20); Sigma Chemical Co. Cat. P-3563) and 5% w/v nonfat dry milk. The blocking solution was exposed to the plate surface for two hours at 37.degree. C. The blocking solution was subsequently discarded.

Plate surfaces were washed twice with PBS-TW20 and sera from control groups were added. The sera from all animals within a particular group may be assayed individually or pools.

The primary antibody was incubated on the coated and blocked plates for an hour, and afterwards the plates were washed three time with PBS-TW20. A cocktail of anti-mouse horseradish peroxidase conjugate pool, which consisted of Sigma A4416, Southern Biotech 1090-05, Southern Biotech 1070-05, Southern Biotech 1080-05 and Southern Biotech 1100-05, was added. All conjugates were present at a 1:15,000 dilution in the final cocktail. The horseradish peroxidase secondary antibody cocktail was incubated on the plates for an hour at 37° C. The plates were then washed three times with PBS-TW20.

For color development, Sigma T-8665, a TMB substrate, was added, and the color was allowed to develop for 30 minutes in the dark. Color development was stopped by the addition of 0.5 molar sulfuric acid, and the plates were read at 450 nm on a TECAN SUNRISE plate reader.

6.1.4 Results

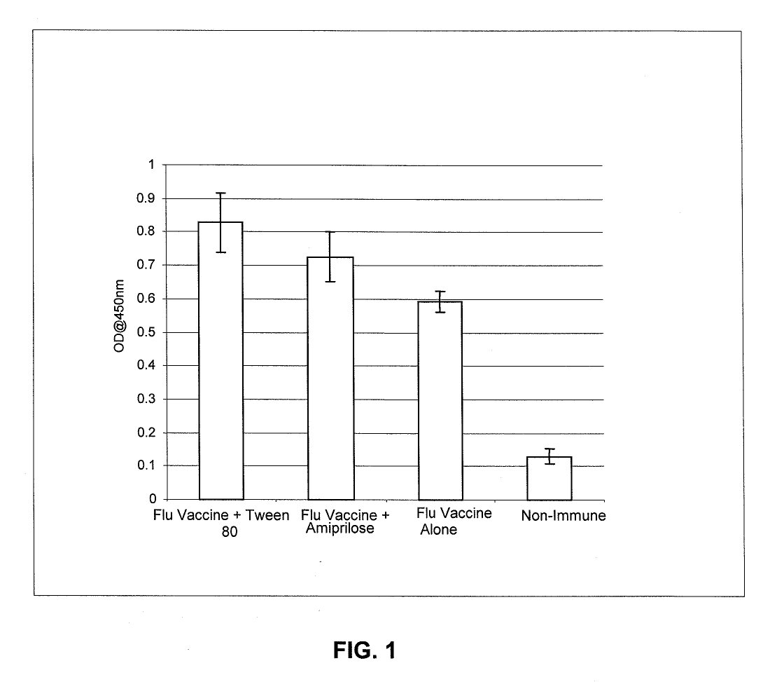

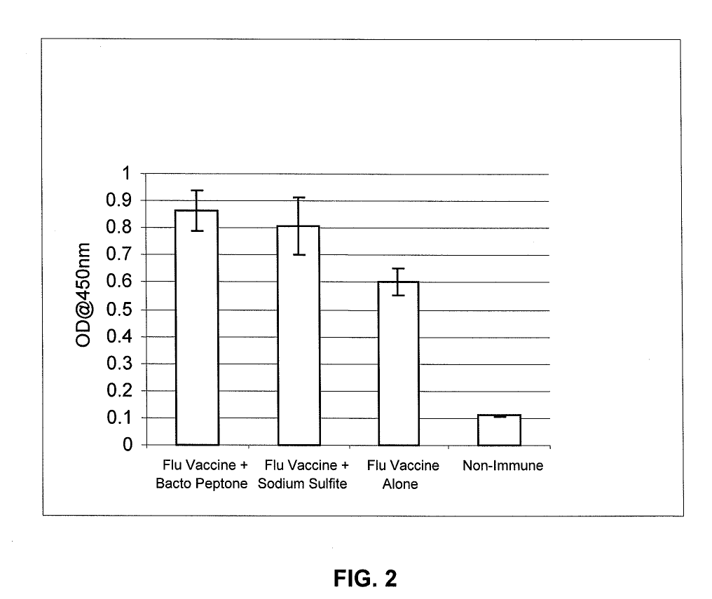

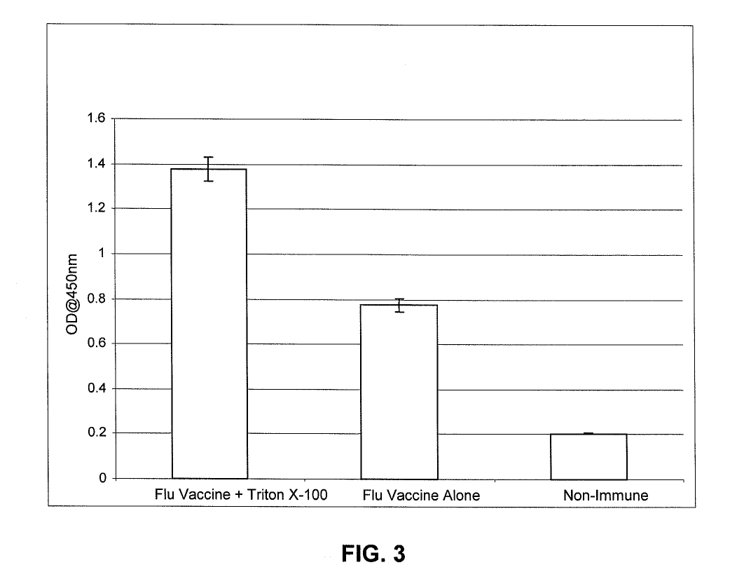

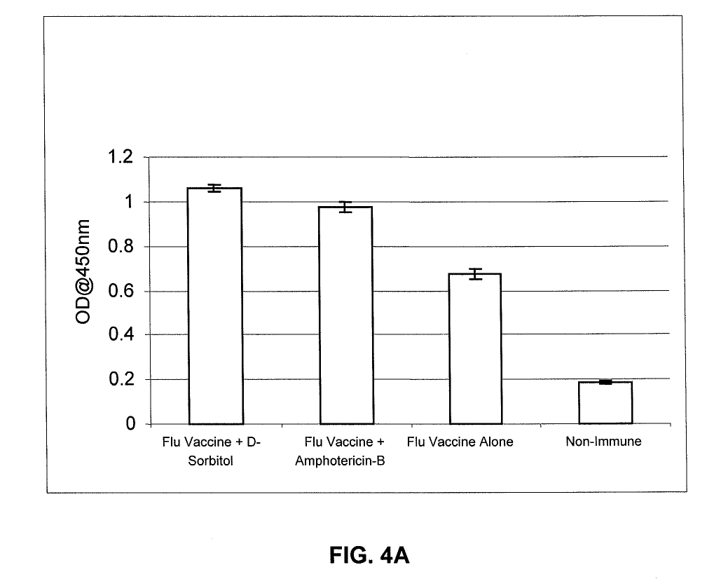

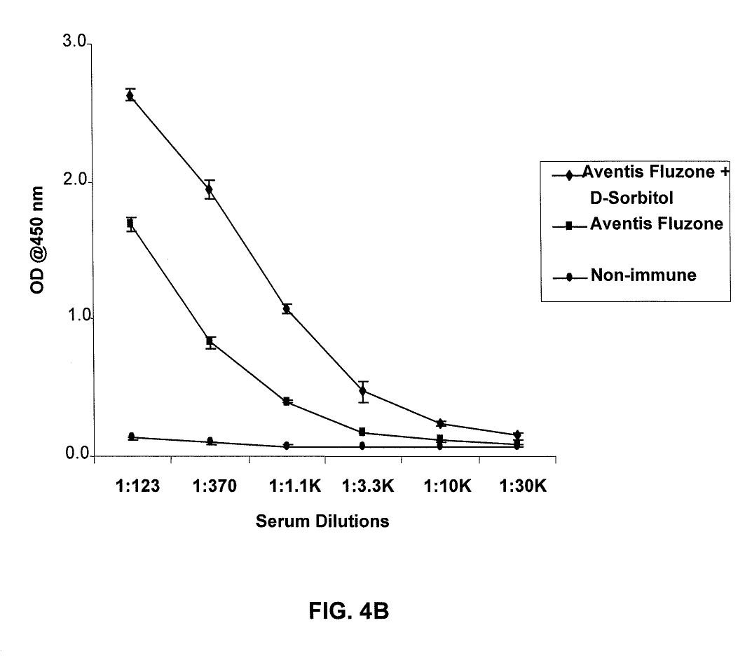

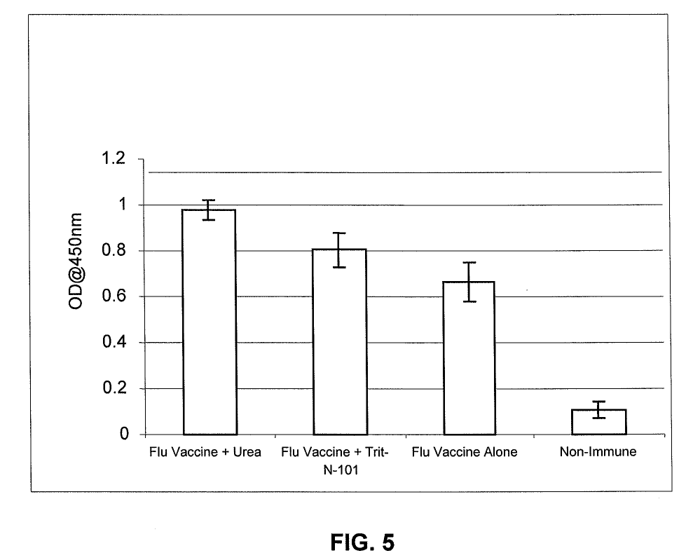

As shown in FIGS. 1-5, 21, 23, 26, 28, 31 and 32, the inoculums that contained any of the excipients listed herein resulted in a greater immune response as compared to the inoculums that contained Fluzone™ alone, or the non-immune control (prebleed). This result clearly shows that these excipients can act as adjuvants when administered together with an antigenic or immunogenic agent into the subject's intradermal compartment.

6.2 Immune Response from the Administration of a Plasmid DNA Comprising a Sequence that Codes Flu Hemagglutinin

6.2.1 Preparation of Inoculum

Prior to preparation of various formulations, the pH of all excipient stock solutions were checked for a neutral pH, i.e., 7.0-7.4. The pH of the solutions was adjusted to neutral as necessary using dilute HCl or NaOH. All excipient stocks were sterile filtered through a 0.2 micron Gelman Acrodisc PF syringe filter #4187.

Inoculums were prepared by adding 350 μg of a plasmid DNA comprising a sequence that encodes flu hemagglutinin (pDNA-HA) and the excipients at a final concentration as denoted in Table 2. HBSS was used to bring the final volume to 700 μl. A control inoculum was prepared by adding HBSS to 350 μg of pDNA-HA to yield a final volume of 700 μL. Each animal was inoculated by using 100 μL of the prepared inoculums. For the non-immune control, a blood sample was taken from animals prior to immunization (prebleed). pDNA immunogen studies were only conducted in Balb/c mice.

TABLE 2
Some CONCENTRATION OF EXCIPIENTS USED IN
INOCULUMS FOR DNA IMMUNOGEN STUDIES

| Excipient | Concentration | |
|---|---|---|
| Apotransferrin | 200 | μg/mL |
| Aprotinin | 20 | μg/mL |
| Bactopeptone | 0.01% | w/v |
| D-sorbitol | 150 | mg/mL |
| Ethanol | 0.2% | v/v |
| Fetuin | 80 ng/100 μL | |
| Gelatin | 0.05% | w/v |
| Glycolic Acid | 0.1, 1.0% | w/v |
| Mannose | 200 | μg/mL |
| Methylcellulose | 0.55% | w/v |
| Sodium Bisulfite | 3 | mg/mL |
| Tri-(n)-butyl | 0.125% | w/v |
| phosphate | | |
| Tween 20 | 0.01% | w/v |
| Urea | 10% | w/v |

6.2.2 Administration

Inoculum was injected into Balb/c mice within an hour of preparation. The mice used for inoculation were obtained from Charles River Laboratories and were between 4 and 8 weeks of age. The mice were dry-shaved just prior to injection using a Conair Electric shaver. Approximately 15 minutes prior to the inoculation, each mouse received an intraperitoneal injection of ketamine/xylazine/acepromazine cocktail for sedation. The lower to mid back region was used for injection.

Each inoculum was drawn up into a 1 mL latex free syringe (BD Cat. 309628) fixed with a 20 G needle (BD Cat. 305179). After the syringe was loaded the 20 G needle was replaced with a 30 G needle for intradermal (ID) administration. The Mantoux method of ID administration was used whereby the skin is tightly pulled and the needle is approached at the most shallow possible angle with the bevel up. The injection volume was pushed in slowly over 5-10 seconds forming the typical “bleb” and then the needle was slowly removed. To prevent the spill over of the inoculum into surrounding tissue space, only one injection was employed and the injection volume was kept at 100 μL.

Animals were monitored for local and systematic indications of toxicity immediately after the first administration (prime), 24 hours after the prime inoculation, 24 hours after the boost and first test bleed that was administered and collected on day 21 respectively. Animals were monitored again at three weeks after the boost, day 42, when the second and final test bleed was taken. No signs of local or systematic toxicity were observed in animals.

6.2.3 ELISA Assay for DNA Immunogen Studies

Antibody response to the various inoculums that comprise pDNA-HA was measured by coating an influenza antigen (Influenza APR834, purified/inactivated at 2 mg/ml from Charles River SPAFAS) on a microtiter plate (96-well NUNC IMMUNO-PLATE™ with MAXISORP™ surface). The coating solution was 3.8.mu.g/mL of influenza protein in carbonate buffer (Sigma Chemical Co. Cat. C3041). The coating antigen was exposed to the Nunc plate for one hour at 37.degree. C. The coating solution was discarded and replaced with a blocking solution (PBS-TW20) and 5% w/v nonfat dry milk. The blocking solution was exposed to the plate surface for two hours at 37.degree. C. The blocking solution was subsequently discarded.

Plate surfaces were washed twice with PBS-TW20 and sera from test/control groups were added. The sera from all mice within a particular group were pooled. The pooled serum was assayed at 1:123 and 1:370 dilutions.

The primary antibody was incubated on the coated and blocked plates for an hour, and afterwards the plates were washed three time with PBS-TW20. A cocktail of anti-mouse horseradish peroxidase conjugate pool, which consisted of Sigma A4416, Southern Biotech 1090-05, Southern Biotech 1070-05, Southern Biotech 1080-05 and Southern Biotech 1100-05, was added. All conjugates were present at a 1:15,000 dilution in the final cocktail. The horseradish peroxidase secondary antibody cocktail was incubated on the plates for an hour at 37° C. The plates were then washed three times with PBS-TW20.

For color development, Sigma T-8665, a TMB substrate, was added, and the color was allowed to develop for 30 minutes in the dark. Color development was stopped by the addition of 0.5 molar sulfuric acid, and the plates were read at 450 nm on a TECAN SUNRISE plate reader.

6.2.4 Results

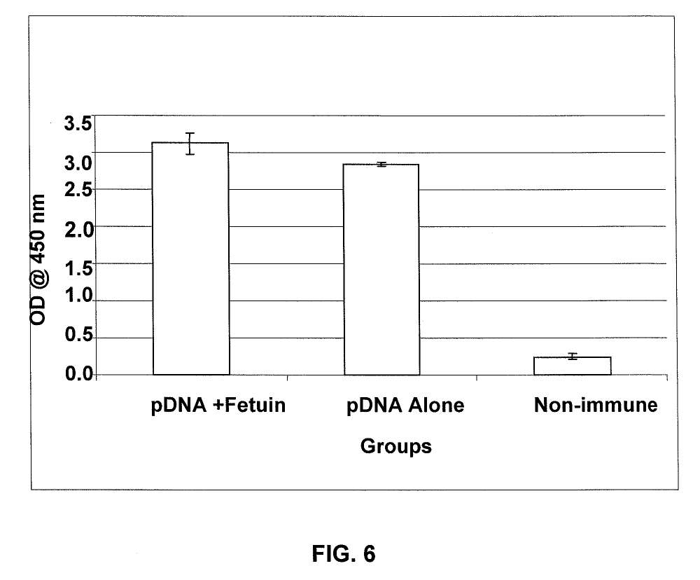

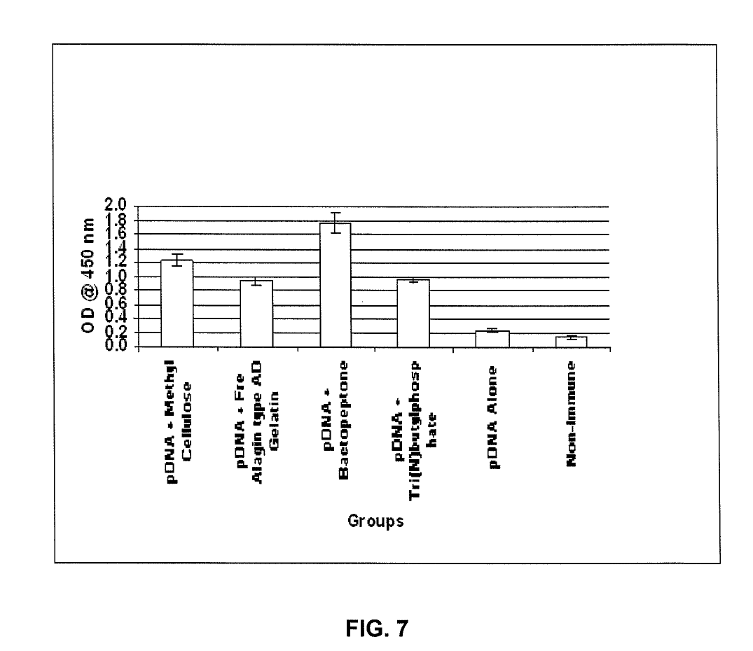

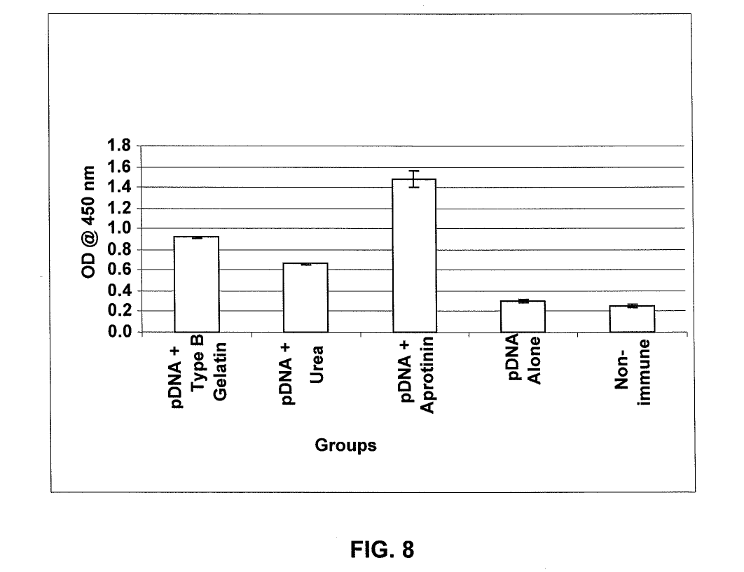

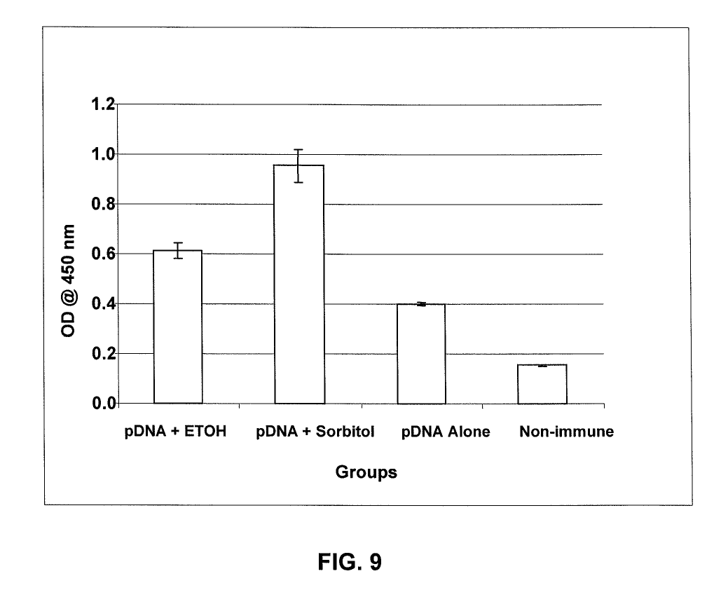

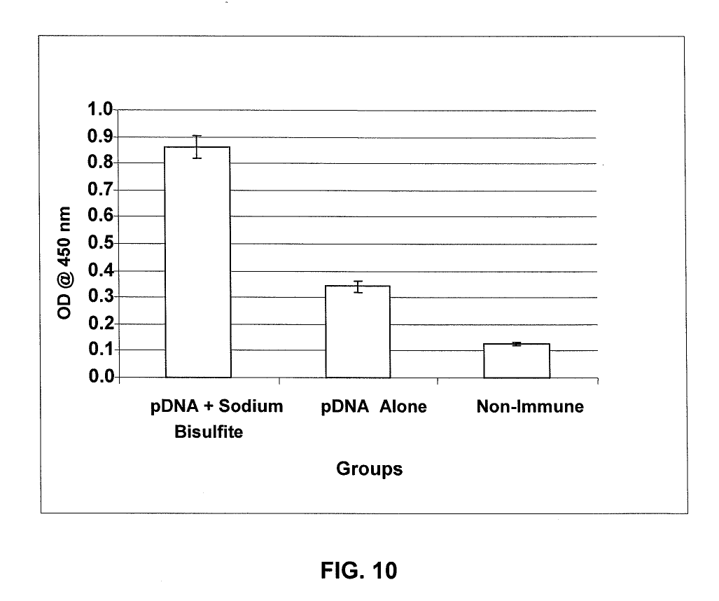

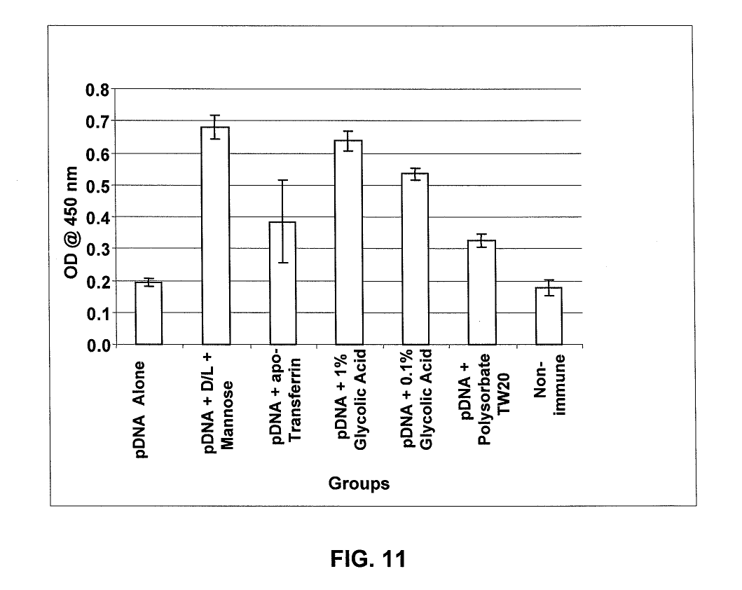

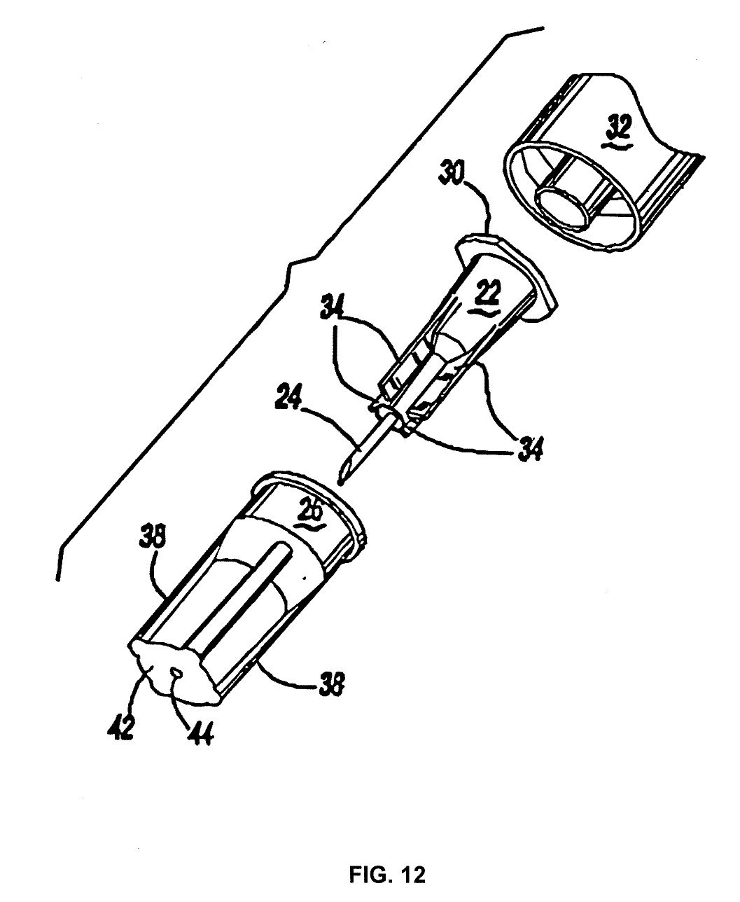

As shown in FIGS. 6-11, all inoculums that contain any of the excipients listed herein-elicited in an increase immune response from the animals as compared to the inoculums that contained pDNA-HA alone, or non-immune control (pre-bleed). These results clearly show that these excipients can act as adjuvants when administered together with an antigenic or immunogenic agent into the intradermal compartment. Some agents were flagged as having adjuvant activity after analyzing the first test bleed and others were flagged after analyzing the second test bleed.

6.3 Initial Range Finding Studies Conducted in Mice

Inoculums that contain FLUZONE™ and various excipients were prepared and intradermally administered into the animals using the methods substantially identical to those described in Sections 6.1.1-2, above. The inoculums were prepared in such a way that each inoculum contains FLUZONE™ and an excipient at a varying amount. The immune responses were measured using the methods substantially identical to those described in Section 6.1.3, above. The results are illustrated in Table 3.

TABLE 3
IMMUNE RESPONSE Vs. EXCIPIENT CONCENTRATION

| | | | Trend in immune response |
|---|---|---|---|
| | | | as indicated by ELISA signal |
| | | | (1:123 serum screening |
| Excipient | Conc. | | dilution) |
| Ethanol | 0.05% | v/v | 0.901 |
| | 0.15% | v/v | 2.742 |
| | 0.45% | v/v | 1.530 |
| Sodium | 0.3% | w/v | 0.808 |
| Bisulfite | 0.9% | w/v | 1.833 |
| | 2.7% | w/v | 2.048 |
| Amphotericin B | 20 | ng/ml | 0.975 |
| | 60 | ng/ml | 1.575 |
| | 180 | ng/ml | 1.018 |
| D-sorbitol | 2% | w/v | 1.062 |
| | 10% | w/v | 1.102 |
| | 58% | w/v | 1.58 |
| Gelatin | 0.05% | w/v | 0.983 |
| | 0.15% | w/v | 1.104 |
| | 0.45% | w/v | 1.183 |
| Bactopeptone | 0.1% | w/v | 0.846 |
| | 0.3% | w/v | 2.647 |
| | 0.9% | w/v | 2.330 |
| Methyl | 0.06% | w/v | 0.844 |
| Cellulose | 0.18% | w/v | 2.757 |
| | — | | — |
| Triton N-101 | 0.13% | w/v | 0.805 |
| | 1.3% | w/v | 2.035 |
| Triton X-100 | 0.0001% | w/v | 1.321 |
| | 0.0009% | w/v | 1.214 |
| Tween 80 | 0.1% | w/v | 0.829 |
| | 0.3% | w/v | 1.599 |
| | 0.9% | w/v | 2.647 |
| Urea | 1% | w/v | 0.979 |
| | 5% | w/v | 1.585 |
| | 20% | w/v | 1.555 |

6.3.1.1 Hemaglutinin Inhibition Assay Used in Mouse, Rat and G. Pig Studies

Preparation of Chicken Red Blood Cells: Chicken Red Blood Cells (cRBC, 5 ml packed) were obtained from Charles River Laboratories (Cat. #S8776). cRBC was equally distribuited into four Flacon® Blue Max™ 50 ml polyethylene conical tubes, and centrifuged at 1500 rpm for 5-7 minutes at 4° C. Shipping buffer was removed from cRBC. Sodium chloride solution (0.9%) was added in 5 ml increments onto the cRBC pellet, and the pellet was resuspended. Combining the resuspended pellets from two of the first-wash, the volume was adjusted to 45 ml with sodium chloride solution (0.9%). The mixture was centrifuged at 1500 rpm for 5-7 minutes at 4° C., and the supernatant was discarded. Again, sodium chloride solution (0.9%) was added in 5 ml increments onto the cRBC pellet, and the pellet was resuspended. The resuspended pellets from two second-wash were combined, and the volume was adjusted to 45 ml with sodium chloride solution (0.9%). The mixture was centrifuged at 1500 rpm for 5-7 minutes at 4° C., and the supernatant discarded. Ten percent cRBC solution was prepared by resuspending the final pellet in ten times the original volume.

Titration of the Influenza antigen Working stock to verify HA content: Prior to performing the HA Inhibition Assay, the HA titer of the viral lysate working stock must be validated. The working stock should be 8HA per 50 μl. Fresh 0.5% cRBC reagent was prepared daily. Predetermined dilution of the viral lysate to yield the presumptive 8 HA working stock was performed. Dilutions were prepared with sodium chloride solution (0.9%).

Sodium chloride solution (0.9%, 50 μl) was distributed into the wells of a Falcon® Non-Tissue Culture Treated Plate, 96 well, U-Bottom with Low Evaporation Lid. The presumptive 8HA/50 μl working stock (100 μl) was distributed into a single row or column of “start wells.” Half volume (50 μl) of the stock was transferred from the start well to a second well, creating a 1:2 dilution. Using the 1:2 dilution, repeat the process and continue until the dilution series was complete. A complete dilution set had wells containing 0.0625 HA to 8HA. cRBC reagent (0.5%, 50 μl) was distributed into each well containing some level of HA, and the assay was allowed to incubate for 45 minutes at room temperature, ensuring that the plate is not jostled.

Interpretation—The cRBC's will settle in the well if too little viral lysate HA is present in the dilution to ensure hemagglutination. Any well containing partial or total settling of the cRBC's to the bottom of the well is negative. The last well with complete suspension of the cRBC's in the solution is the HA titer of the viral lysate stock. If the stock was truly an 8HA per 50 μl stock, then upon retitration, the last positive wells contain 1HA.

Measurement of HA specific Antibody Titer by HAI: Sera were collected and used as test samples. Fresh cRBC reagent was prepared daily. Sodium Chloride solution (0.9%) was added to wells of a Falcon® Non-Tissue Culture Treated Plate, 96 well, U-Bottom with Low Evaporation Lid. Viral lysate stock (8 HA/50 μl) was added to wells. Appropriate volume of test serum was added to a single row or column of “start wells,” and a serial dilution was performed by transferring 50 μl of the serum dilution from the “start wells” into the next well, creating a 1:2 dilution. When completed, wells contained a serial serum dilution and a constant amount of viral lysate antigen, being 4 HA per well. cRBC reagent (0.5%, 50 μl) was added to each well, including negative control wells, which contained no HA. The assay was allowed to incubate for 45 minutes at room temperature, ensuring that the plate is not jostled. For determination, plates were tilted at a 70-degree angle for 5 minutes, and viewed on alight box.

HAI assays were performed with A-New Caledonia (H1N1), A-Panama (H3N2) and B-Hong Kong antigen as single test antigens and trivalent pools.

6.4 Identifying Operating Concentrations and Benefits with Tween™ (Related Compounds)

The objective of these studies was to determine the optimum concentration ranges for delivery of vaccine formulations comprising non-ionic surfactant detergents and related compounds to the intradermal compartment. These studies show non-ionic surfactant excipients function as adjuvants when delivered to the ID compartment in accordance with the methods of the invention. The operating concentrations vary with needle depth (1.00 mm vs. 1.5 mm vs. 2.0 mm vs. 3.00 mm). Each surfactant has a different operating range where adjuvant properties are demonstrated and tissue irritation is avoided or minimized (≦2 Draize Score). Many of the concentration ranges cited in the literature for such agents are for manufacturing purposes. The manufacturing concentrations of such agents are actually toxic and damaging to the tissue when delivered to the ID compartment. In other cases the concentrations are too low to have an adjuvant-like effect. Herin, Tween 80 and other surfactants are shown to enhance seroconversion, mean titer, while avoiding irreversible tissue damage.

6.4.1 Results

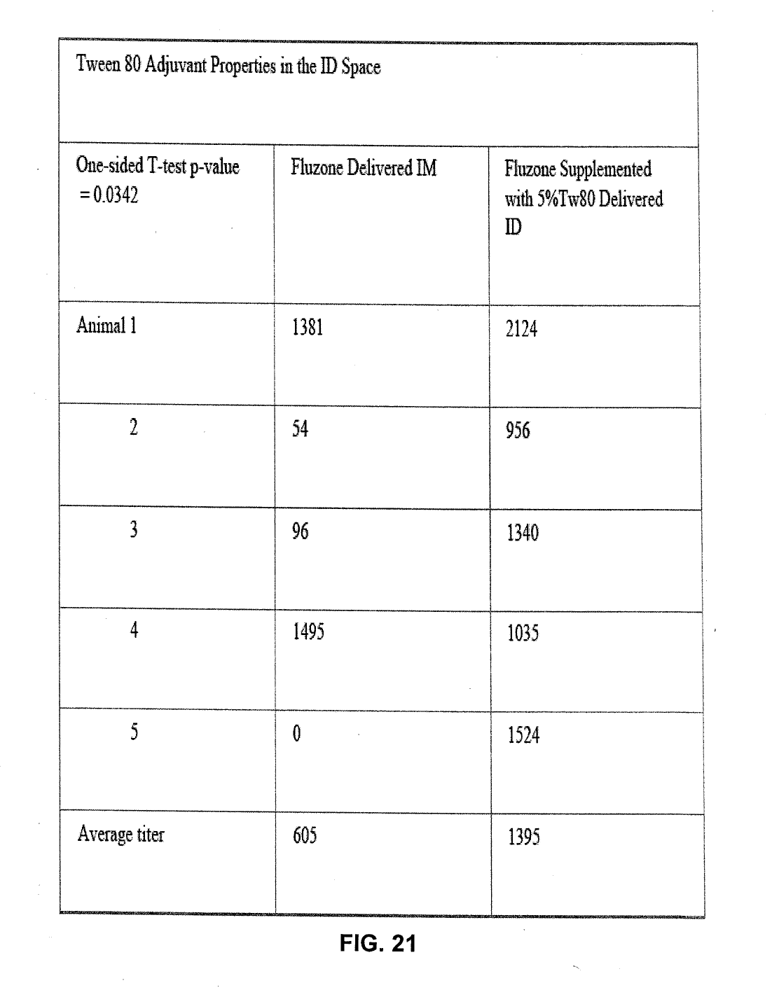

Tween™ 80 enhances Seroconversion: Using the methods already described above, Tween™ 80 at 5% led to 100% seroconversion in FIG. 21. The study used Balb/c mice. The enhancement to seroconversion was observed using an ELISA assay with PR8 test antigen. Animals were given FLUZONE™+Tween™ 80 by the ID route vs. FLUZONE™ given IM unsupplemented. The ID group outperformed the IM group.

Tween™ 80 enhances Mean titer: As shown in FIG. 21, the 5% Tween™ 80 supplemented FLUZONE™ delivered ID led to an average titer of 1:1395, where the unsupplemented FLUZONE™ delivered IM had an average titer of 1:605. A t-test was applied and a p-value of less than 0.05 was assigned, indicating significant change. The ID inoculum was delivered ID-Mantoux using standard syringe and needle. All immune response data discussed were generated in Balb/c mice.

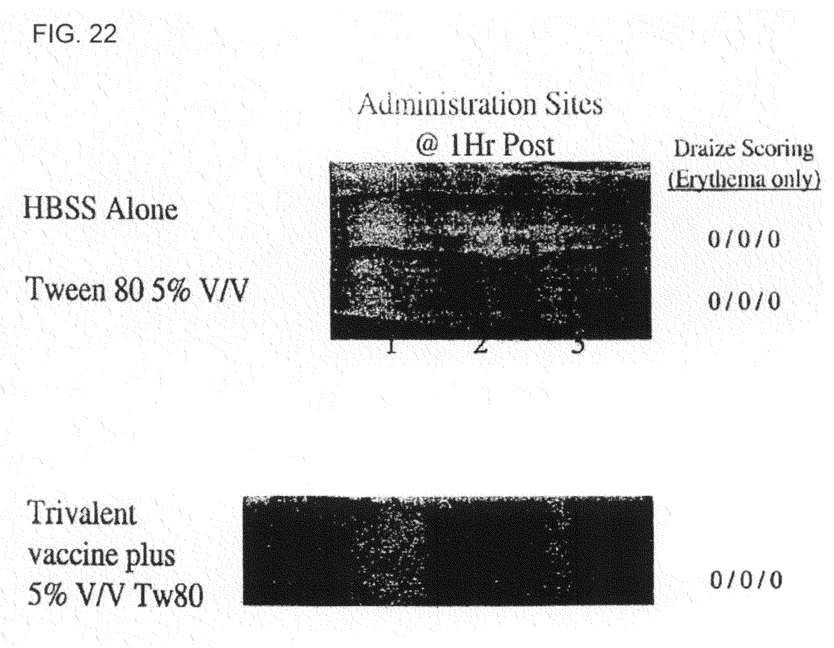

Tween™ 80 Skin Compatibility: Swine skin compatibility studies were performed with micromedica needles of 31 g and 1.5 mm in length.-Tween™ 80 was well tolerated in the ID space, (FIG. 22). In this experiment, swine received 5% V/V Tween™ 80 alone and FLUZONE™ supplemented with 5% V/V Tween 80. All draize scores were acceptable (<2) at 1 hour post administration.

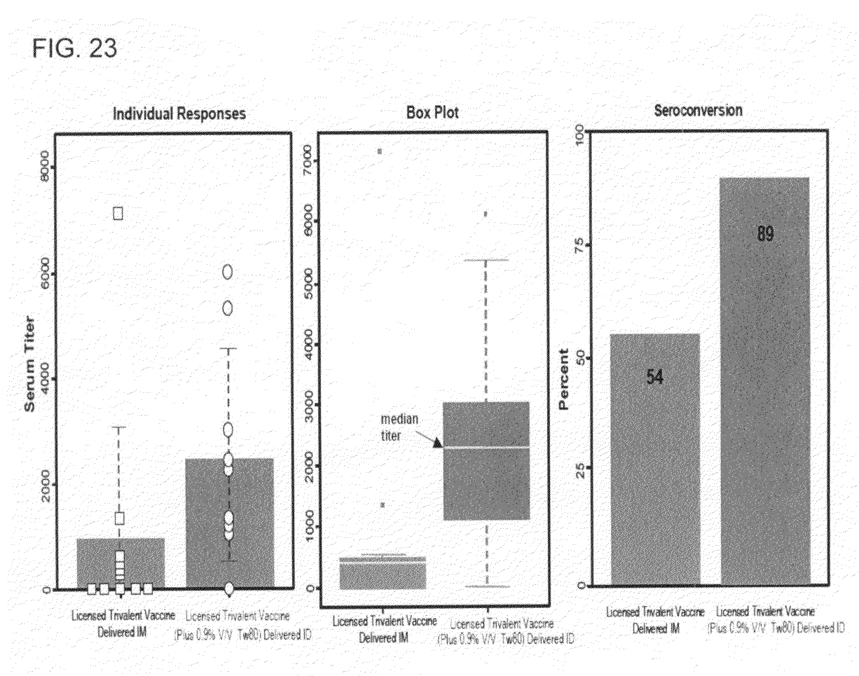

Tween™ 80 Elevating the performance of ID delivers over IM: Tween™ 80 (0.9% V/V) delivered ID with a trivalent vaccine led to higher mean titers, higher median titers and higher seroconversion as compared to the commercial trivalent vaccine delivered IM (FIG. 23). While 0.9% V/V Tw80 performs reasonably well in regards to the immuno enhancement, this concentration may occasionally fail to perform. The micromedica needle used to generate the ID data in FIG. 23 was a 34 g.times.1 mm needle. A murine model was used.

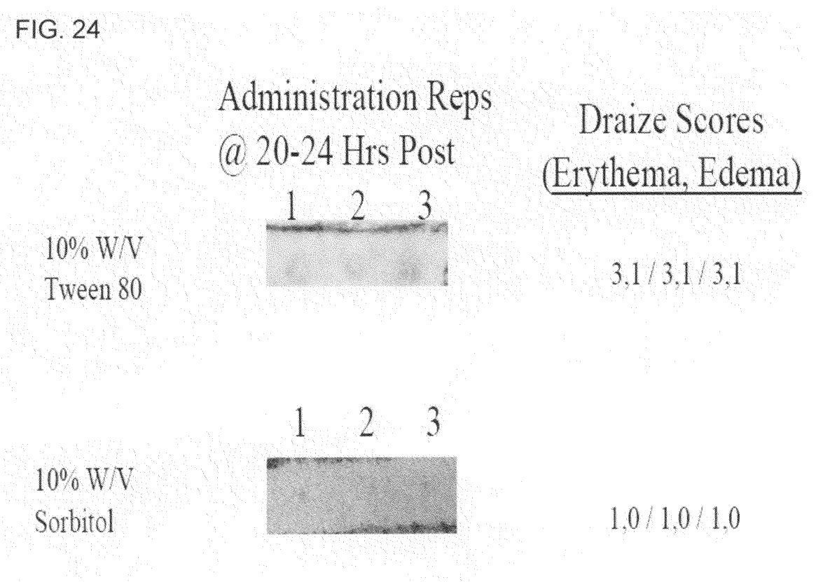

Tween™ 80 Operating Concentrations vs. other surfactants: Sorbitol and sorbitiol derivatives such as Tween 80 have different skin compatibility profiles and particularly so in the ID space. Therefore the functional range for each agent must be determined separately. For example as illustrated in this study: although Tween™ 80 was not well tolerated at 10% W/V when delivered to the 1-2 mm depth; 10% W/V sorbitol was well tolerated (FIG. 24). At 20-24 hours post administration the 10% W/V Tween™ 80 had moderate to severe erythema spanning the initial bleb where the 10% W/V sorbitol caused only mild erythema at the needle penetration site. The study was conducted in Yorkshire swine.

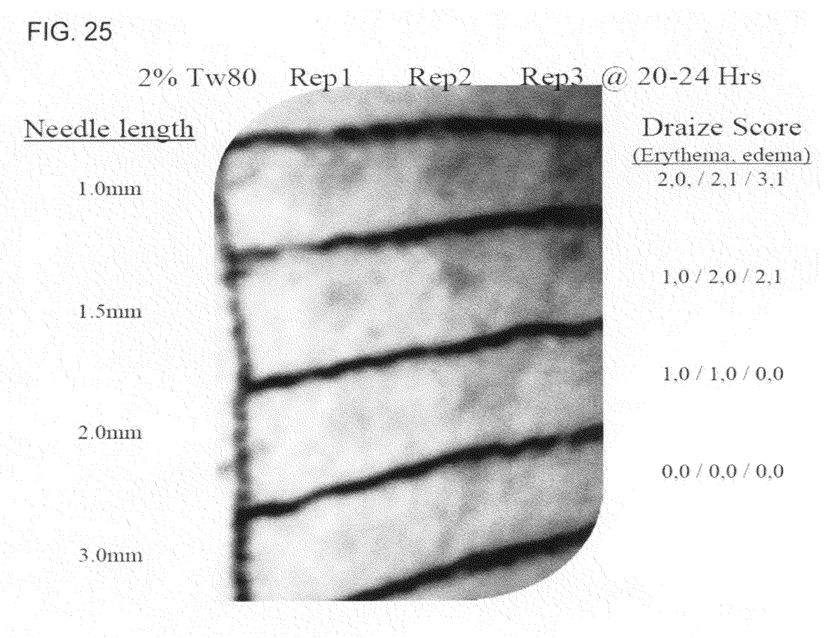

Tween™ 80 operating range varies with tissue depth: In this experiment, Tween™ 80 showed different skin compatibility profiles, varying with needle depth. Specifically, the Yorkshire swine data at 20-24 hours post administration showed how a 2% Tween™ 80 solution was tolerated when delivered with 11.0 mm, 1.5 mm, 2.0 mm and 3.0 mm needle (FIG. 25). At approximately 20-24 hours after the administration, a 3.0 mm delivery yielded good skin results, with a draize score of 0.0. Skin reactions for a particular concentration of Tween™ 80 improved with depth. These studies showed that as the injections become shallower the level of visible irritation increases.

Tween™ 80 Preferred concentrations avoid hemolysis: Surfactants/detergents can lyse RBCs. Blood from Yorkshire swine that received 6.times.200 ul doses containing 5.0% Tween™ 80 were collected. No hemolysis was observed was in blood taken immediately from the systemic circulation. A 31 g.times. 1.5 mm needle was used in the study.

Tween™ 80 enhances dose sparing features of delivery, enhances seroprotection and seroconversion: The addition of the excipient Tween™ 80 to a commercial vaccine formulation provides adjuvant-like properties when delivered ID by Microneedles.

Female Brown Norway (BN) rats (n=10/group) were immunized by ID delivery using Microneedles (34 gauge needle inserted into a 3.5″ long catheter to an exposed needle length of 1.0 mm). ID delivery by Microneedles consisted of 2 bolus injections of 100 μl by hand to either side of the lower back of the rat, approximately 10 seconds in duration (each bolus), using an attached 1 cc syringe. Rats were immunized with either of two different doses of the 2003/2004 season of FLUZONE™ (Aventis Pasteur, Swiftwater, Pa.), for a total of 9.mu.g (high dose) or 0.9.mu.g (low dose) hemagglutinin (HA) per rat, 3.mu.g or 0.3.mu.g HA of each strain of influenza in the vaccine (A/New Calcdonia/20/99 H1N1, A/Panama/2007/99H3N2, and B/Hong Kong/1434/2002).

The rats were bled 21 days after immunization and their serum assayed for neutralizing antibody titres using the hemagglutination inhibition (HAI) assay, and for influenza-specific antibody titres by ELISA. Both assays were performed against the H3N2 strain of influenza in the FLUZONE™ formulation to characterize the immune response.

TABLE 4
Immune response in Brown Norway rats, assayed against
Influenza A/Panama/2007/99 (H3N2) following immunization
with Low and High doses of Fluzone by ID delivery by
Microneedle.

| | Fluzone | | Fluzone + Tween 80 | |
|---|---|---|---|---|
| | ID_{Low} | ID_{High} | ID_{Low} | ID_{High} |
| HAI Titre | 23 ± 4.2 | 58 ± 7.6 | 102 ± 17 | 304 ± 64 |
| ELISA Titre | 3200 ± 413 | 22400 ± 3963 | 48000 ± 10506 | 94720 ± 15289 |
| % Seroprotection¹ | 30% | 90% | 90% | 100% |
| % Seroconversion² | 90% | 100% | 100% | 100% |

¹HAI Titre ≧ 40
²HAI Titre ≧ 10

The addition of the excipient Tween™ 80 to FLUZONE™ provides an adjuvant-like effect when delivered ID by Microneedles. When assayed against the H3N2 strain, the addition of 5% Tween80 to both a low or high dose of FLUZONE™ administered ID increased the mean HAI titre 5-fold and the mean ELISA titre up to 15-fold relative to that achieved from ID delivery of FLUZONE™ alone (Table 4). Also, the addition of Tween™ 80 increased the seroprotection rate from 30 to 90% for the ID low dose groups, and from 90 to 100% for the ID high dose groups. Similarly, the seroconversion rate rose from 90 to 100% for the low dose ID groups and remained unchanged at 100% for the high dose ID groups (Table 4). This combination of ID delivery and Tween™ 80 may be of particular benefit to human populations that do not typically respond strongly to influenza vaccine; e.g., the elderly, infants and the immunocompromised.

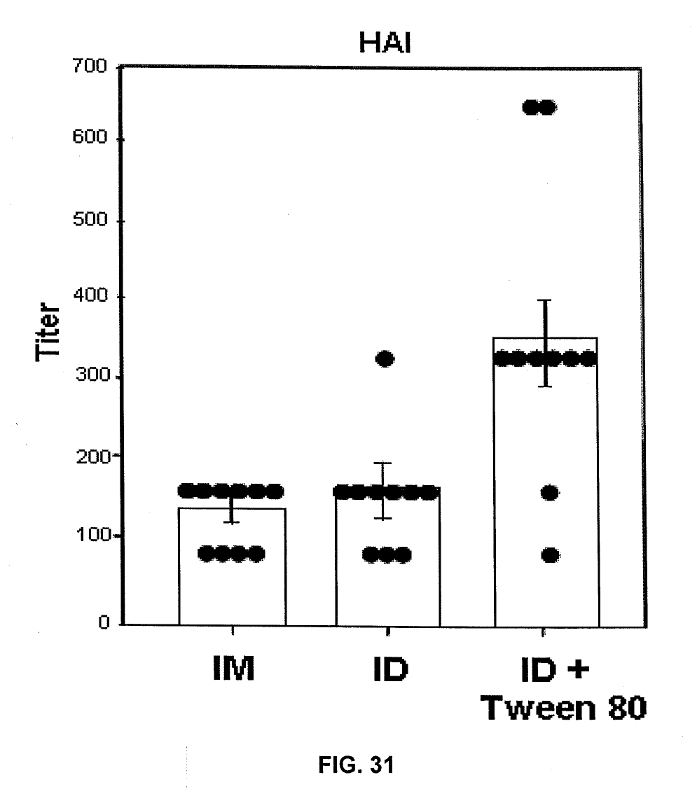

Tween™ 80 enhanced hemagglutin specific titer to trivalent test antigen. In a study using Hartley Guinea Pigs, a FLUZONE™ inoculum supplemented with 5.0% V/V Tween™ 80 was delivered ID. The supplemented intradermal formulation outperformed FLUZONE™ (alone) delivered ID and FLUZONE™ (alone) delivered IM. Serum samples were assayed by the HAI method described earlier. The trivalent test antigen was comprised of New Calcdonia, Panama and B-Hong Kong antigen. The trivalent test antigen was constructed with equal parts of HA. Results represented in FIG. 31. A 34 g.times.1.0 mm needle was used in this study.

Tween™ 80 matches the preferred excipient profile. FIG. 35 illustrates an excipient selected for the intradermal tissue according to the instant invention. Tween™ 80 has a profile similar to the “Excipient-A” in the illustration having a slope greater than 0.125. Whereby a 5% v/v soln of Tween™ 80 is at the maximum operating concentration at 1.0 mm depth, and can be used successfully at 10% v/v at the 3 mm depth.

Deoxycholate

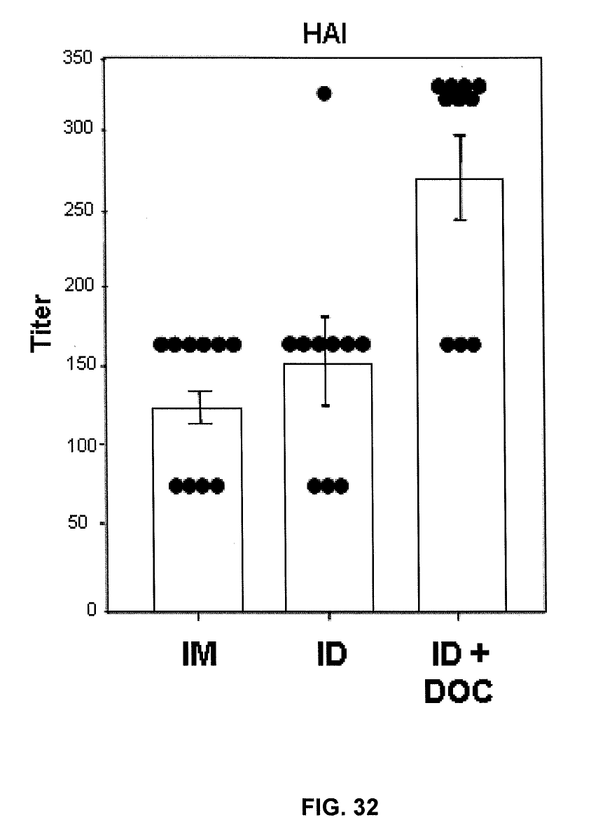

DOC enhanced hemaglutin specific titer to trivalent test antigen: In a study using Hartley Guinea Pigs, a FLUZONE™ inoculum was supplemented with 0.1% w/v sodium deoxycholate and delivered ID. The supplemented ID formulation outperformed FLUZONE™ (alone) delivered ID and FLUZONE™ (alone) delivered IM. Serum samples were assayed by the HAI method described earlier. The trivalent test antigen was comprised of New Calcdonia, Panama and B-Hong Kong antigen. The trivalent test antigen was constructed with equal parts of HA. Results represented in FIG. 32. A 34 g.times. 1.0 mm needle was used in this study.

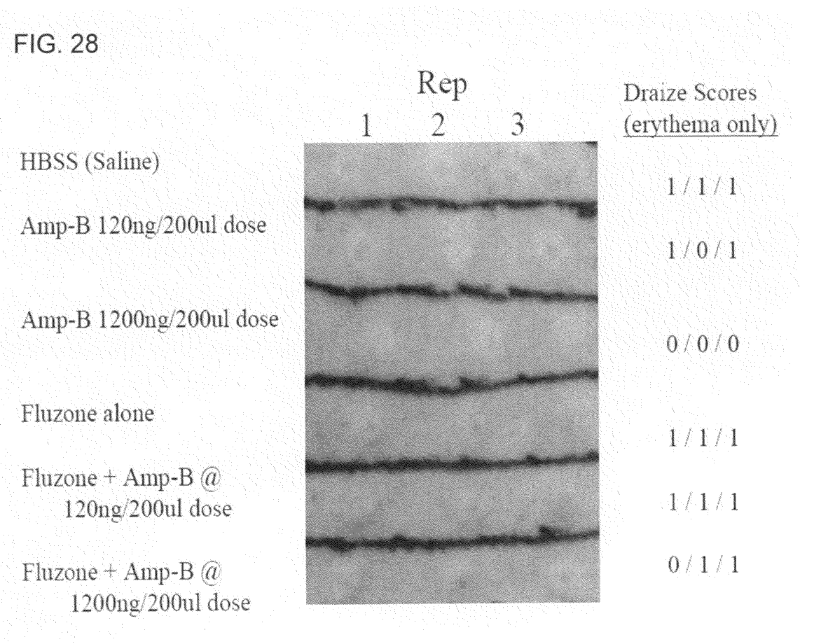

DOC enhancing seroconversion: When deoxycholate, a virus splitting agent, was delivered to the ID space it demonstrated immunopotentiating characteristics as seen in FIG. 28. Here a trivalent vaccine, FLUZONE™, was delivered IM without DOC and only 1 in 5 animals seroconverted 21 days after immunization. The same graph shows however that 5 of 5 animals receiving DOC-supplemented FLUZONE™ by the ID route were seroconverted. The ID formulation containing 0.1% sodium deoxycholate delivered the best median titer. The study was conducted in Balb/c mice.

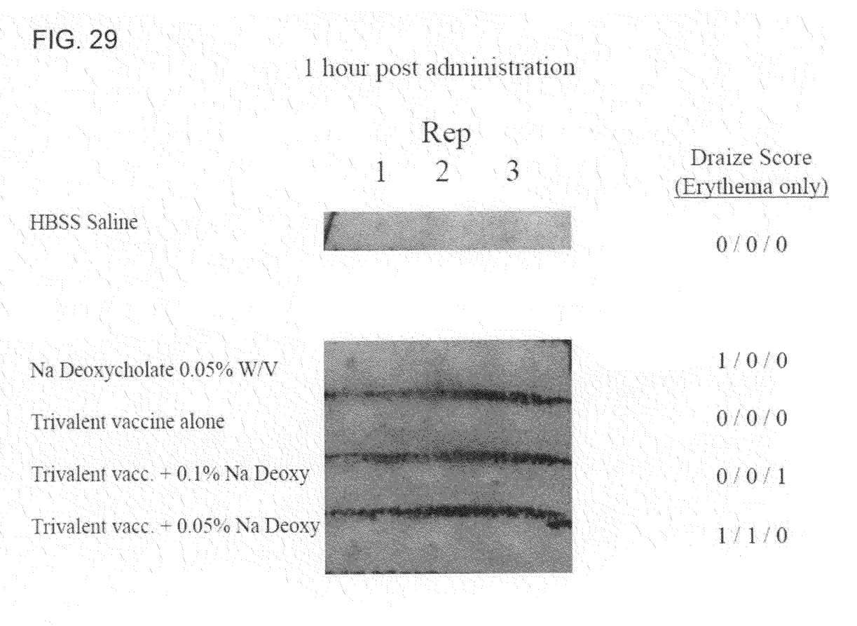

DOC operating ranges vary with tissue depths: Inoculums containing trivalent vaccine and varying concentrations of deoxycholate were evaluated for skin compatibility in Yorkshire swine. Inoculums containing 0.05 and 0.1%+/−trivalent vaccine performed well at the 1.5 mm depth (FIG. 29). In previous studies (data not shown) concentrations of deoxycholate at 0.5% W/V and higher could not be tolerated at the 1.5 mm depth. At this point the preferred range for a 1.5 mm delivery is expected to be 0.07 to 0.15% W/V, with the next best range expanding from 0.01 to 0.3% w/v. As described for the Tweens™, the upper concentration can increase with deeper injections. For example, a 3 mm administration may tolerate up to 0.6% w/v deoxycholate or higher.

Identifying Operating Concentrations and Benefits for Other Excipients

The objective of these studies was to determine the optimum parameters including concentration ranges for delivery of vaccine formulations comprising excipients which are traditionally used in manufacturing processes such as stabilizers and preservatives, with examples being gelatin and amphotericin-B, bacto peptone (a component of culture media) and tri-butyl phosphate (a diluent used with the splitting agent). Sometimes residual amounts of these agents can carry over into the final vaccine formula and can have unexpected properties.

6.4.2 Gelatin:

Gelatin formulations with adjuvant properties and good flow characteristics: A preferred range for gelatin was determined to be 0.01 to 0.225 W/V. Higher concentrations of gelatin forms solids at room temperature and particularly at refrigeration temperature. These observations were made while working with a national formulary grade of gelatin (porcine origin). A 0.225% w/v gelatin passes easily through a 34 gauge needle and is well tolerated at 1-3 mm tissue depths.

Gelatin Enhances Seroconversion and Median

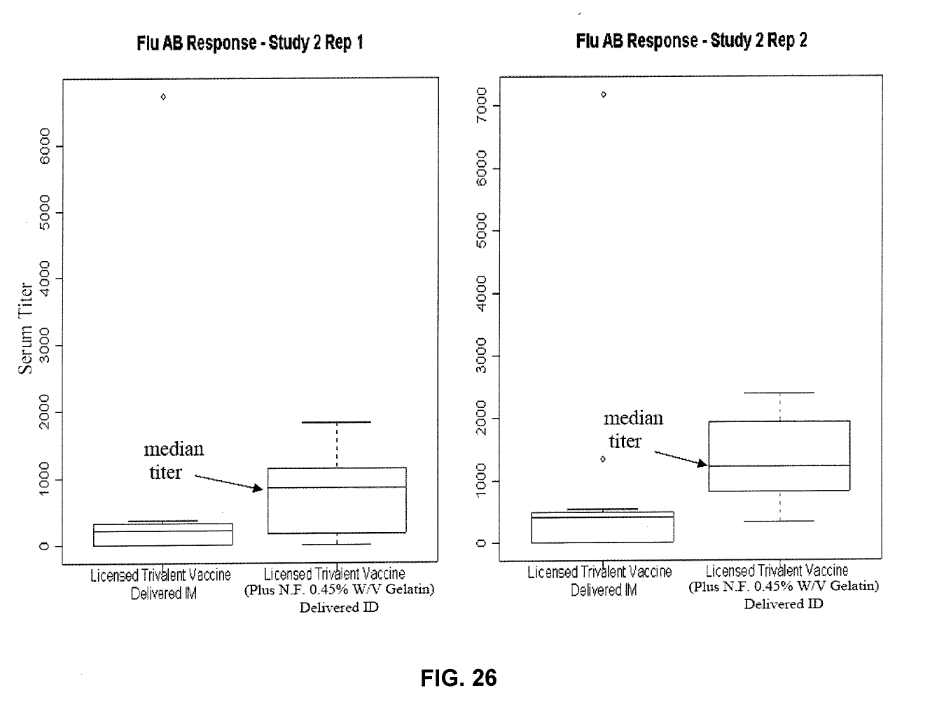

Gelatin used at 0.45 W/V is capable of enhancing immunogenicity of target antigen. FIG. 26 shows an intradermal formulation with gelatin outperforming an intramuscular formulation. A Fluzone trivalent formula supplemented with gelatin was delivered ID and straight Fluzone was delivered IM. An ELISA assay was used and the test antigen was PR8. The animal model was Balb/c and needle was 34 g×1 mm.

6.4.3 Amphotericin-B:

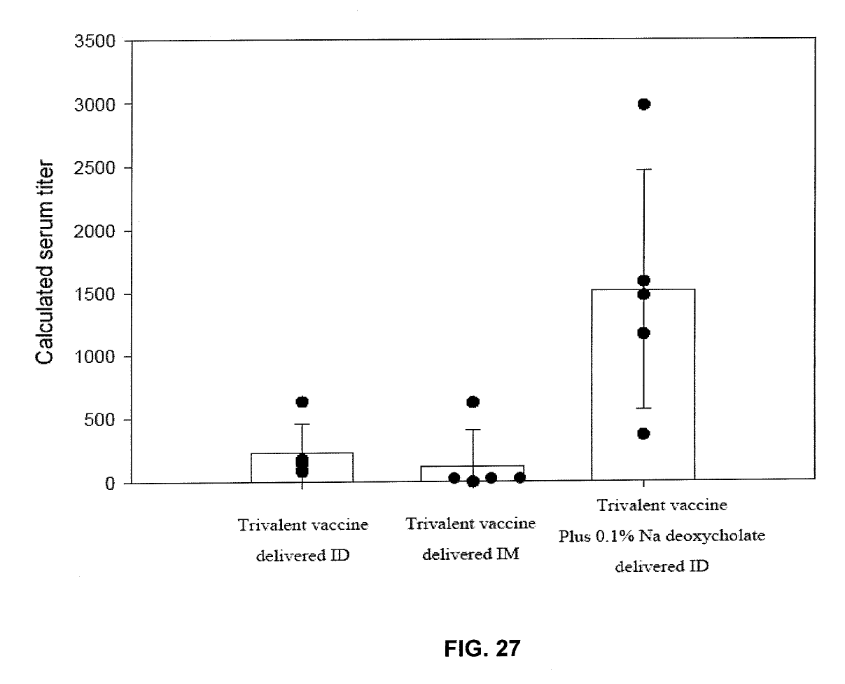

Amp-B Skin Compatibility In Yorkshire Swine studies, animals tolerated 600 ng/100 ul or 1200 ng/200 ul total dose. As evident by the Draize score analysis (FIG. 27), Amp-B was well tolerated when evaluated alone and as a mixture with FLUZONE® vaccine. Analysis was performed at the 1.5 mm depth.

6.4.4 Bactopeptone

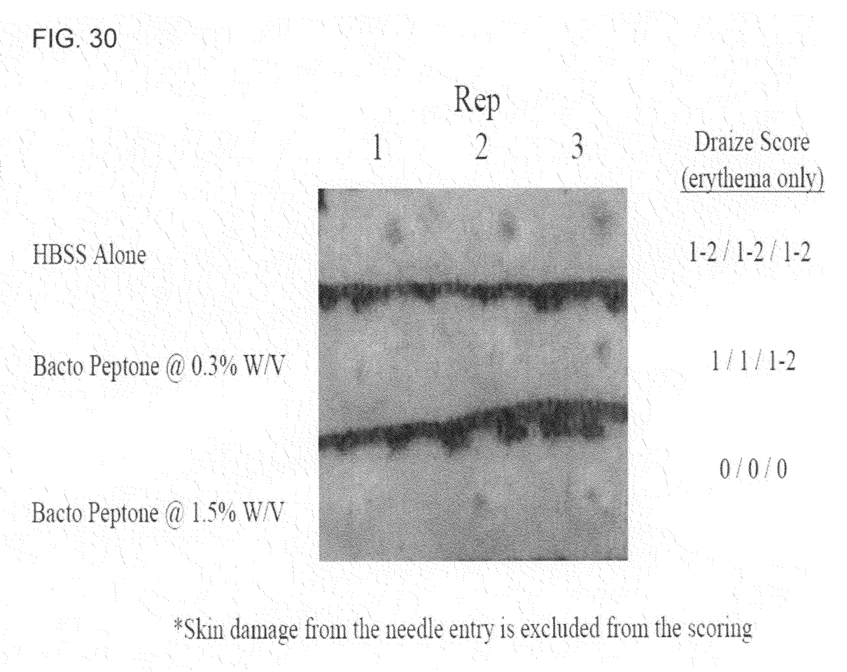

Peptone reduces visible irritation. Another unexpected result was an excipient that calms the irritation caused by the vaccine itself and diluent. The bactopeptone excipient has been shown to mask the irritation often seen at the site of administration. As shown in FIG. 30, Hanks Buffered Saline (diluent) alone will sometimes cause mild irritation. The bactopeptone, excipient, when added has a calming affect, reducing the draize score. The positive attribute was particularly evident when bactopeptone was used at 1.5% w/v. The experiment was conducted in Yorkshire swine and the tissue depth was 1.5 mm.

6.5 Draize Scoring of Various Excipients

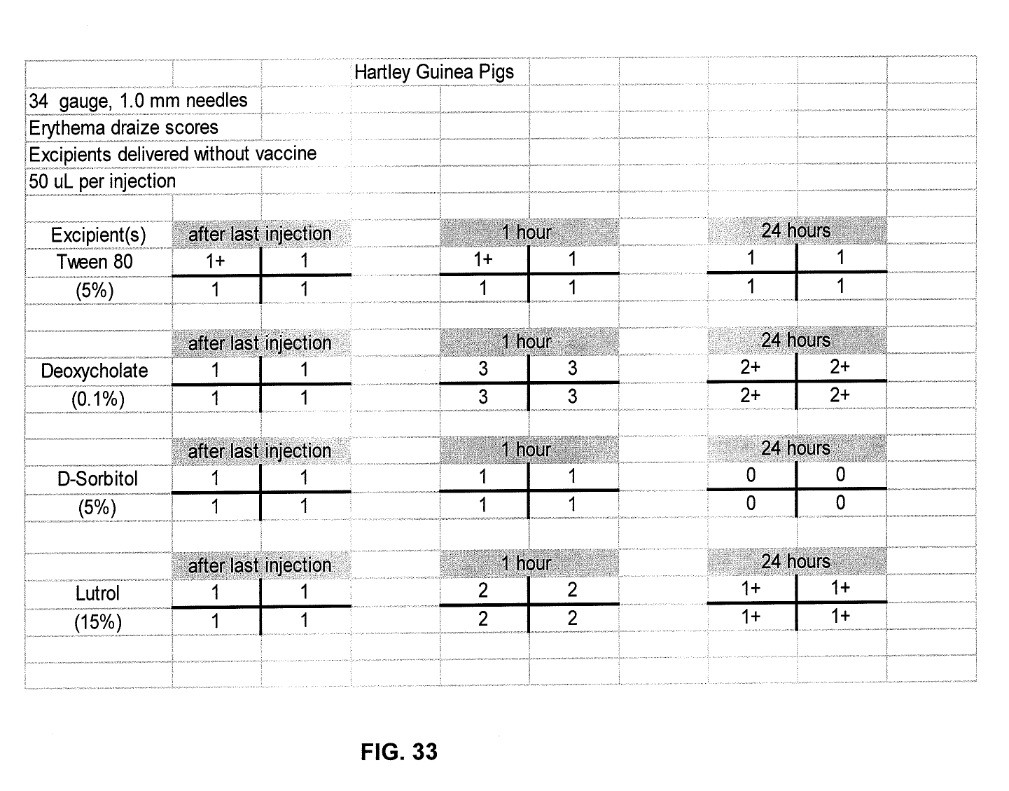

Erythema Draize scores for various excipients were determined using procedures described in Section 5.4, above. In one study, Tween™ 80 (5%), Deoxycholate (0.1%), D-sorbitol (5%) or Lutrol™ (15%) was administered (50 μl per injection) without the antigen to Hartley guinea pigs using 34 gauge, 1.0 mm needles. As shown in FIG. 33, all of the excipients were reasonably well-tolerated at the specified concentrations in guinea pigs, except for the DOC that produced skin reactions just above the acceptable draize score. Deeper administrations will be necessary for deoycholate to be used reliably at this concentration. From left to right, the reading immediately after administration, the one-hour reading and the 24-hour reading.

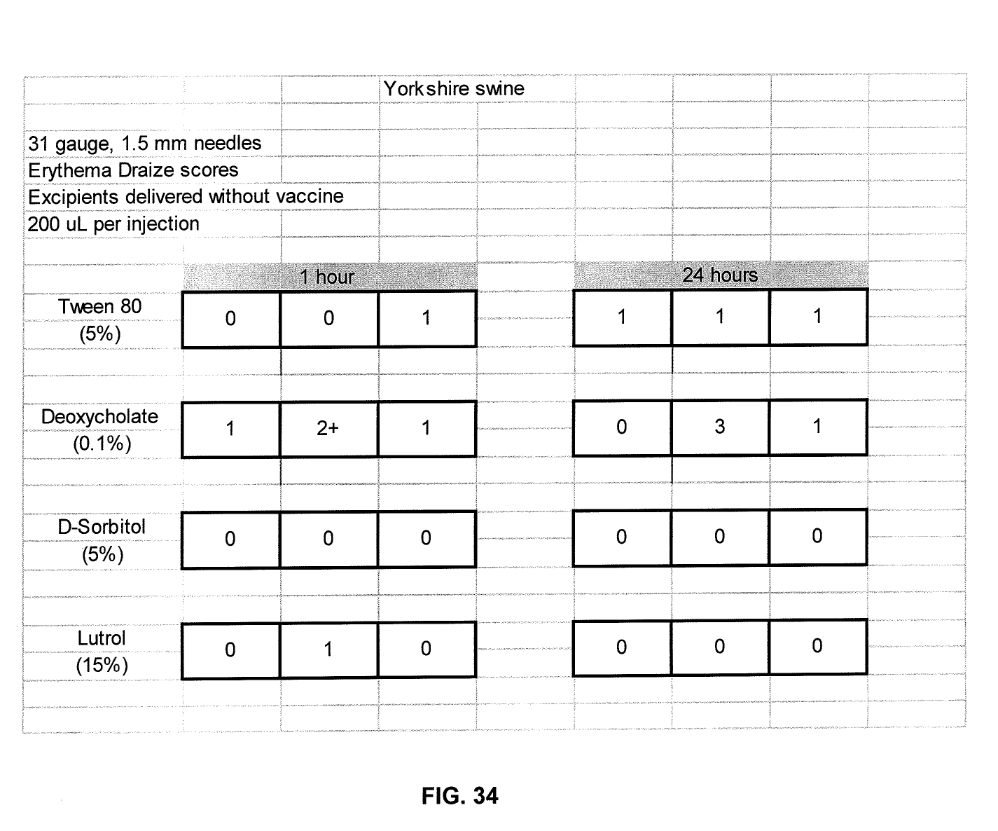

In another study, Tween™ 80 (5%), Deoxycholate (0.1%), D-sorbitol (5%) or Lutrol™ (15%) was administered (200 μl per injection) without the antigen to Yorkshire swine using 31 gauge, 1.5 mm needles. As shown in FIG. 34, all of the excipients were also reasonably well-tolerated at the specified concentrations in swine, except for the DOC that produced skin reactions just above the acceptable draize score. Deeper administrations will be necessary for deoycholate to be used reliably at this concentration. The one-hour reading (left) and the 24-hour reading (right).

While the invention has been described with respect to the particular embodiments, it will be apparent to those skilled in the art that various changes and modifications may be made without departing from the spirit and scope of the invention as recited by the appended claims.

That which is claimed:

1. A vaccine composition comprising an immunogenic agent and an excipient, wherein the excipient is a bile salt.

2. The vaccine composition of claim 1, wherein the bile salt is deoxycholate.

3. The vaccine composition of claim 2, wherein the deoxycholate is at a concentration of about 0.07 to about 0.15 percent weight per volume of the composition.

4. The vaccine composition of claim 2, wherein the deoxycholate is at a concentration of about 0.07 to about 0.60 percent weight per volume of the composition.

5. The vaccine composition of claim 1, wherein the immunogenic agent comprises at least one antigen from an influenza virus.

6. The vaccine composition of claim 5, wherein the immunogenic agent is at least one influenza virus hemagglutinin protein.

7. The vaccine composition of claim 1, wherein the immunogenic agent comprises at least one inactivated influenza virus.

8. The vaccine composition of claim 7, wherein the at least one inactivated influenza virus is selected from the group consisting of H1N1, H3N2, type B, and any combination thereof.

9. The vaccine composition of claim 7, wherein the immunogenic agent is a combination of H1N1, H3N2, and type B inactivated influenza viruses.

10. A method of eliciting an enhanced immune response from an immunogenic composition in a subject comprising delivering the vaccine composition of claim 1 into an intradermal compartment of the subject's skin.

11. The method of claim 10, wherein the vaccine composition elicits a Draize score that is equal to or less than two.
